SEQUENCE LISTING (238 sequences)

SEQ ID NO: 1 - DNA, 11 bp, Artificial sequence
  Artificially synthesized sequence
aaagttcgttt

SEQ ID NO: 2 - DNA, 9 bp, Artificial sequence
  Artificially synthesized sequence
cctgtcaac

SEQ ID NO: 3 - DNA, 11 bp, Artificial sequence
  Artificially synthesized sequence
cagaagataga

SEQ ID NO: 4 - DNA, 9 bp, Artificial sequence
  Artificially synthesized sequence
tcaccacta

SEQ ID NO: 5 - DNA, 9 bp, Artificial sequence
  Artificially synthesized sequence
tttcttgaa

SEQ ID NO: 6 - DNA, 9 bp, Artificial sequence
  Artificially synthesized sequence
aggtcttta

SEQ ID NO: 7 - DNA, 9 bp, Artificial sequence
  Artificially synthesized sequence
gttcggaca

SEQ ID NO: 8 - DNA, 9 bp, Artificial sequence
  Artificially synthesized sequence
agctctttt

SEQ ID NO: 9 - DNA, 9 bp, Artificial sequence
  Artificially synthesized sequence
aggcacatt

SEQ ID NO: 10 - DNA, 9 bp, Artificial sequence
  Artificially synthesized sequence
gtcctactt

SEQ ID NO: 11 - DNA, 11 bp, Artificial sequence
  Artificially synthesized sequence
ccttctccaac

SEQ ID NO: 12 - DNA, 11 bp, Artificial sequence
  Artificially synthesized sequence
ttcaagaaggt

SEQ ID NO: 13 - DNA, 9 bp, Artificial sequence
  Artificially synthesized sequence
tgaatgctt

SEQ ID NO: 14 - DNA, 9 bp, Artificial sequence
  Artificially synthesized sequence
gtagccaaa

SEQ ID NO: 15 - DNA, 9 bp, Artificial sequence
  Artificially synthesized sequence
caaccgtct

SEQ ID NO: 16 - DNA, 9 bp, Artificial sequence
  Artificially synthesized sequence
attcacact

SEQ ID NO: 17 - DNA, 9 bp, Artificial sequence
  Artificially synthesized sequence
tcaatcctt

SEQ ID NO: 18 - DNA, 9 bp, Artificial sequence
  Artificially synthesized sequence
tactttatc

SEQ ID NO: 19 - DNA, 10 bp, Artificial sequence
  Artificially synthesized sequence
tttatgacag

SEQ ID NO: 20 - DNA, 9 bp, Artificial sequence
  Artificially synthesized sequence
ttccaaagg

SEQ ID NO: 21 - DNA, 9 bp, Artificial sequence
  Artificially synthesized sequence
ggttgaaga

SEQ ID NO: 22 - DNA, 10 bp, Artificial sequence
  Artificially synthesized sequence
aagttccatc

SEQ ID NO: 23 - DNA, 12 bp, Artificial sequence
  Artificially synthesized sequence
taagtagacatt

SEQ ID NO: 24 - DNA, 9 bp, Artificial sequence
  Artificially synthesized sequence
tcttatcat

SEQ ID NO: 25 - DNA, 9 bp, Artificial sequence
  Artificially synthesized sequence
attttctta

SEQ ID NO: 26 - DNA, 9 bp, Artificial sequence
  Artificially synthesized sequence
tatatggag

SEQ ID NO: 27 - DNA, 10 bp, Artificial sequence
  Artificially synthesized sequence
ccacagcagt

SEQ ID NO: 28 - DNA, 9 bp, Artificial sequence
  Artificially synthesized sequence
ttgtctgtt

SEQ ID NO: 29 - DNA, 9 bp, Artificial sequence
  Artificially synthesized sequence
gtagaatgt

SEQ ID NO: 30 - DNA, 9 bp, Artificial sequence
  Artificially synthesized sequence
ctgcatgtg

SEQ ID NO: 31 - DNA, 9 bp, Artificial sequence
  Artificially synthesized sequence
ctatggctt

SEQ ID NO: 32 - DNA, 9 bp, Artificial sequence
  Artificially synthesized sequence
aagaagaag

SEQ ID NO: 33 - DNA, 9 bp, Artificial sequence
  Artificially synthesized sequence
aagtgagat

SEQ ID NO: 34 - DNA, 9 bp, Artificial sequence
  Artificially synthesized sequence
acttcacca

SEQ ID NO: 35 - DNA, 9 bp, Artificial sequence
  Artificially synthesized sequence
acaccttaa

SEQ ID NO: 36 - DNA, 10 bp, Artificial sequence
  Artificially synthesized sequence
aatgttgtct

SEQ ID NO: 37 - DNA, 9 bp, Artificial sequence
  Artificially synthesized sequence
aagttggac

SEQ ID NO: 38 - DNA, 9 bp, Artificial sequence
  Artificially synthesized sequence
tgacatcat

SEQ ID NO: 39 - DNA, 9 bp, Artificial sequence
  Artificially synthesized sequence
accgtctat

SEQ ID NO: 40 - DNA, 9 bp, Artificial sequence
  Artificially synthesized sequence
gaggacttt

SEQ ID NO: 41 - DNA, 9 bp, Artificial sequence
  Artificially synthesized sequence
aattggttg

SEQ ID NO: 42 - DNA, 9 bp, Artificial sequence
  Artificially synthesized sequence
agaatttgt

SEQ ID NO: 43 - DNA, 9 bp, Artificial sequence
  Artificially synthesized sequence
tgctatagt

SEQ ID NO: 44 - DNA, 9 bp, Artificial sequence
  Artificially synthesized sequence
caaacactt

SEQ ID NO: 45 - DNA, 11 bp, Artificial sequence
  Artificially synthesized sequence
acaaccgtcta

SEQ ID NO: 46 - DNA, 10 bp, Artificial sequence
  Artificially synthesized sequence
gagagagaga

SEQ ID NO: 47 - DNA, 9 bp, Artificial sequence
  Artificially synthesized sequence
attttgcag

SEQ ID NO: 48 - DNA, 9 bp, Artificial sequence
  Artificially synthesized sequence
cgctgattt

SEQ ID NO: 49 - DNA, 10 bp, Artificial sequence
  Artificially synthesized sequence
agacattgtc

SEQ ID NO: 50 - DNA, 9 bp, Artificial sequence
  Artificially synthesized sequence
gctacctgc

SEQ ID NO: 51 - DNA, 9 bp, Artificial sequence
  Artificially synthesized sequence
aatatcgtg

SEQ ID NO: 52 - DNA, 10 bp, Artificial sequence
  Artificially synthesized sequence
gtacattggt

SEQ ID NO: 53 - DNA, 11 bp, Artificial sequence
  Artificially synthesized sequence
tgtcagggcgt

SEQ ID NO: 54 - DNA, 9 bp, Artificial sequence
  Artificially synthesized sequence
agccatcca

SEQ ID NO: 55 - DNA, 9 bp, Artificial sequence
  Artificially synthesized sequence
gaaccaata

SEQ ID NO: 56 - DNA, 9 bp, Artificial sequence
  Artificially synthesized sequence
tacaaccct

SEQ ID NO: 57 - DNA, 9 bp, Artificial sequence
  Artificially synthesized sequence
tagaatgtc

SEQ ID NO: 58 - DNA, 11 bp, Artificial sequence
  Artificially synthesized sequence
tgccagcatga

SEQ ID NO: 59 - DNA, 9 bp, Artificial sequence
  Artificially synthesized sequence
aaggtccat

SEQ ID NO: 60 - DNA, 9 bp, Artificial sequence
  Artificially synthesized sequence
acgtccatt

SEQ ID NO: 61 - DNA, 9 bp, Artificial sequence
  Artificially synthesized sequence
acccaacta

SEQ ID NO: 62 - DNA, 11 bp, Artificial sequence
  Artificially synthesized sequence
atccaaccatg

SEQ ID NO: 63 - DNA, 10 bp, Artificial sequence
  Artificially synthesized sequence
taagagccca

SEQ ID NO: 64 - DNA, 9 bp, Artificial sequence
  Artificially synthesized sequence
tctgaggct

SEQ ID NO: 65 - DNA, 11 bp, Artificial sequence
  Artificially synthesized sequence
gtgcaacagga

SEQ ID NO: 66 - DNA, 9 bp, Artificial sequence
  Artificially synthesized sequence
gttgtagta

SEQ ID NO: 67 - DNA, 9 bp, Artificial sequence
  Artificially synthesized sequence
ccactagct

SEQ ID NO: 68 - DNA, 10 bp, Artificial sequence
  Artificially synthesized sequence
attggttgtc

SEQ ID NO: 69 - DNA, 9 bp, Artificial sequence
  Artificially synthesized sequence
gatgcacca

SEQ ID NO: 70 - DNA, 9 bp, Artificial sequence
  Artificially synthesized sequence
tgttggtag

SEQ ID NO: 71 - DNA, 9 bp, Artificial sequence
  Artificially synthesized sequence
caatcctta

SEQ ID NO: 72 - DNA, 9 bp, Artificial sequence
  Artificially synthesized sequence
gccattgtg

SEQ ID NO: 73 - DNA, 9 bp, Artificial sequence
  Artificially synthesized sequence
caacaggaa

SEQ ID NO: 74 - DNA, 9 bp, Artificial sequence
  Artificially synthesized sequence
aattaccgg

SEQ ID NO: 75 - DNA, 9 bp, Artificial sequence
  Artificially synthesized sequence
gttggtaga

SEQ ID NO: 76 - DNA, 9 bp, Artificial sequence
  Artificially synthesized sequence
gctgatttg

SEQ ID NO: 77 - DNA, 9 bp, Artificial sequence
  Artificially synthesized sequence
agatagaga

SEQ ID NO: 78 - DNA, 9 bp, Artificial sequence
  Artificially synthesized sequence
caataattt

SEQ ID NO: 79 - DNA, 9 bp, Artificial sequence
  Artificially synthesized sequence
gaaacggtg

SEQ ID NO: 80 - DNA, 9 bp, Artificial sequence
  Artificially synthesized sequence
ggcaatttt

SEQ ID NO: 81 - DNA, 9 bp, Artificial sequence
  Artificially synthesized sequence
ctatagaga

SEQ ID NO: 82 - DNA, 9 bp, Artificial sequence
  Artificially synthesized sequence
agctttctt

SEQ ID NO: 83 - DNA, 9 bp, Artificial sequence
  Artificially synthesized sequence
gcaaagaac

SEQ ID NO: 84 - DNA, 9 bp, Artificial sequence
  Artificially synthesized sequence
taatttatg

SEQ ID NO: 85 - DNA, 11 bp, Artificial sequence
  Artificially synthesized sequence
aaagttgagtc

SEQ ID NO: 86 - DNA, 9 bp, Artificial sequence
  Artificially synthesized sequence
tacaataat

SEQ ID NO: 87 - DNA, 9 bp, Artificial sequence
  Artificially synthesized sequence
gtgttagct

SEQ ID NO: 88 - DNA, 9 bp, Artificial sequence
  Artificially synthesized sequence
ccaacttcc

SEQ ID NO: 89 - DNA, 9 bp, Artificial sequence
  Artificially synthesized sequence
agagagcta

SEQ ID NO: 90 - DNA, 9 bp, Artificial sequence
  Artificially synthesized sequence
aatttgtgg

SEQ ID NO: 91 - DNA, 10 bp, Artificial sequence
  Artificially synthesized sequence
tggtgcaggt

SEQ ID NO: 92 - DNA, 9 bp, Artificial sequence
  Artificially synthesized sequence
ttgagttgg

SEQ ID NO: 93 - DNA, 9 bp, Artificial sequence
  Artificially synthesized sequence
agagtgagc

SEQ ID NO: 94 - DNA, 9 bp, Artificial sequence
  Artificially synthesized sequence
ggtaaattg

SEQ ID NO: 95 - DNA, 9 bp, Artificial sequence
  Artificially synthesized sequence
gcggtggtt

SEQ ID NO: 96 - DNA, 9 bp, Artificial sequence
  Artificially synthesized sequence
acgactctg

SEQ ID NO: 97 - DNA, 9 bp, Artificial sequence
  Artificially synthesized sequence
aaactggta

SEQ ID NO: 98 - DNA, 9 bp, Artificial sequence
  Artificially synthesized sequence
acagtgtta

SEQ ID NO: 99 - DNA, 9 bp, Artificial sequence
  Artificially synthesized sequence
tcagttggt

SEQ ID NO: 100 - DNA, 9 bp, Artificial sequence
  Artificially synthesized sequence
ttgaaggtc

SEQ ID NO: 101 - DNA, 9 bp, Artificial sequence
  Artificially synthesized sequence
acaaagtca

SEQ ID NO: 102 - DNA, 12 bp, Artificial sequence
  Artificially synthesized sequence
agtcattttctt

SEQ ID NO: 103 - DNA, 9 bp, Artificial sequence
  Artificially synthesized sequence
catgtcaaa

SEQ ID NO: 104 - DNA, 9 bp, Artificial sequence
  Artificially synthesized sequence
gactagaga

SEQ ID NO: 105 - DNA, 9 bp, Artificial sequence
  Artificially synthesized sequence
ttgaaattc

SEQ ID NO: 106 - DNA, 9 bp, Artificial sequence
  Artificially synthesized sequence
actttccat

SEQ ID NO: 107 - DNA, 11 bp, Artificial sequence
  Artificially synthesized sequence
ttgaaattacc

SEQ ID NO: 108 - DNA, 9 bp, Artificial sequence
  Artificially synthesized sequence
tttgaaatt

SEQ ID NO: 109 - DNA, 9 bp, Artificial sequence
  Artificially synthesized sequence
gtgatgtta

SEQ ID NO: 110 - DNA, 9 bp, Artificial sequence
  Artificially synthesized sequence
aggaaagta

SEQ ID NO: 111 - DNA, 9 bp, Artificial sequence
  Artificially synthesized sequence
aaattgttg

SEQ ID NO: 112 - DNA, 9 bp, Artificial sequence
  Artificially synthesized sequence
atttgtggg

SEQ ID NO: 113 - DNA, 9 bp, Artificial sequence
  Artificially synthesized sequence
tgtacattg

SEQ ID NO: 114 - DNA, 9 bp, Artificial sequence
  Artificially synthesized sequence
aatatcacc

SEQ ID NO: 115 - DNA, 11 bp, Artificial sequence
  Artificially synthesized sequence
gctgtggaaga

SEQ ID NO: 116 - DNA, 9 bp, Artificial sequence
  Artificially synthesized sequence
cttgaaatt

SEQ ID NO: 117 - DNA, 9 bp, Artificial sequence
  Artificially synthesized sequence
aactttgaa

SEQ ID NO: 118 - DNA, 9 bp, Artificial sequence
  Artificially synthesized sequence
aggtgctga

SEQ ID NO: 119 - DNA, 9 bp, Artificial sequence
  Artificially synthesized sequence
aaatggcag

SEQ ID NO: 120 - DNA, 9 bp, Artificial sequence
  Artificially synthesized sequence
gaggaaagt

SEQ ID NO: 121 - DNA, 9 bp, Artificial sequence
  Artificially synthesized sequence
agttgcaaa

SEQ ID NO: 122 - DNA, 9 bp, Artificial sequence
  Artificially synthesized sequence
aagatagag

SEQ ID NO: 123 - DNA, 10 bp, Artificial sequence
  Artificially synthesized sequence
acggcatttc

SEQ ID NO: 124 - DNA, 10 bp, Artificial sequence
  Artificially synthesized sequence
tgagttgagt

SEQ ID NO: 125 - DNA, 9 bp, Artificial sequence
  Artificially synthesized sequence
ttccaaaca

SEQ ID NO: 126 - DNA, 9 bp, Artificial sequence
  Artificially synthesized sequence
taagctctt

SEQ ID NO: 127 - DNA, 12 bp, Artificial sequence
  Artificially synthesized sequence
ttccattgttca

SEQ ID NO: 128 - DNA, 10 bp, Artificial sequence
  Artificially synthesized sequence
gctgagccac

SEQ ID NO: 129 - DNA, 9 bp, Artificial sequence
  Artificially synthesized sequence
attgaatga

SEQ ID NO: 130 - DNA, 9 bp, Artificial sequence
  Artificially synthesized sequence
agatggtgt

SEQ ID NO: 131 - DNA, 11 bp, Artificial sequence
  Artificially synthesized sequence
gtagcgactgt

SEQ ID NO: 132 - DNA, 9 bp, Artificial sequence
  Artificially synthesized sequence
ttttcatgt

SEQ ID NO: 133 - DNA, 11 bp, Artificial sequence
  Artificially synthesized sequence
ttcttgaactt

SEQ ID NO: 134 - DNA, 10 bp, Artificial sequence
  Artificially synthesized sequence
cttggactga

SEQ ID NO: 135 - DNA, 9 bp, Artificial sequence
  Artificially synthesized sequence
cttgaactg

SEQ ID NO: 136 - DNA, 9 bp, Artificial sequence
  Artificially synthesized sequence
ccagccatt

SEQ ID NO: 137 - DNA, 9 bp, Artificial sequence
  Artificially synthesized sequence
aatctgtca

SEQ ID NO: 138 - DNA, 9 bp, Artificial sequence
  Artificially synthesized sequence
agtgacagt

SEQ ID NO: 139 - DNA, 9 bp, Artificial sequence
  Artificially synthesized sequence
tcatccaat

SEQ ID NO: 140 - DNA, 9 bp, Artificial sequence
  Artificially synthesized sequence
ctttgaaat

SEQ ID NO: 141 - DNA, 9 bp, Artificial sequence
  Artificially synthesized sequence
atcatccaa

SEQ ID NO: 142 - DNA, 9 bp, Artificial sequence
  Artificially synthesized sequence
ggctctttc

SEQ ID NO: 143 - DNA, 9 bp, Artificial sequence
  Artificially synthesized sequence
atgtggtgg

SEQ ID NO: 144 - DNA, 9 bp, Artificial sequence
  Artificially synthesized sequence
ctcgatcgt

SEQ ID NO: 145 - DNA, 11 bp, Artificial sequence
  Artificially synthesized sequence
ttgttcactgt

SEQ ID NO: 146 - DNA, 9 bp, Artificial sequence
  Artificially synthesized sequence
gggctgttc

SEQ ID NO: 147 - DNA, 9 bp, Artificial sequence
  Artificially synthesized sequence
aggactcag

SEQ ID NO: 148 - DNA, 9 bp, Artificial sequence
  Artificially synthesized sequence
ctaagaaga

SEQ ID NO: 149 - DNA, 9 bp, Artificial sequence
  Artificially synthesized sequence
tctccctct

SEQ ID NO: 150 - DNA, 9 bp, Artificial sequence
  Artificially synthesized sequence
ttgagttga

SEQ ID NO: 151 - DNA, 9 bp, Artificial sequence
  Artificially synthesized sequence
cttggtaat

SEQ ID NO: 152 - DNA, 9 bp, Artificial sequence
  Artificially synthesized sequence
tcagtaagt

SEQ ID NO: 153 - DNA, 9 bp, Artificial sequence
  Artificially synthesized sequence
agagttgtt

SEQ ID NO: 154 - DNA, 9 bp, Artificial sequence
  Artificially synthesized sequence
agataacca

SEQ ID NO: 155 - DNA, 9 bp, Artificial sequence
  Artificially synthesized sequence
attgttcat

SEQ ID NO: 156 - DNA, 9 bp, Artificial sequence
  Artificially synthesized sequence
tctgacttc

SEQ ID NO: 157 - DNA, 9 bp, Artificial sequence
  Artificially synthesized sequence
caacttccg

SEQ ID NO: 158 - DNA, 9 bp, Artificial sequence
  Artificially synthesized sequence
tttccattg

SEQ ID NO: 159 - DNA, 9 bp, Artificial sequence
  Artificially synthesized sequence
agttgttca

SEQ ID NO: 160 - DNA, 9 bp, Artificial sequence
  Artificially synthesized sequence
gtcagggcg

SEQ ID NO: 161 - DNA, 9 bp, Artificial sequence
  Artificially synthesized sequence
aaattgaat

SEQ ID NO: 162 - DNA, 9 bp, Artificial sequence
  Artificially synthesized sequence
tgatctttc

SEQ ID NO: 163 - DNA, 9 bp, Artificial sequence
  Artificially synthesized sequence
tctgtagct

SEQ ID NO: 164 - DNA, 9 bp, Artificial sequence
  Artificially synthesized sequence
ttggtaatt

SEQ ID NO: 165 - DNA, 9 bp, Artificial sequence
  Artificially synthesized sequence
ttttgcagt

SEQ ID NO: 166 - DNA, 9 bp, Artificial sequence
  Artificially synthesized sequence
ttggaaagt

SEQ ID NO: 167 - DNA, 9 bp, Artificial sequence
  Artificially synthesized sequence
ggcattttc

SEQ ID NO: 168 - DNA, 9 bp, Artificial sequence
  Artificially synthesized sequence
ttcttgctg

SEQ ID NO: 169 - DNA, 10 bp, Artificial sequence
  Artificially synthesized sequence
caaaactgag

SEQ ID NO: 170 - DNA, 9 bp, Artificial sequence
  Artificially synthesized sequence
gccatgcta

SEQ ID NO: 171 - DNA, 9 bp, Artificial sequence
  Artificially synthesized sequence
aggtgtgtc

SEQ ID NO: 172 - DNA, 9 bp, Artificial sequence
  Artificially synthesized sequence
ttatcattg

SEQ ID NO: 173 - DNA, 9 bp, Artificial sequence
  Artificially synthesized sequence
tacaaagta

SEQ ID NO: 174 - DNA, 9 bp, Artificial sequence
  Artificially synthesized sequence
acaatgaat

SEQ ID NO: 175 - DNA, 9 bp, Artificial sequence
  Artificially synthesized sequence
tgtgatgtt

SEQ ID NO: 176 - DNA, 9 bp, Artificial sequence
  Artificially synthesized sequence
aaaatgact

SEQ ID NO: 177 - DNA, 9 bp, Artificial sequence
  Artificially synthesized sequence
tgtcgtctc

SEQ ID NO: 178 - DNA, 9 bp, Artificial sequence
  Artificially synthesized sequence
ttataagct

SEQ ID NO: 179 - DNA, 9 bp, Artificial sequence
  Artificially synthesized sequence
tggtaattg

SEQ ID NO: 180 - DNA, 9 bp, Artificial sequence
  Artificially synthesized sequence
gttataagc

SEQ ID NO: 181 - DNA, 9 bp, Artificial sequence
  Artificially synthesized sequence
ttgaactgt

SEQ ID NO: 182 - DNA, 9 bp, Artificial sequence
  Artificially synthesized sequence
ccctcttta

SEQ ID NO: 183 - DNA, 9 bp, Artificial sequence
  Artificially synthesized sequence
actaagaag

SEQ ID NO: 184 - DNA, 9 bp, Artificial sequence
  Artificially synthesized sequence
aaactgagg

SEQ ID NO: 185 - DNA, 9 bp, Artificial sequence
  Artificially synthesized sequence
ggaaaatga

SEQ ID NO: 186 - DNA, 9 bp, Artificial sequence
  Artificially synthesized sequence
gtctgactt

SEQ ID NO: 187 - DNA, 9 bp, Artificial sequence
  Artificially synthesized sequence
gttggaaag

SEQ ID NO: 188 - DNA, 9 bp, Artificial sequence
  Artificially synthesized sequence
tgttgacat

SEQ ID NO: 189 - DNA, 9 bp, Artificial sequence
  Artificially synthesized sequence
aatgatgga

SEQ ID NO: 190 - DNA, 9 bp, Artificial sequence
  Artificially synthesized sequence
actggtaga

SEQ ID NO: 191 - DNA, 9 bp, Artificial sequence
  Artificially synthesized sequence
gttgtctgt

SEQ ID NO: 192 - DNA, 9 bp, Artificial sequence
  Artificially synthesized sequence
tgttgtctg

SEQ ID NO: 193 - DNA, 9 bp, Artificial sequence
  Artificially synthesized sequence
tgaggcttg

SEQ ID NO: 194 - DNA, 9 bp, Artificial sequence
  Artificially synthesized sequence
tggattgaa

SEQ ID NO: 195 - DNA, 9 bp, Artificial sequence
  Artificially synthesized sequence
tcttttgag

SEQ ID NO: 196 - DNA, 9 bp, Artificial sequence
  Artificially synthesized sequence
ccttggggc

SEQ ID NO: 197 - DNA, 9 bp, Artificial sequence
  Artificially synthesized sequence
gtgatcttt

SEQ ID NO: 198 - DNA, 9 bp, Artificial sequence
  Artificially synthesized sequence
tttggcaat

SEQ ID NO: 199 - DNA, 21 bp, Artificial sequence
  Artificially synthesized sequence
ttccacagctttcttgaactg

SEQ ID NO: 200 - DNA, 21 bp, Artificial sequence
  Artificially synthesized sequence
gaggaaagtatcgccttctag

SEQ ID NO: 201 - DNA, 22 bp, Artificial sequence
  Artificially synthesized sequence
ctcttctgcctgctgcactttg

SEQ ID NO: 202 - DNA, 22 bp, Artificial sequence
  Artificially synthesized sequence
atgggctacaggcttgtcactc

SEQ ID NO: 203 - DNA, 22 bp, Artificial sequence
  Artificially synthesized sequence
ccacagaccttccaggagaatg

SEQ ID NO: 204 - DNA, 23 bp, Artificial sequence
  Artificially synthesized sequence
gtgcagttcagtgatcgtacagg

SEQ ID NO: 205 - DNA, 24 bp, Artificial sequence
  Artificially synthesized sequence
tgtatgtgactgcccaagatgaag

SEQ ID NO: 206 - DNA, 22 bp, Artificial sequence
  Artificially synthesized sequence
agaggaggttggtctcactacc

SEQ ID NO: 207 - DNA, 22 bp, Artificial sequence
  Artificially synthesized sequence
agacagccactcacctcttcag

SEQ ID NO: 208 - DNA, 22 bp, Artificial sequence
  Artificially synthesized sequence
ttctgccagtgcctctttgctg

SEQ ID NO: 209 - DNA, 20 bp, Artificial sequence
  Artificially synthesized sequence
tctatggcccagaccctcac

SEQ ID NO: 210 - DNA, 20 bp, Artificial sequence
  Artificially synthesized sequence
gacggcagagaggaggttga

SEQ ID NO: 211 - DNA, 23 bp, Artificial sequence
  Artificially synthesized sequence
gtgtgccgtctttcattacacag

SEQ ID NO: 212 - DNA, 24 bp, Artificial sequence
  Artificially synthesized sequence
cagaccctcacactcagatcatct

SEQ ID NO: 213 - DNA, 20 bp, Artificial sequence
  Artificially synthesized sequence
atcagtacctcacggctgct

SEQ ID NO: 214 - DNA, 20 bp, Artificial sequence
  Artificially synthesized sequence
tgggtatctcaggcatctcc

SEQ ID NO: 215 - DNA, 24 bp, Artificial sequence
  Artificially synthesized sequence
gagaggagacttcacagaggatac

SEQ ID NO: 216 - DNA, 20 bp, Artificial sequence
  Artificially synthesized sequence
gtactccagaagaccagagg

SEQ ID NO: 217 - DNA, 20 bp, Artificial sequence
  Artificially synthesized sequence
ggtcggtgtgaacggatttg

SEQ ID NO: 218 - DNA, 22 bp, Artificial sequence
  Artificially synthesized sequence
ggagtcatactggaacatgtag

SEQ ID NO: 219 - DNA, 21 bp, Artificial sequence
  Artificially synthesized sequence
gtatgacaacagcctcaagat

SEQ ID NO: 220 - DNA, 20 bp, Artificial sequence
  Artificially synthesized sequence
gtccttccacgataccaaag

SEQ ID NO: 221 - DNA, 20 bp, Artificial sequence
  Artificially synthesized sequence
accgtagctggtgtctctat

SEQ ID NO: 222 - DNA, 22 bp, Artificial sequence
  Artificially synthesized sequence
gtgccaaccaccatagaatttg

SEQ ID NO: 223 - DNA, 40 bp, Artificial sequence
  Artificially synthesized sequence
cagagaaggagctcggtaccatggctgatgcacaatcgtt

SEQ ID NO: 224 - DNA, 45 bp, Artificial sequence
  Artificially synthesized sequence
tatctagatccggtggatccttataagactgtatgcggtgtgtac

SEQ ID NO: 225 - DNA, 20 bp, Artificial sequence
  Artificially synthesized sequence
tgctggagcaggacggaatc

SEQ ID NO: 226 - DNA, 21 bp, Artificial sequence
  Artificially synthesized sequence
tctacaaatgtggtatggctg

SEQ ID NO: 227 - DNA, 31 bp, Artificial sequence
  Artificially synthesized sequence
aatgtctgacgtaaagcctctaaaatgtctg

SEQ ID NO: 228 - DNA, 15 bp, Artificial sequence
  Artificially synthesized sequence
cctctaaaatgtctg

SEQ ID NO: 229 - DNA, 15 bp, Artificial sequence
  Artificially synthesized sequence
cagacattttagagg

SEQ ID NO: 230 - DNA, 15 bp, Artificial Sequence
  Artificially synthesized sequence
tgttactttccttta

SEQ ID NO: 231 - DNA, 15 bp, Artificial Sequence
  Artificially synthesized sequence
cacactttcctcgtg

SEQ ID NO: 232 - RNA, 21 nt, Artificial Sequence
  Artificially synthesized sequence
gaucuuccgcuaugaaaggag

SEQ ID NO: 233 - RNA, 21 nt, Artificial Sequence
  Artificially synthesized sequence
gaucucccgcuauaacaaaag

SEQ ID NO: 234 - DNA, 15 bp, Artificial Sequence
  Artificially synthesized sequence
cataagaaagctaca

SEQ ID NO: 235 - RNA, 21 nt, Artificial Sequence
  Artificially synthesized sequence
gucaaguucuuucgacaccuu

SEQ ID NO: 236 - RNA, 21 nt, Artificial Sequence
  Artificially synthesized sequence
gucaaguccgguugacaccuu

SEQ ID NO: 237 - RNA, 21 nt, Artificial Sequence
  Artificially synthesized sequence
gaggaaaguaucgccuucuag

SEQ ID NO: 238 - RNA, 21 nt, Artificial Sequence
  Artificially synthesized sequence
uuccacagcuuucuugaacug

(19) United States
(12) Patent Application Publication
(10) Pub. No.: US 2023/0201244 A1
(43) Pub. Date: Jun. 29, 2023

(54) COMPOSITIONS AND METHODS FOR PREVENTING AND/OR TREATING MICROBIAL INFECTIONS

(71) Applicant: University of Louisville Research Foundation, Inc., Louisville, KY (US)

(72) Inventor: Huang-Ge Zhang, Louisville (US)

(73) Assignee: University of Louisville Research Foundation, Inc., Louisville (US)

(21) Appl. No.: 17/927,111

(22) PCT Filed: May 24, 2021
(86) PCT No.: PCT/US2021/033913

§ 371 Date: Nov. 22, 2022

Related U.S. Application Data
(60) Provisional application No. 63/093,528, filed on Oct. 19, 2020.
(60) Provisional application No. 63/030,001, filed on May 26, 2020.
(60) Provisional application No. 63/029,088, filed on May 22, 2020.

(51) Int. Cl. A61K 31/713; A61K 45/06; A61K 36/9066; A61K 36/8962; A61K 36/752; A61K 9/51; A61P 31/14

(52) U.S. Cl. CPC A61K 31/713; A61K 45/06; A61K 36/9066; A61K 36/8962; A61K 36/752; A61K 9/51; A61P 31/14; B82Y 5/00

(57) ABSTRACT

Provided are methods for inhibiting immune responses against microbial antigens. In some embodiments, the methods include administering to the subject a composition containing a plurality of plant-derived exosome-like particles to inhibit immune esponses against microbial antigens. Also provided are methods for inhibiting development of septic shock in subjects, for inhibiting development of cytokine storm in subjects, and for inhibiting SARS-CoV-2-induced cytopathogenic effect. Also provided are compositions that include an exosome-derived nanoparticle comprising a first lipid bilayer and a second lipid bilayer coating the exosome-like nanoparticle and/or fused with the first lipid bilayer, wherein the second lipid bilayer comprises a targeting molecule, and methods for using the compositions to treat diseases, disorders, and conditions and/or to target a therapeutic agent to a cell, tissue, and/or organ of interest.

### CROSS REFERENCE TO RELATED APPLICATION

This application claims the benefit of U.S. Provisional Patent Application Ser. Nos. 63/029,088, filed May 22, 2020; 63/030,011, filed May 26, 2020; and 63/093,528, filed Oct. 19, 2020. The disclosure of each of these U.S. Provisional Patent Applications is incorporated herein by reference in its entirety.

### GOVERNMENT INTEREST

This invention was made with government support under grant number AT008617 awarded by National Institutes of Health. The government has certain rights in the invention.

### REFERENCE TO SEQUENCE LISTING SUBMITTED ELECTRONICALLY

The content of the electronically submitted sequence listing in ASCII text file (Name: 1577_90_PCT_ST25.txt; Size: 49 kilobytes; and Date of Creation: May 24, 2021) filed with the instant application is incorporated herein by reference in its entirety.

### TECHNICAL FIELD

The presently disclosed subject matter relates generally to methods for inhibiting undesirable levels of and/or consequences of anti-microbial immune responses including but not limited to antiviral immune responses, such as but not limited to septic shock and/or cytokine storm, in subject in need thereof. In some embodiments, the methods comprise administering to a subject in need thereof a composition comprising garlic-derived exosomes (G-Exo) and/or turmeric-derived exosomes (T-Exo), and/or lemon-derived exosomes (L-Exo), wherein the G-Exo and/or the T-Exo and/or the L-Exo are present in the composition individually and/or collectively in amounts sufficient to inhibit the undesirable level of and/or consequences of the anti-microbial immune responses in the subject.

### BACKGROUND

Sepsis is a potentially life-threatening complication of an infection including COVID-19. Recent estimates place the annual deaths due to sepsis worldwide in excess of 11 million. Each year, at least 1.7 million adults in America develop sepsis. Nearly 270,000 Americans die as a result of sepsis. 1 in 3 patients who die in a hospital have sepsis.

Sepsis is mostly the consequence of systemic bacterial infections leading to exacerbated activation of immune cells by bacterial products, resulting in enhanced release of inflammatory mediators. Lipopolysaccharide (LPS), the major component of the outer membrane of Gram-negative bacteria, is a critical factor in the pathogenesis of sepsis. Exposure to LPS and other immune response-inducing compounds can result in the development of cytokine storm, which is a severe immune reaction in which the release of cytokines is inadequately regulated, thereby causing various negative consequences including but not limited to high fever, inflammation, fatigue, and nausea. Cytokine storm can also lead to organ failure, and thus is a particularly dangerous result of microbial infections.

As a result, antagonists capable of blocking cytokine storm would be of great value in limiting the dangerous consequences of infections. Disclosed herein is the discovery that garlic exosome-like nanoparticles (G-Exo), turmeric root exosome-like nanoparticles (T-Exo), and lemon-derived exosomes (L-Exo) can inhibit LPS-induced septic shock via non-invasive intranasal administration, suggesting that G-Exo and/or T-Exo and/or L-Exo are excellent candidates for treating and/or preventing septic shock, cytokine storm, and other consequences of microbial infection in mammals.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter relates in some embodiments to methods for inhibiting undesirable levels of and/or consequences of immune responses against microbial antigens, optionally immune responses against viral antigens, in subject in need thereof. In some embodiments, the methods comprise administering to a subject in need thereof a composition comprising garlic-derived exosomes (G-Exo) and/or turmeric-derived exosomes and/or lemon-derived exosomes (L-Exo), wherein the G-Exo and/or the T-Exo and/or the L-Exo are present in the composition individually and/or collectively in amounts sufficient to inhibit the immune response against the microbial, optionally virus, antigen in the subject.

The presently disclosed subject matter also relates in some embodiments to methods for inhibiting development of septic shock in subject in need thereof. In some embodiments, the methods comprise administering to a subject in need thereof a composition comprising garlic-derived exosomes (G-Exo) and/or turmeric-derived exosomes (T-Exo) and/or lemon-derived exosomes (L-Exo) in an amount and via a route sufficient to inhibit development of septic shock in the subject.

The presently disclosed subject matter also relates in some embodiments to methods for inhibiting development of cytokine storm in subjects in need thereof. In some embodiments, the methods comprise administering to a subject in need thereof a composition comprising garlic-derived exosomes (G-Exo) and/or turmeric-derived exosomes (T-Exo) and/or lemon-derived exosomes (L-Exo) in an amount and via a route sufficient to inhibit development of cytokine storm in the subject.

In some embodiments of the presently disclosed methods, the composition is administered to the subject by inhalation and/or insufflation, optionally where in inhalation and/or insufflation occurs through the nasal cavity.

In some embodiments of the presently disclosed methods, the microbial antigen is a virus antigen, optionally a coronavirus antigen, further optionally an antigen derived from a SARS-CoV-2 virus.

In some embodiments, the presently disclosed subject matter also relates to methods for treating viral infections, optionally coronavirus infections, further optionally SARS-CoV-2 infections, the method comprising administering to a subject in need thereof an effective amount of a composition and/or a pharmaceutical composition as disclosed herein.

In some embodiments, the presently disclosed subject matter also relates to methods for inhibiting a SARS-CoV-2 induced cytopathogenic effect (CPE), the method comprising administering to a subject in need thereof an effective amount of a composition and/or a pharmaceutical composition as disclosed herein, wherein the composition and/or the pharmaceutical composition comprises an miR396a species, an miR-rL1-28 species, or any combination thereof. In some embodiments, the composition and/or the pharmaceutical composition comprises a ginger exosome-like nanoparticle (GELN), a garlic exosome-like particle (G-Exo), a turmeric exosome-like particle (T-Exo), a lemon (L-Exo) exosome-like particle, or any combination thereof comprising an miRNA aly-miR396a-5p.

In some embodiments, the presently disclosed subject matter also relates to methods for targeting therapeutics to cells, tissues, and/or organs of interest. In some embodiments, the methods comprise administering to a subject in need thereof an effective amount of a composition and/or a pharmaceutical composition as disclosed herein, wherein the composition and/or the pharmaceutical composition comprises a targeting molecule that targets a therapeutic molecule to the cell, tissue, or organ of interest.

In some embodiments of the presently disclosed methods, the subject is a human.

In some embodiments, the methods of the presently disclosed subject matter further comprise administering to the subject one or more antimicrobial and/or antiviral treatments and/or one or more immunosuppressive treatments.

In some embodiments, the presently disclosed subject matter also relates to compositions. In some embodiments, the presently disclosed compositions comprise n exosome-derived nanoparticle comprising a first lipid bilayer; and a second lipid bilayer coating the exosome-like nanoparticle and/or fused with the first lipid bilayer, wherein the second lipid bilayer comprises a targeting molecule that targets the composition to a cell, tissue, or organ of interest. In some embodiments, the exosome-derived nanoparticle encapsulates a therapeutic agent. In some embodiments, the second lipid bilayer is derived from a virus, optionally a coronavirus, further optionally a SARS-CoV-2 virus. In some embodiments, the exosome-derived nanoparticle is derived from an edible plant, optionally a fruit, vegetable, or other plant. In some embodiments, the exosome-derived nanoparticle is derived from a grape, a grapefruit, a tomato, broccoli, ginger (e.g., is a GELN), garlic (e.g., is a G-Exo), turmeric (e.g., is a T-Exo), and/or lemon (e.g., is a L-Exo). In some embodiments, the therapeutic agent is selected from a phytochemical agent, a chemotherapeutic agent, and an antimicrobial agent, optionally an antiviral agent. In some embodiments, the therapeutic agent is a phytochemical agent, optionally a phytochemical agent selected from curcumin, resveratrol, baicalein, equol, fisetin, and quercetin. In some embodiments, the therapeutic agent is a chemotherapeutic agent, optionally a chemotherapeutic agent selected from the group consisting of retinoic acid, 5-fluorouracil, vincristine, actinomycin D, adriamycin, cisplatin, docetaxel, doxorubicin, and taxol. In some embodiments, the therapeutic agent comprises a nucleic acid molecule selected from an siRNA, a microRNA, and a mammalian expression vector. In some embodiments, the antiviral agent is a nucleotide or nucleoside analogue, and/or is an anti-retroviral agent.

In some embodiments, the presently disclosed subject matter also relates to pharmaceutical compositions comprising a composition as disclosed herein and a pharmaceutically-acceptable vehicle, carrier, or excipient.

In some embodiments, the presently disclosed subject matter also relates to any and all compositions, devices, systems, apparatuses, uses, and/or methods shown and/or described expressly or by implication in the information provided herewith, including but not limited to features that may be apparent and/or understood by those of skill in the art.

Thus, it is an object of the presently disclosed subject matter to provide methods for inhibiting undesirable levels of and/or consequences of immune responses against microbial antigens in subjects in need thereof.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1D show the results of experiments demonstrating that garlic exosomes-like nanoparticles (G-Exo) inhibited septic shock-induced by LPS in a mouse model. Lipopolysaccharide (18.5 mg/kg, Sigma-Aldrich) was injected i.p. into C57BL/6j mice, one hour after LPS injection, mice (n=5) were intranasal given G-Exo or T-Exo (2 μl/drop for both sides of nose, for 5 times). An equal amount of PBS was used as diluents in the control. At day 4 after the treatment, mice were euthanized and examined. FIG. 1A is a series of plots of cellular targeting of PKH26-labeled T-Exo or G-Exo determined by flow cytometry analyses of collagenase-digested lung tissue. The panels represent cells positive for CD11b, CD11c, GR1, and CD3, respectively, from left to right. The percentages of cells positive for uptake of PKH26-labeled exosomes is shown in each plot. FIG. 1B is a series of bar graphs summarizing the percentage of CD11b⁺, CD11c⁺, GR1⁺, and CD3⁺ cells (respectively from left to right in each panel) that were positive for T-Exo uptake (top panel) or G-Exo update (bottom panel) in frozen sections stained with F4/80. A representative image from each group (n=5) is shown. FIG. 1C is a graph of mouse mortality over a period of 3 days for negative control (PBS-treated), T-Exo-treated, and G-Exo-treated mice. FIG. 1D is a series of micrographs of formalin fixed lung sections stained with H&E. Representative images from negative control (PBS-treated), T-Exo-treated, and G-Exo-treated mice (n=5 per group) is shown.

FIG. 2 is a schematic diagram of a strategy for producing a pseudo-SARS-CoV-2 particle by encapsulating a plant-derived exosome with a lipid bilayer derived from SARS-CoV-2 and using the same to treat SARS-CoV-2-induced lung inflammation. It is noted that while a broccoli-derived exosome-like nanoparticle is depicted and SARS-CoV-2 is depicted as providing the membrane-derived vesicle with which to coat the exosome-like nanoparticle, other exosome-like nanoparticles, including from other plants or other species including but not limited to humans (e.g., a human cell that expresses on its surface a desired targeting molecule) can also be employed to generate membrane-derived vesicles with which to coat other exosome-like nanoparticles. Thus, it is understood that the depicts of the viral membranes and the exosome-like nanoparticles are exemplary only and not to be construed as limiting the presently disclosed subject matter.

FIGS. 3A-3M. Lung epithelial cells release exosome containing Nsp12 of SARS-CoV-2 that enhances the inflammatory response in lung. FIG. 3A. Schematic representation of the treatment schedule for the effect of lung epithelial cell-derived exosomes containing SARS-CoV-2 proteins on lung immune cells. FIG. 3B. SARS-CoV-2 protein expression plasmids transfected into lung epithelial A549 cells. Representative blots of viral proteins in exosomes and cells as well as exosomal marker CD63 by western blot using StrepTactin-HRP conjugate and antibody to CD63. FIG. 3C. Intensity of GFP fuse-expressed with spike (S) protein in exosomes (Exo) and cells using BioTek's SYNERGY™ Microplate Reader. FIG. 3D. Representative western blot of exosomes from Vero E2 cells transfected with Nsp12 and Nsp13 plasmids. FIG. 3E. Cytokines in the medium assessed by ELISA. FIG. 3F. Schematic representation of intratracheal injection (left panel) and a mouse undergoing laryngoscopy to expose the vocal cords (right panel). FIGS. 3G and 3H. Exosome from mouse lung LLC1 cells transfected with SARS-CoV-2 plasmids administrated to C57BL/6 mice (5×10⁸/kg, body weight, n=5) by intratracheal injection. After 24 h, frequency of F4/80⁺ cells (FIG. 3G), Gr-1⁺ cells. FIG. 3H) and PKH26-labeled exosomes in the lung from C57BL/6 mice assessed using flow cytometry. Numbers in boxes indicate percent of exosome/PKH26⁺ cells. FIG. 3I. Quantification of percentage of exosomes/PKH26⁺ in F4/80⁺ cells and Gr-1⁺ cells. FIG. 3J. Assessment of cytokines in the lungs using ELISA. FIG. 3K. Cytokines in the F4/80⁺ cells assessed by flow cytometry (top panel). Quantification of data from flow cytometry (bottom panel). FIG. 3L. Representative hematoxylin and eosin (H&E)-stained sections of formalin-fixed, paraffin-embedded lungs (400× magnification, scale bar: 200 μm) from

C57BL/6 mice. FIG. 3M. A549 cells co-transfected with the plasmids of pAcGFP1-C-Nsp12-Flag and pLVX-Nsp13-Strep. 72 h after transfection, Nsp12/13 complex pull-down by Strep-Tactin XT magnetic beads and dot immunoblot analysis with anti-Flag antibody. Data are representative of three independent experiments (error bars, SD). *p<0.05 and **p<0.01 (two-tailed t-test).

FIGS. 4A-4J. GELN miRNA targets to the RNA of the SARS-CoV-2. FIG. 4A. Flow diagram depicting the steps taken in identifying unique GELN miRNAs targeting to the RNA of the SARS-CoV-2. FIG. 4B. Venn diagram of miRNAs detected in the ginger tissue and GELNs using miRNA sequencing. FIG. 4C. Heatmap showing miRNAs from ginger tissue, GELNs, human and mouse brain (n=3 per group). FIG. 4D. Waterfall plot showing the differs in the relative abundance of miRNAs between GELNs and ginger tissue normalized by human miRNAs. FIG. 4E. Distribution of RNA biotypes differs. Boxes represent median and interquartile ranges. FIG. 4F. Schematic diagram and distribution of the putative binding sites of GELN miRNAs in the full length SARS-CoV-2 genome. UTR, untranslated regions. The miRNAs of human and mouse that have the same mapping seed sequences as GELNs are indicated by the second arrow down on the right of FIG. 4F and were excluded for further experiments. FIGS. 4G and 4H. Predicted consequential pairing of target region of spike gene (FIG. 4G, top, including SEQ ID NOs: 230 and 231), Nsp12 gene (FIG. 4H, top; SEQ ID NO: 234) and GELN rlcv-miR-rL1-28-3p (FIG. 4G, bottom; SEQ ID NO: 232), aly-miR396a-5p (FIG. 4H, bottom; SEQ ID NO: 235), respectively. The miRNAs seed matches in the target RNAs are mutated at the positions as indicated (SEQ ID NO: 233 for rlcv-miR-rL1-28-3p and SEQ ID NO: 236 for aly-miR396a-5p). FIG. 4I. A549 cells transfected with CoV-2 S inserted into pcDNA3-GFP and GELN rlcv-miR-rL1-28-3p, mutant RNA. Visualization with confocal fluorescence microscopy. FIG. 4J. A549 cells transfected with Nsp12 inserted into pLVX-EF1alpha-2xStrep-IRES and GELN aly-miR396a-5p, mutant RNA. Visualization with StrepTactin-HRP conjugate by immunoblot. Data are representative of three independent experiments. Error bars are ±SD. *p<0.05 and **p<0.01 (two-tailed t-test).

FIGS. 5A-5G. Aly-miR396a-5p reduces NF-κB activated by Nsp12/13 through phosphorylation of IKKβ. FIG. 5A. Western blot analysis showing the phosphorylation (p) of IKKβ, IκBα, NF-κB (p65). JNK as well as total NF-κB (p65) in macrophages of the lung in C57BL/6 mice (n=5) inoculated by intratracheal administration with exosomes (5×10⁸/kg, body weight) from LLC1 cells transfected with Nsp12 and/or Nsp13 as well as aly-miR396a-5p. Arrows mark the positions of p54 and p46 subunits of p-JNK. GAPDH served as a loading control. FIG. 5B. Pretreat with p-JNK inhibitor (SP600125, 5 mg/kg/d, body weight) and p-IxBa inhibitor (Bay 11-7821, 10 mg/kg/d, body weight) (n=5) by intraperitoneal injection 3 days following intratracheal administration of exosomes. Western blot analysis showing the p-IKBα, p-JNK and p-p65 in lung macrophages. FIG. 5C. Western blot analysis of cleaved (c) caspase-3, c-caspase-7 and p-PARP in the lung of mice. FIG. 5D. Analysis of apoptosis by TUNEL staining in lung tissues. The TUNEL assay revealed apoptotic-positive cells in lung marked by GFP staining. The blue DAPI stain marks intact DNA. Magnification, ×400 (up panel). Quantification of TUNEL positive cells (bottom panel). The data were collected by counting positive cells from 3 lung sections of specimens and shown as mean±SD vs Vehicle, **p<0.01. NS: not significant. FIG. 5E. Analysis of apoptosis by flow cytometry using Annexin V-FITC staining in EpCAM⁺ cells of lung from mice. Numbers in boxes indicate a representative percent of EpCAM⁺ apoptotic cells (top panel). The adjunct histograms display the univariate plots that correspond to the EpCAM in the bivariate plot. Quantification of percentage of EpCAM⁺Annexin V⁺7-AAD⁻ cells (bottom panel). Data are representative of three independent experiments (error bars, SD). *p<0.05 and **p<0.01 (two-tailed t-test). FIG. 5F. Analysis of apoptosis by flow cytometry in lung epithelial A549 cells presented to Nsp12/13, Bay 11-7821 (top panel), or culture supernatant from U937 macrophages treated with A549-derived Nsp12/13 exosomes with or without Bay 11-7821 (middle panel), anti-TNFα, anti-IL-1β and anti-IL-6 antibodies (10 ng/ml, bottom panel), respectively. Numbers in boxes indicate a representative percent of Annexin V⁺7-AAD⁻ apoptotic cells. Quantification of percentage of Annexin V⁺7-AAD⁻ cells (right panel). Data are representative of three independent experiments (error bars, SD). vs Nsp12/13 group, *p<0.05 and **p<0.01 (two-tailed t-test). FIG. 5G. Proposed model for the crosstalk between GELN miR396a-5p regulate cytokine expression mediated by SARS-CoV-2 Nsp12 in a manner dependent of NF-kB signaling.

FIGS. 6A-6J. GELN aly-miR396a-5p suppresses the expression of cytokines mediated by Nsp12/Nsp13 synergy. FIG. 6A. GELN-derived nanovectors (GNVs, 10 mg) administrated to C57BL/6 mice (n=5) by intratracheal injection. Representative flow cytometry plots showing GELN-derived nanovector (GNV) stained with PKH26 in F4/80⁺ cells (left) and EpCAM⁺ cells (right) of lungs 12 h after intratracheal injection. FIG. 6B. Western blot analysis expression of Nsp12-Strep and spike protein in lungs with StrepTactin-HRP conjugate and anti-S antibody 48 h after administration of viral plasmid CoV-2-Nsp12-2xStrep and pcDNA3-CoV-2-S, as well as GNVs packing aly-miR396a-5p and rlcv-miR-rL1-28 or appropriate mutant RNA, respectively, by intratracheal injection. Numbers below the western blots represent densitometry values normalized to the loading control. GAPDH served as a loading control. FIG. 6C. ELISA analysis showing the level of TNFα, IL-1β and IL-6 in human macrophage U937 cells transfected with Nsp12 and/or Nsp13 as well as aly-miR396a-5p. FIG. 6D. ELISA analysis showing the level of TNFα, IL-1β and IL-6 in the lungs inoculated with Nsp12 and/or Nsp13 as well as aly-miR396a-5p through intratracheal administration. Nsp12/13 vs Nsp12 or Nsp13, *p<0.05, **p<0.01; Nsp12/13+miR396a-5p vs Nsp12/13, #p<0.05, ##p<0.01. FIG. 6E. Analysis of cytokine levels in lungs from C57BL/6 mice with indicated treatment in the figures through intratracheal administration using a mouse cytokine array (n=3). FIG. 6F. Quantification of relative intensity of the selective up-regulation and down-regulation of cytokines shown in the cytokine array. FIG. 6G. Cytokines in the F4/80⁺ cells assessed by flow cytometry (top panel). Quantification of data from flow cytometry (bottom panel). FIG. 6H. Cytokines in the F4/80⁺ cells assessed by qPCR. FIG. 6I. Representative H&E-stained sections of lungs (400× magnification, scare bar: 200 μm). FIG. 6J. The unwinding activity of SARS-Nsp13 and SARS-Nsp12 with or without aly-miR396a-5p is presented as the time-course changing of the dsDNA unwound fraction. The initial dsDNA concentration is 250 nM and the protein concentration is 20 nM. Nsp12/13 vs Nsp13; Nsp12/13 vs Nsp12/13+miR396a-5p. Data are representative of three independent experiments. Error bars are ±SD. *p<0.05 and **p<0.01 (two-tailed t-test).

FIGS. 7A-7E. GELN miRNAs inhibit cytopathic effects (CPE) of Vero E2 infected with SARS-CoV-2. FIG. 7A. Schematic representation of the treatment schedule for the effect of GELN miRNAs on the CPE of Vero E2 cells infected with the SARS-CoV-2. FIG. 7B. qPCR analysis of spike (top panel) and Nsp12 (bottom panel) gene product expression in Vero E2 cells after 72 h infection with SARS-CoV-2 at a MOI of 0.003. FIG. 7C. Western blot analysis of spike protein in transfected Vero E2 cells. Numbers below the western blot represent densitometry values normalized to the loading control. FIG. 7D. 2×10⁴ Vero cells in 96-well plates exposed to 60 pfu of SARS-CoV-2 and GELN miRNAs as well as control indicated in the graph. A representative CPE estimated at 72 h post infection. Scale bars, 100 μm (left panel). Semi-quantitative analysis of CPE at four levels; <25%, 25%, 50%, >50% from three independent experiments (right panel). FIG. 7E. Proposed model of SARS-CoV-2 activation of cytokines in lung macrophage mediated by exosome cargo of viral protein from infected epithelial cells and GELN miRNA extinguish the activation of cytokines in lung by directly targeting the viral gene of SARS-CoV-2. Data are representative of three independent experiments (error bars, SD). *p<0.05 and **p<0.01 (two-tailed t-test).

FIGS. 8A-8C. Lung epithelial cells release exosome containing the proteins of SARS-CoV-2. FIG. 8A. A549 transfected with pcDNA3-CoV-2-S-GFP. Visualization with confocal fluorescence microscopy. Scale bars, 20 μm. FIG. 8B. Analysis of dot blot with anti-GFP antibody. FIG. 8C. U937 cells treated with exosomes from A549 cells and cytokine analysis in the medium with ELISA. Data are representative of three independent experiments. Error bars are ±SD.

FIGS. 9A-9F. Distribution of lung epithelial cell-released exosomes following intratracheal administration in mice. FIG. 9A. A representative fluorescence image of brain, lung, heart, liver, kidney, small intestine and large intestine from C57BL/6 mice receiving a single intratracheal administration of 10 mg DiR dye-labelled LCC1-derived exosome at 0 h, 1 h and 2 h (left panel); Image of serum after intratracheal administration (right panel). FIGS. 9B and 9C. Representative immunofluorescence in the lung from C57BL/6 mice receiving a single intratracheal administration of 10 mg PKH26-labelled LCC1-derived exosome at 24 h. Visualization of F4/80⁺, Gr-1⁺ (FIG. 9B) and CD-11b⁺ (FIG. 9C) cells by confocal microscopy. Arrows identify exosome/PKH26 taken up by F4/80⁺ or Gr-1⁺ cells; Scale bars, 20 μm. FIG. 9D. ELISA analysis of TNFα, IL-1β, and IL-6 in serum from C57BL/6 mice three days after inoculation of LCC1-derived exosome containing Nsp12, Nsp13, or Nsp12/13 through intratracheal administration. FIG. 9E. ELISA analysis of TNFα, IL-1β, and IL-6 in serum from C57BL/6 mice three days after inoculation of LLC1-derived exosome containing Nsp12, Nsp13, or Nsp12/13 through intratracheal administration. FIG. 9F. Exosomes from primary lung epithelial cells transfected with Nsp12 and Nsp13 plasmids and administrated to mice via intratracheal injection. ELISA analysis of TNFα, IL-1β, and IL-6 in lung. Data are representative of three independent experiments. Error bars are ±SD. *p<0.05 and **p<0.01 (two-tailed t-test).

FIGS. 10A-10E. Nsp12/13 activate cytokines mediated by NFκB pathway. FIG. 10A. Western blot analysis of the phosphorylation of MAPK (p38), ERK 1/2 (p44/42) and PI3K in lung macrophages of C57BL/6 mice (n=5) after intratracheal inoculation with exosomes from LCC1 cells transfected with Nsp12 and/or Nsp13 as well as aly-miR396a-5p. Data are representative of three independent experiments. FIG. 10B. Pretreatment with p-IκBα inhibitor (Bay 11-7821, 10 mg/kg/d, body weight; n=5) by intraperitoneal injection 3 days following intratracheal administration of exosomes. ELISA analysis of TNFα and IL-6 in lung macrophages. FIG. 10C. Representative immunofluorescence in lung from C57BL/6 mice receiving Bay 11-7821 (10 mg/kg/d, body weight; n=5) by intraperitoneal injection 3 days following a single intratracheal administration of 10 mg of exosomes with Nsp12/13 per day for three consecutive days. Visualization of TUNEL-GFP⁺ and EpCAM⁺ cells by confocal microscopy. Arrows identify TUNEL⁺EpCAM⁺ cells; Scale bars, 20 μm. FIG. 10D. The exosome^{Nsp12/13} and exosome^{Nsp12/13+miR396a-5p} from LLC1 cells intratracheally injected into mice. The apoptotic bodies (ABs) were isolated from lung epithelial cells and quantified with FACS using forward scatter (F SC) and Annexin V-FITC staining. FIG. 10E. ELISA analysis of cytokines in the lung of mice intratracheally injected with ABs at 1×10⁸. Data are representative of three independent experiments. Error bars are ±SD. *p<0.05 and **p<0.01 (two-tailed t-test).

FIGS. 11A-11E. Purification and characterization of ginger-derived nanovesicles (GNVs). FIG. 11A. Sucrose-banded particles from ginger juice. The nanoparticles were isolated from ginger juice using a sucrose gradient (8, 30, 45, and 60% sucrose in 20 mM Tri-C1, pH 7.2). Particles from the band between 8% and 30% sucrose were used for preparation of nanoparticles. FIG. 11B. Size distribution of GNVs using a NanoSight NS300 (Westborough, Mass.) with a flow speed at 0.03 mL per min. FIG. 11C. Quantification of GNV yield (n=3) by weight of lipid from GELN. Data are representative of three independent experiments. Error bars are ±SD. FIG. 11D. A representative electron microscopy image of ginger ELNs. Scale bars, 200 nm. (FIG. 11E) A representative fluorescence image of lung (left panel) and small intestine (right panel) from C57BL/6 mice receiving a single intratracheal administration of 10 mg DiR dye-labelled GNVs at 0 hours, 1 hour, 12 hours, 24 hours, and 72 hours; Image of serum after intratracheal administration (right panel). n=5 per group. Data are representative of three independent experiments. Error bars are ±SD.

FIGS. 12A-12F. GNVs reduce the induction of cytokines activated by LPS in lung. FIG. 12A. Representative immunofluorescence in lung from C57BL/6 mice receiving a single intratracheal administration of 10 mg PKH26-labelled GNVs at 24 h. Visualization of F4/80⁺ and EpCAM⁺ cells by confocal microscopy. Arrows identify exosome/PKH26 taken up by F4/80⁺ or EpCAM⁺ cells; Scale bars, 20 μm. FIG. 12B. ELISA analysis of cytokines in lung from C57BL/6 mice receiving a single intratracheal administration of 1×10⁸ GNVs, grapefruit-derived nanovesicles (GFNVs), gold nanoparticles (NP) and 5 μg LPS at 12 h. FIG. 12C. GNVs generated with additional PA, PC, and PE. FACS analysis of GNVs/PKH26 taken up by A549 cells (top panel). Quantification of percentage of exosome/PKH26⁺ in A549 cells (bottom panel). FIG. 12D. ELISA analysis of cytokines in lung treated with LPS (1 mg/kg) via intravenous administration and vesicles for therapeutic delivery by Gold nanoparticles (NP), GNVs and grapefruit nanovesicles (GFNVs). FIG. 12E. Serum aspartate transaminase (AST) and alanine transaminase (ALT) levels of C57BL/6 mice with various concentration of GNVs intratracheal administration. FIG. 12F. Evaluation of A549 cell proliferation and cytotoxicity of GNVs with various concentration indicated in the graph using a luminescence ATP monitoring system. n=5 per group. ; *p<0.05 and **p<0.01 (two-tailed t-test). NS: not significant. Data are representative of three independent experiments. Error bars are ±SD.

FIGS. 13A-13D. GNVs efficiently deliver miRNA to lung through intratracheal injection. FIG. 13A. 10 μg of aly-miR396a-5p packed with 200 μmol GNVs using ultrasonication. The capacity of aly-miR396a-5p GELNs and GNVs using qPCR. FIG. 13B. qPCR analysis of aly-miR396a-5p in A549 cells transfected with aly-miR396a-5p GNV compared to RNAiMAX and PEI. FIG. 13C. 10 μg of aly-miR396a-5p packed into GNVs and gold NPs following intratracheal administration of C57BL/6 mice. After 48 h, qPCR analysis of aly-miR396a-5p distribution in various parts of the lung. FIG. 13D. qPCR analysis expression of Nsp12 and spike (S) protein in lung after administration of viral plasmid CoV-2-Nsp12-2xStrep and pcDNA3-CoV-2-S, as well as GNVs packing aly-miR396a-5p and rlcv-miR-rL1-28 or appropriate mutant RNA, respectively by intratracheal injection. *p<0.05 and **p<0.01 (two-tailed t-test). Data are representative of three independent experiments. Error bars are ±SD.

FIGS. 14A and 14B. Nsp12/13 reduce growth factors and CXCL family assessed by protein array. Quantification of relative intensity of the selected cytokines involving cell growth factor (FIG. 14A) and chemokine (C-X-C motif) ligand (CXCL) (FIG. 14B) shown in a cytokine array in FIG. 5E. *p<0.05 and **p<0.01 (two-tailed t-test).

### DETAILED DESCRIPTION

### I. General Considerations

Lung inflammation is a hallmark of COVID-19 and is the consequence of intercellular communications between severe acute respiratory syndrome coronavirus 2 (SARS-CoV-2) infected cells and immune cells. Unfortunately, the molecular details of SARS-CoV-2-immune cell intercommunication precludes a comprehensive evaluation of molecule candidates for host-directed therapies. Here it is shown that naïve mice develop inflamed lung tissue after being administered exosomes released from the lung epithelial cells exposed to SARS-CoV-2 Nsp12 and Nsp13 (exosomes^{Nsp12Nsp13}). Additionally, it is shown that Nsp13 has synergistic effects with Nsp12 on the induction of exosome mediated lung inflammation and apoptosis of lung epithelial cells via activation of lung macrophages. Mechanistically, it is shown that exosomes^{Nsp12Nsp13} is taken up by lung macrophages, leading to activation of NF-κB and the subsequent induction of an array of inflammatory cytokines. Induction of TNFα, IL-6 and IL-1β in the context of metabolites released from exosomes^{Nsp12Nsp13} activated lung macrophages also contributes to inducing apoptosis in lung epithelial cells. Induction of exosomes^{Nsp12Nsp13} mediated lung inflammation was abolished with ginger exosome-like nanoparticle (GELN) miRNA aly-miR396a-5p. The role of GELNs in inhibition of SARS-CoV-2 induced cytopathogenic effect (CPE) in Vero E6 cells was further demonstrated via GELN aly-miR396a-5p and rlcv-miR-rL1-28-3p mediated inhibition of expression of SARS-CoV-2 Nsp12 and spike genes, respectively. Together, these results revealed exosomes^{Nsp12Nsp13} as important contributors to the development of lung inflammation, and GELNs among others as potential therapeutic agents to treat COVID-19.

The severe cases of COVID-19 cause a cytokine storm resulting high mortality. Hyper-production of the cytokines ultimately results in tissue damage including apoptosis and necrosis, leading to injury of alveolar epithelial cells and vascular endothelial cells, as well as to lung infiltration sustained by continuously infiltrated immune cells. However, delivering viral specific therapeutic agents is challenging. The mammalian virus is known to be highly host specific and co-evolving with their hosts. SARS-CoV-2 viral factors encoded by genetic material could evolve from the host, therefore, using therapeutic agents such as miRNA therapy targets to the viral mRNA but not the host mRNA, which induce side-effects.

Anti-viral therapeutic agents derived from a kingdom that are not co-evolved with the mammalian kingdom can provide a more potent anti-viral effect with less potential to induce side effects as a result of the anti-viral therapy. Plants do not belong to the mammalian kingdom. Recently, exosome-like nanoparticles (ELN) from the tissue of edible plants have been described (Mu et al., 2014; Xiao et al., 2018). Therefore, as disclosed herein, whether therapeutic factors present in ELNs, such as but not limited to ginger ELN (GELN) miRNAs, which are unique and would not compete with host endogenous miRNA to bind SARS-CoV-2 mRNA, were investigated. In achieving this goal, there would be no interfering with endogenous miRNA regulated pathways while achieving the therapeutic effects against the expression of SARS-CoV-2 specific genes. Moreover, since multiple species of miRNAs can be encapsulated in a single ELN and each miRNA can potentially bind to multiple sites of the viral genome. Thus, the production of infectious virus is expected to be inhibited via blocking a number of pathways that are critical for generating infectious virus.

Exosomes released from virally infected cells contribute to the cytokine storm³⁻⁶. Whether exosomes released from SARS-CoV-2 infected lung epithelial cells play a role in induction of inflammation cytokines which further trigger tissue damage is not known.

Studies in mice and humans have suggested that activation of the nuclear factor-κB (NF-κB) pathway contributes to viral factors inducing the lung cytokine storm. The details of whether SARS-CoV-2 derived factors can induce lung inflammation are unknown. The SARS-CoV-2 Nsp12 RNA-dependent RNA polymerase (RdRP) and Nsp13 helicase are non-structural proteins. RdRP is an enzyme that catalyzes the synthesis of the SARS RNA strand complementarily to the SARS-CoV-2 RNA template and is thus essential to the replication of SARS-CoV-2 RNA. Like most other RNA viruses, SARS-CoV-2 RdRPs are considered to be highly conserved to maintain viral functions and for this reason are targeted in antiviral drug development as well as diagnostic tests.

The presently disclosed results support the hypothesis that SARS-CoV-2 Nsp12 induces lung inflammation mediated by exosome released from lung epithelial cells that can be inhibited by GELN derived miRNA. Nsp13 has synergistic effects with Nsp12 on the lung inflammation. These findings may shed light on therapy development for COVID-19 patients and open a new avenue for studying mechanisms underlying plant kingdom crosstalk with the mammalian kingdom via plant exosome-like nanoparticles and for developing anti-viral therapeutic strategies.

### II. Definitions

Headings are included herein for reference and to aid in locating certain sections. These headings are not intended to limit the scope of the concepts described therein under, and these concepts may have applicability in other sections throughout the entire specification. While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

In describing and claiming the presently disclosed subject matter, the following terminology will be used in accordance with the definitions set forth below.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims.

The term “about”, as used herein to refer to a measurable value such as an amount of weight, time, etc., is meant to encompass in some embodiments variations of ±20%, in some embodiments ±10%, in some embodiments ±5%, in some embodiments ±1%, in some embodiments ±0.1%, in some embodiments ±0.5%, and in some embodiments ±0.01% from the specified amount, as such variations are appropriate to perform the disclosed methods.

As used herein, the term “and/or” when used in the context of a list of entities, refers to the entities being present singly or in any possible combination or subcombination. Thus, for example, the phrase “A, B, C, and/or D” includes A, B, C, and D individually, but also includes any and all combinations and subcombinations of A, B, C, and D.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease, disorder, and/or condition which may not be responsive to the primary treatment for the injury, disease, disorder, and/or condition being treated.

As use herein, the terms “administration of” and or “administering” a compound should be understood to mean providing a compound of the presently disclosed subject matter or a prodrug of a compound of the presently disclosed subject matter to a subject in need of treatment.

As used herein, an “agonist” is a composition of matter which, when administered to a mammal such as a human, enhances or extends a biological activity attributable to the level or presence of a target compound or molecule of interest in the mammal.

A disease, disorder, and/or condition is “alleviated” if the severity of a symptom of the disease, condition, or disorder, or the frequency with which such a symptom is experienced by a subject, or both, are reduced.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a method of the presently disclosed subject matter can “consist essentially of” one or more enumerated steps as set forth herein, which means that the one or more enumerated steps produce most or substantially all of the intended result to be produced by the claimed method. It is noted, however, that additional steps can be encompassed within the scope of such a method, provided that the additional steps do not substantially contribute to the result for which the method is intended.

With respect to the terms “comprising”, “consisting essentially of”, and “consisting of”, where one of these three terms is used herein, the presently disclosed and claimed subject matter can include the use of either of the other two terms. Similarly, it is also understood that in some embodiments the methods of the presently disclosed subject matter comprise the steps that are disclosed herein, in some embodiments the methods of the presently disclosed subject matter consist essentially of the steps that are disclosed, and in some embodiments the methods of the presently disclosed subject matter consist of the steps that are disclosed herein.

As used herein, the term “subject” refers to any organism for which diagnosis and/or treatment would be desirable. Thus, the term “subject” is in some embodiments a human subject, although it is to be understood that the principles of the presently disclosed subject matter indicate that the presently disclosed subject matter is effective with respect to other species, including mammals, which are intended to be included in the term “subject”. Moreover, a mammal is understood to include any mammalian species for which diagnosis, treatment, and/or prophylaxis is desirable, particularly agricultural and domestic mammalian species. As used herein, a “subject in need thereof” is a patient, animal, mammal, or human, who will benefit from the compositions and methods of the presently disclosed subject matter.

Thus, in some embodiments the methods of the presently disclosed subject matter are particularly useful for warm-blooded vertebrates. Thus, the presently disclosed subject matter concerns mammals and birds. More particularly contemplated is the isolation, manipulation, and use of stem cells from mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also contemplated is the isolation, manipulation, and use of stem cells from livestock, including but not limited to domesticated swine (pigs and hogs), ruminants, horses, poultry, and the like.

As used herein, the phrase “substantially” refers to a condition wherein in some embodiments no more than 50%, in some embodiments no more than 40%, in some embodiments no more than 30%, in some embodiments no more than 25%, in some embodiments no more than 20%, in some embodiments no more than 15%, in some embodiments no more than 10%, in some embodiments no more than 9%, in some embodiments no more than 8%, in some embodiments no more than 7%, in some embodiments no more than 6%, in some embodiments no more than 5%, in some embodiments no more than 4%, in some embodiments no more than 3%, in some embodiments no more than 2%, in some embodiments no more than 1%, and in some embodiments no more than 0% of the components of a collection of entities does not have a given characteristic.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

The term “aqueous solution” as used herein can include other ingredients commonly used, such as sodium bicarbonate described herein, and further includes any acid or base solution used to adjust the pH of the aqueous solution while solubilizing a peptide.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

As used herein, the term “biologically active fragments” or “bioactive fragment” of the peptides encompasses natural or synthetic portions of a longer peptide or protein that are capable of specific binding to their natural ligand or of performing the desired function of the protein, for example, a fragment of a protein of larger peptide which still contains the epitope of interest and is immunogenic.

The term “biological sample”, as used herein, refers to samples obtained from a subject, including, but not limited to, skin, hair, tissue, blood, plasma, cells, sweat, and urine.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

As used herein “injecting, or applying, or administering” includes administration of a compound of the presently disclosed subject matter by any number of routes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal routes.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

The term “per application” as used herein refers to administration of a drug or compound to a subject.

The term “pharmaceutical composition” shall mean a composition comprising at least one active ingredient, whereby the composition is amenable to investigation for a specified, efficacious outcome in a mammal (for example, without limitation, a human).Those of ordinary skill in the art will understand and appreciate the techniques appropriate for determining whether an active ingredient has a desired efficacious outcome based upon the needs of the artisan.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application.

As used herein, the term “physiologically acceptable” ester or salt means an ester or salt form of the active ingredient which is compatible with any other ingredients of the pharmaceutical composition, which is not deleterious to the subject to which the composition is to be administered.

As used herein, “pharmaceutical compositions” include formulations for human and veterinary use.

“Plurality” means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

The term “prevent”, as used herein, means to stop something from happening, or taking advance measures against something possible or probable from happening. In the context of medicine, “prevention” generally refers to action taken to decrease the chance of getting a disease or condition.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The term to “treat”, as used herein, means reducing the frequency with which symptoms are experienced by a patient or subject or administering an agent or compound to reduce the frequency with which symptoms are experienced.

A “prophylactic” treatment is a treatment administered to a subject who does not exhibit signs of a disease or exhibits only early signs of the disease for the purpose of decreasing the risk of developing pathology associated with the disease.

### III. Compositions of the Presently Disclosed Subject Matter and Therapeutic Methods for Using the Same

In some embodiments, the presently disclosed subject matter relates to exosomal compositions. In some embodiments, the composition comprises, consists essentially of, or consists of an exosome-derived nanoparticle comprising a first lipid bilayer and a second lipid bilayer coating the exosome-like nanoparticle and/or fused with the first lipid bilayer, wherein the second lipid bilayer comprises a targeting molecule that targets the composition to a cell, tissue, or organ of interest.

In some embodiments, the exosomal compositions that are produced in accordance with the presently disclosed subject matter make use of exosomes and exosome-derived nanoparticles that are first isolated from a cell before the exosomes are then used to encapsulate a therapeutic agent of interest. Methods for isolating exosomes are known, and include those described in U.S. Pat. No. 10,799,457, which is incorporated herein by reference in its entirety. Additional methods are disclosed in PCT International Patent Application Publication No. WO 2013/070324 and in U.S. Patent Application Publication Nos. 2012/0315324, 2014/0308212, 2017/0035700, and 2018/0362724, each of which is also incorporated herein by reference in its entirety. It is noted that any cell can be employed to generate the exosomes and exosome-derived nanoparticles of the presently disclosed subject matter. In some embodiments, the exosome-derived nanoparticle is derived from an edible plant, optionally a fruit, vegetable, or other plant. In some embodiments, the exosome-derived nanoparticle is derived from a grape, a grapefruit, a tomato, broccoli, ginger (GELN), garlic (e.g., is a G-Exo), turmeric (e.g., is a T-Exo), and/or lemon (L-Exo).

In some embodiments, the exosome-derived nanoparticle encapsulates a therapeutic agent. More specifically, the presently-disclosed subject matter relates to nanoparticle compositions that include one or more therapeutic agents encapsulated by exosome-derived nanoparticles and that are useful in the diagnosis and treatment of disease. In some embodiments, a nanoparticle composition is provided where the therapeutic agent is selected from a phytochemical agent, a stat3 inhibitor, a chemotherapeutic agent, and an antimicrobial agent, optionally an antiviral agent. Exemplary phytochemical agents include, but are not limited to curcumin, resveratrol, baicalein, equol, fisetin, and quercetin. Exemplary Stat3 inhibitors include JSI-124 (CAS Number 2222-07-3; European Community (EC) Number 218-736-8; IUPAC Name (8S,9R,10R,13R,14S,16R,17R)-17-[(E,2R)-2,6-dihydroxy-6-methyl-3-oxohept-4-en-2-yl]-2,16-dihydroxy-4,4,9,13,14-pentamethyl-8,10,12,15,16,17-hexahydro-7H-cyclopenta[a]phenanthrene-3,11-dione; also called cucurbitacin I, Elatercin B, and NSC 521777).

In some embodiments, the therapeutic agent is a chemotherapeutic agent. Exemplary chemotherapeutic agents include, but are not limited to retinoic acid, 5-fluorouracil, vincristine, actinomycin D, adriamycin, cisplatin, docetaxel, doxorubicin, and taxol.

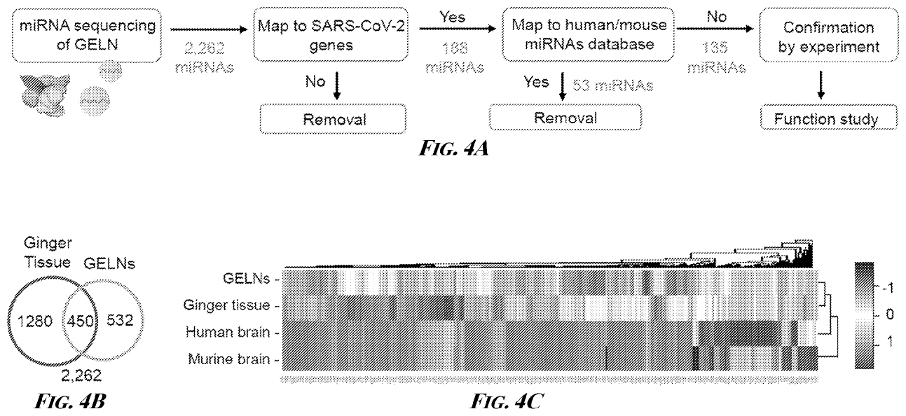

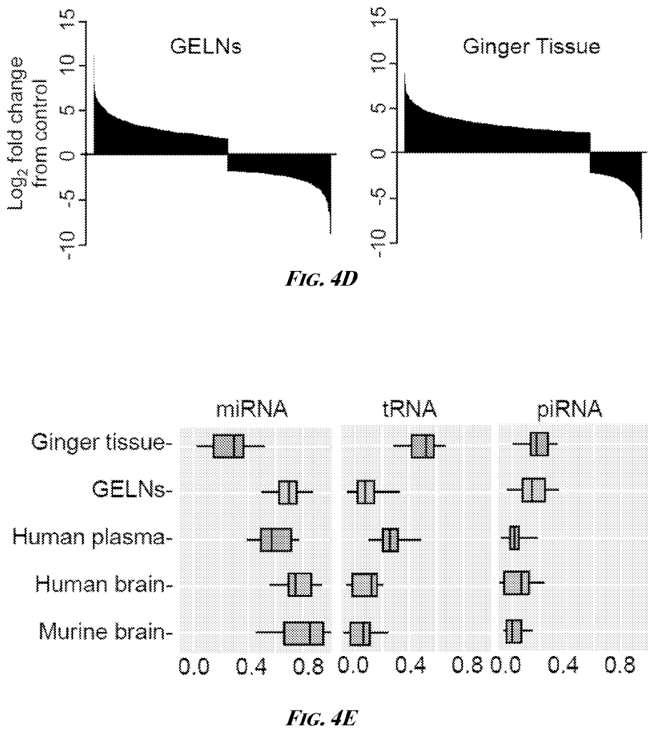

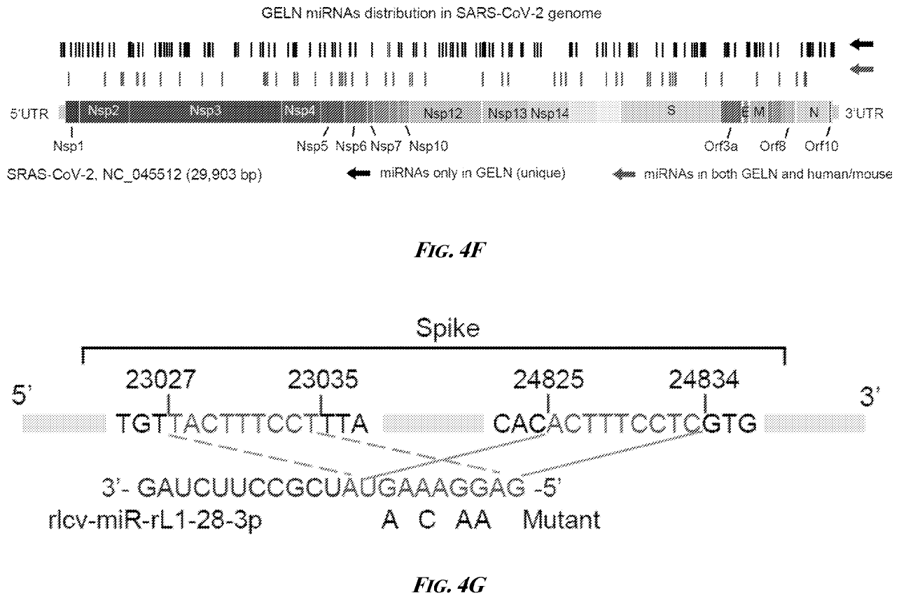

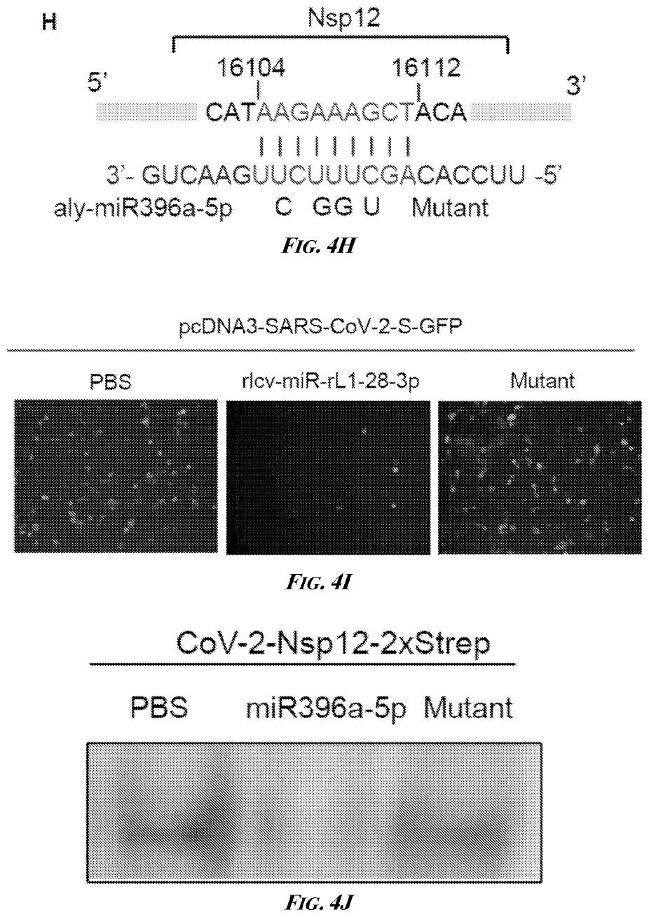

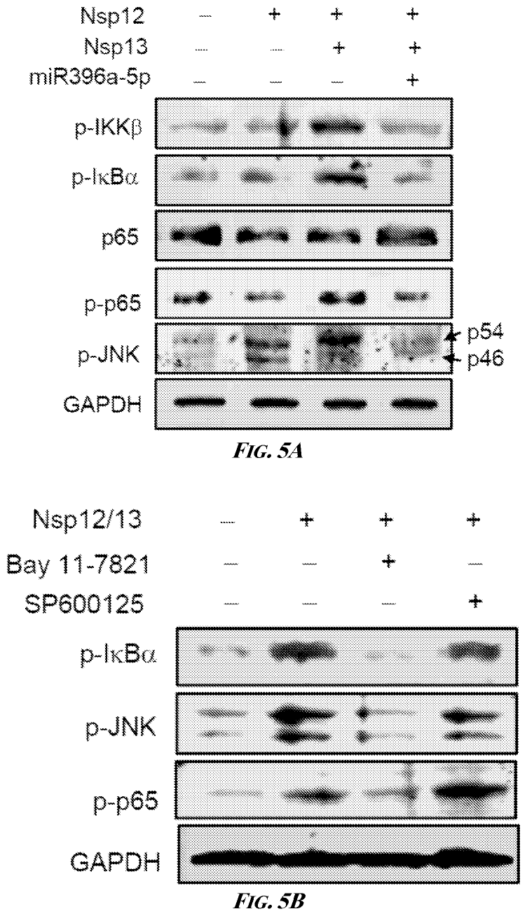

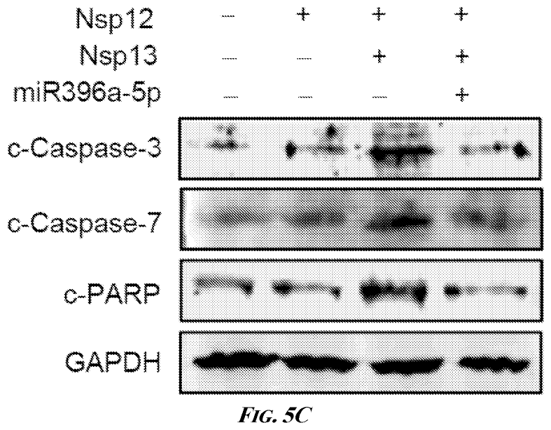

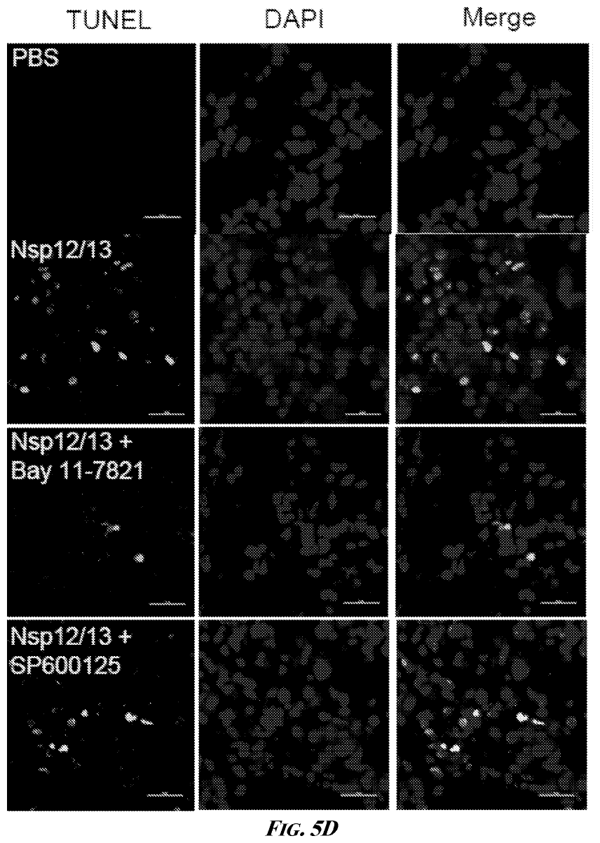

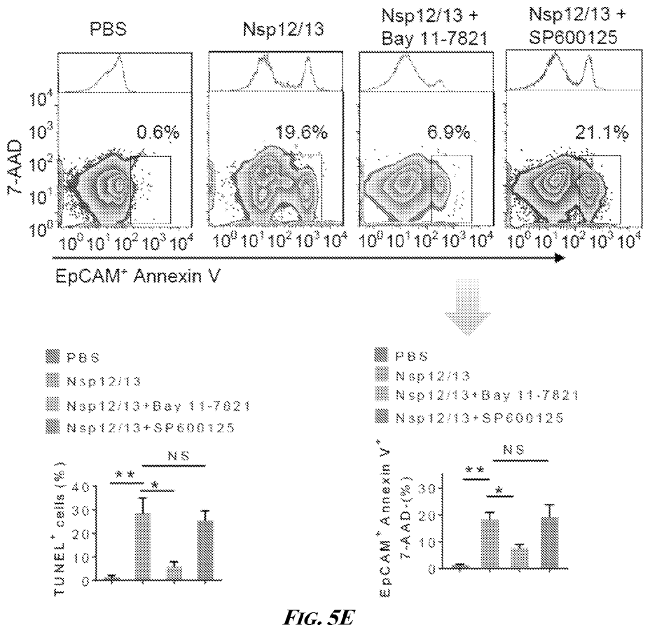

In some embodiments, the therapeutic agent comprises a nucleic acid molecule selected from an siRNA, a microRNA (miRNA), and a mammalian expression vector. In some embodiments, the nucleic acid molecule is an miRNA that targets a microbial (e.g., viral) transcription product. By way of example and not limitation, in some embodiments an miRNA can be employed that binds to and thus inhibits expression of a SARS-CoV-2 gene product, which in some embodiments can be a SARS-CoV-2 RNA that encodes the spike (S) protein or the Nsp12 polymerase. In some embodiments, the miRNA is selected from the group consisting of rlcv-miR-rL1-28-3p (GAGGAAAGUAUCGCCUUCUAG; SEQ ID NO: 237; see FIG. 4G) and aly-miR396a-5p (UUCCACAGCUUUCUUGAACUG; SEQ ID NO: 238; see FIG. 4H).

In some embodiments, the nucleic acid molecules that are encapsulated or otherwise incorporated into a microvesicle composition of the presently-disclosed subject matter are included in the microvesicles are part of an expression vector. The term “expression vector” is used interchangeably herein with the terms “expression cassette” and “expression control sequence,” and is used to refer to a nucleic acid molecule capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The coding region usually encodes a polypeptide of interest but can also encode a functional RNA of interest, for example antisense RNA or a non-translated RNA, in the sense or antisense direction. The expression vector comprising the nucleotide sequence of interest can be chimeric, meaning that at least one of its components is heterologous with respect to at least one of its other components. The expression vector can also be one that is naturally occurring but has been obtained in a recombinant form useful for heterologous expression. In some embodiments, the expression vector is a mammalian expression vector that is capable of directing expression of a particular nucleic acid sequence of interest in a mammalian cell.

In some embodiments, the therapeutic agent is an antimicrobial agent, which in some embodiments can comprise, consist essentially of, or consist of a nucleotide or nucleoside analogue, and/or is an anti-retroviral agent.

In some embodiments, the second lipid bilayer is derived from a virus, optionally a coronavirus, further optionally a SARS-CoV-2 virus.

In some embodiments, the presently disclosed subject matter provides pharmaceutical compositions comprising the exosome-derived nanoparticles disclosed herein and a pharmaceutically-acceptable vehicle, carrier, or excipient.

In some embodiments of the presently disclosed subject matter, a pharmaceutical composition is provided that comprises an exosomal composition disclosed herein and a pharmaceutical vehicle, carrier, or excipient. In some embodiments, the pharmaceutical composition is pharmaceutically-acceptable in humans. Also, as described further below, the pharmaceutical composition can be formulated as a therapeutic composition for delivery to a subject in some embodiments.

A pharmaceutical composition as described herein in some embodiments comprises a composition that includes pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and PCT

International Patent Application Publication No. WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of capsules, tablets or lozenges formulated in conventional manner.

Various liquid and powder formulations can also be prepared by conventional methods for inhalation into the lungs of the subject to be treated or for intranasal administration into the nose and sinus cavities of a subject to be treated . For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the present invention and a physiologically-acceptable excipient is infused.

Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically-acceptable oil base, such as an ester of a long chain fatty acid, (e.g., ethyl oleate).

In addition to the formulations described above, the exosomal therapeutic agents of the present invention can also be formulated as rectal compositions, such as suppositories or retention enemas, e.g., containing conventional suppository bases such as cocoa butter or other glycerides. Further, the exosomal compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

In some embodiments of the presently-disclosed subject matter, the exosomal compositions of the presently-disclosed subject matter specifically bind to a target cell or tissue. Applicants have discovered that exosomes released from different types of cells (i.e., derived from different cells) with different levels of activation (e.g. proliferating vs. non-proliferating) exhibit tissue- and/or cell-specific in vivo tropism, which can advantageously be utilized to direct the exosomes and the exosomal compositions to a specific cell or tissue. For example, in some embodiments, the exosome used to produce an exosomal composition of the presently-disclosed subject matter is derived from a T lymphocyte and specifically binds CD11b⁺Gr1⁺ myeloid cells.

Further provided, in some embodiments of the presently-disclosed subject matter, are methods for treating an inflammatory disorder. In some embodiments, a method for treating an inflammatory disorder is provided that comprises administering to a subject in need thereof an effective amount of an exosomal composition of the presently-disclosed subject matter. In some embodiments of the presently-disclosed methods of treating an inflammatory disorder, the therapeutic agent encapsulated by an exosome is a phytochemical agent and/or a Stat-3 inhibitor.

As used herein, the terms “treatment” or “treating” relate to any treatment of a condition of interest (e.g., an inflammatory disorder or a cancer), including but not limited to prophylactic treatment and therapeutic treatment. As such, the terms “treatment” or “treating” include, but are not limited to preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

As used herein, the term “inflammatory disorder” includes diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories. Exemplary inflammatory disorders include, but are not limited to atherosclerosis; arthritis; inflammation-promoted cancers; asthma; autoimmune uveitis; adoptive immune response; dermatitis; multiple sclerosis; diabetic complications; osteoporosis; Alzheimer' s disease; cerebral malaria; hemorrhagic fever; autoimmune disorders; and inflammatory bowel disease. In some embodiments, the inflammatory disorder is an autoimmune disorder that, in some embodiments, is selected from lupus, rheumatoid arthritis, and autoimmune encephalomyelitis.

In some embodiments, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject. It has been determined that the exosomal compositions of the presently-disclosed subject matter are particularly suitable for treating such disorders as those compositions are able to cross the blood-brain barrier and effectively be used to deliver the therapeutic agents (e.g., curcumin or JSI-124) to the brain of a subject.

For administration of a therapeutic composition as disclosed herein (e.g., an exosome encapsulating a therapeutic agent), conventional methods of extrapolating human dosage based on doses administered to a murine animal model can be carried out using the conversion factor for converting the mouse dosage to human dosage: Dose Human per kg=Dose Mouse per kg×12 (Freireich et al., 1966). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see e.g., U.S. Pat. No. 6,180,082).

Regardless of the route of administration, the compositions of the presently-disclosed subject matter are typically administered in amount effective to achieve the desired response. As such, the term “effective amount” is used herein to refer to an amount of the therapeutic composition (e.g., an exosome encapsulating a therapeutic agent, and a pharmaceutically vehicle, carrier, or excipient) sufficient to produce a measurable biological response (e.g., a decrease in inflammation). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. In some embodiments, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., 1997; Goodman et al., 1996; Ebadi, 1998; Katzung, 2001; Remington et al., 1975; Speight et al., 1997; Duch et al., 1998.

In some embodiments of the therapeutic methods disclosed herein, administering an exosomal composition of the presently-disclosed subject matter reduces an amount of an inflammatory cytokine in a subject. In some embodiments, the inflammatory cytokine can be interleukin-1β (IL-1β), tumor necrosis factor-alpha (TNF-α), or interleukin-6 (IL-6).

Various methods known to those skilled in the art can be used to determine a reduction in the amount of inflammatory cytokines in a subject. For example, in certain embodiments, the amounts of expression of an inflammatory cytokine in a subject can be determined by probing for mRNA of the gene encoding the inflammatory cytokine in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the presently-disclosed subject matter.

With further regard to determining levels of inflammatory cytokines in samples, mass spectrometry and/or immunoassay devices and methods can also be used to measure the inflammatory cytokines in samples, although other methods can also be used and are well known to those skilled in the art. See e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See e.g., U.S. Pat. Nos. 5,631,171; and 5,955,377, each of which is hereby incorporated by reference in its entirety.

Any suitable immunoassay can be utilized, for example, enzyme-linked immunoassays (ELISA), radioimmunoassays (RIAs), competitive binding assays, and the like. Specific immunological binding of the antibody to the inflammatory molecule can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The use of immobilized antibodies or fragments thereof specific for the inflammatory molecules is also contemplated by the present invention. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

Mass spectrometry (MS) analysis can be used, either alone or in combination with other methods (e.g., immunoassays), to determine the presence and/or quantity of an inflammatory molecule in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electrospray ionization MS

(ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as, for example, triple quadropole mass spectrometers. Methods for utilizing MS analysis to detect the presence and quantity of peptides, such as inflammatory cytokines, in biological samples are known in the art. See e.g., U.S. Pat. Nos. 6,925,389; 6,989,100; and 6,890,763 for further guidance, each of which are incorporated herein by this reference.

With still further regard to the various therapeutic methods described herein, although certain embodiments of the methods disclosed herein only call for a qualitative assessment (e.g., the presence or absence of the expression of an inflammatory cytokine in a subject), other embodiments of the methods call for a quantitative assessment (e.g., an amount of increase in the level of an inflammatory cytokine in a subject). Such quantitative assessments can be made, for example, using one of the above mentioned methods, as will be understood by those skilled in the art.

The skilled artisan will also understand that measuring a reduction in the amount of a certain feature (e.g., cytokine levels) or an improvement in a certain feature (e.g., inflammation) in a subject is a statistical analysis. For example, a reduction in an amount of inflammatory cytokines in a subject can be compared to control level of inflammatory cytokines, and an amount of inflammatory cytokines of less than or equal to the control level can be indicative of a reduction in the amount of inflammatory cytokines, as evidenced by a level of statistical significance. Statistical significance is often determined by comparing two or more populations, and determining a confidence interval and/or a p value. See e.g., Dowdy & Wearden, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

Further provided, in some embodiments, are methods for inhibiting immune responses against microbial antigens, optionally immune responses against viral antigens, in subjects in need thereof. In some embodiments, the methods comprise, consist essentially of, or consist of administering to a subject in need thereof a composition comprising a plurality of plant-derived exosome-like particles, wherein the plurality of plant-derived exosome-like particles is present in the composition in amounts sufficient to inhibit the immune response against the microbial, optionally virus, antigen in the subject.

Further provided, in some embodiments, are methods for inhibiting development of septic shock in subjects in need thereof. In some embodiments, the methods comprise, consist essentially of, or consist of administering to the subject a composition comprising a plurality of plant-derived exosome-like particles, wherein the plurality of plant-derived exosome-like particles are present in the composition in an amount and the administering is via a route sufficient to inhibit development of septic shock in the subject.

Further provided, in some embodiments, are methods for inhibiting development of cytokine storm in subjects in need thereof. In some embodiments, the presently disclosed method comprise, consist essentially of, or consist of administering to a subject in need thereof a composition comprising a plurality of plant-derived exosome-like particles, wherein the plurality of plant-derived exosome-like particles are present in the composition in an amount and the administering is via a route sufficient to inhibit development of cytokine storm in the subject.

Further provided, in some embodiments, are methods for treating microbial, in some embodiments viral, infections in subjects. In some embodiments, the methods related to treating viral infections, optionally coronavirus infections, further optionally SARS-CoV-2 infections. In some embodiments, the methods comprise, consist essentially of, or consist of administering to a subject in need thereof an effective amount of a composition and/or a pharmaceutical composition as disclosed herein.

Further provided, in some embodiments, are methods for inhibiting a SARS-CoV-2 induced cytopathogenic effect (CPE) in subjects in need thereof. As used herein, the term “cytopathogenic effect” (also referred to as “cytopathic effect”) refers to structural changes in host cells that result by viral infections. CPE includes, but is not limited to lysis of the host cell and/or cell death without lysis due to replication blockade. In some embodiments, the methods comprise, consist essentially of, or consist of administering to a subject in need thereof an effective amount of a composition and/or a pharmaceutical composition as disclosed herein in an amount and via a route sufficient to inhibit SARS-CoV-2-induced CPE in the subject. In some embodiments, the composition and/or the pharmaceutical composition comprises an miR396a species, an miR-rL1-28 species, or any combination thereof. Thus, in some embodiments the instant method comprises, consists essentially of, or consists of administering to a subject in need thereof an effective amount of a composition and/or a pharmaceutical composition comprising a ginger exosome-like nanoparticle (GELN), a garlic exosome-like particle (G-Exo), a turmeric exosome-like particle (T-Exo), a lemon (L-exo) exosome-like particle, or any combination thereof comprising an miRNA aly-miR396a-5p in an amount and via a route sufficient to inhibit SARS-CoV-2-induced CPE in the subject. It is noted, however, that other miRNAs can be employed to target SARS-CoV-2 gene products, including but not limited to an miR396a species, an miR-rL1-28 species, or any combination thereof.

In some embodiments, the compositions of the presently disclosed subject matter are targeted to cells, tissues, and/or organs of interest in subjects. In some embodiments, the methods comprise, consist essentially of, or consist of administering to a subject in need thereof an effective amount of a composition and/or a pharmaceutical composition as disclosed herein, wherein the composition and/or the pharmaceutical composition comprises a targeting molecule that targets a therapeutic molecule to the cell, tissue, or organ of interest. In some embodiments, the targeting molecule is a component of the exosome-like particle, which in some embodiments can be a plant-derived exosome-like particles. In some embodiments, the plurality of plant-derived exosome-like particles are garlic-derived exosomes (G-Exo), turmeric-derived exosomes (T-exo), lemon-derived exosomes (L-Exo), or any combination thereof. In some embodiments, the composition is administered to the subject by inhalation and/or insufflation, optionally where in inhalation and/or insufflation occurs through the nasal cavity. In some embodiments, the microbial antigen is a virus antigen, optionally a coronavirus antigen, further optionally an antigen derived from a SARS-CoV-2 virus.

In some embodiments, one or more antimicrobial and/or antiviral treatments and/or one or more immunosuppressive treatments are also administered to the subject. The choice of antimicrobial and/or antiviral and/or immunosuppressive treatment(s) can in some embodiments depend on the microbial species at issue, and is within the skill of one of ordinary skill in the art to identify and determine, as appropriate.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Materials and Methods for the Examples

Cell culture. The mouse C57BL/6 lung carcinoma LLC1 and macrophage cell lines, monkey kidney Vero E6 cells, and human alveolar basal epithelial A549 and monocytic U937 cell lines (American Type Culture Collection, Rockville, Md., United States of America) were grown at 37° C. in 5% CO₂ in Dulbecco's Modified Eagle's medium (DMEM, Life Technologies Corporation, Carlsbad, Calif., United States of America) and RPMI 1640 Medium (Life Technologies), respectively. U937 cells were induced to differentiate into macrophages using phorbol-12-myristate-13-acetate (PMA; Sigma-Aldrich Corporation,. St. Louis, United States of America) at 10 ng/ml for 5-7 days prior to using in studies. Media were supplemented with 10% heat-inactivated fetal bovine serum (FBS), 100 U/mL penicillin, and 100 μg/mL streptomycin.

Virus Infection. SARS-CoV-2 strain USA-WA1/2020 isolate (Catalog No. NR-52281; BEI Resources, Manassas, Va., United States of America) was amplified in Vero E6 cells. Amplified stock virus was stored at −80° C. until used. For infection, cells were incubated with virus for one hour in a CO₂ incubator at 37° C., then washed once with PBS. The cells were replenished with media. Virus titration was done by overlaying cells with Avicel overlay media (1% Avicel in DMEM with 10% FBS) and stained three days post infection with crystal violet staining solution (1% crystal violet, 2% paraformaldehyde, 25% ethanol) for 4 hours. Virus titers were determined as median tissue culture infectious dose (TCID₅₀)/mL in confluent cells in 96-well microtiter plates (Cinatl et al., 2003). Experiments were performed with three replicates per treatment. Experimental procedures with SARS-CoV2 virus were approved by Institutional Biosafety Committee of the University of Louisville, Louisville, Kentucky, United States of America. All processing of virus were performed in University of Louisville Center for Predictive Medicine, which has state-of-the-art facilities for BSL-3 biocontainment research and in accordance with relevant guidelines and regulations by Institutional Biosafety Committee of University of Louisville and United States Centers for Disease Control and Prevention (Interim Guidelines for Collecting, Handling, and Testing Clinical Specimens for COVID-19).

Mice. 8- to 12-week-old male specific pathogen-free (SPF) C57BL/6 mice were purchased from the Jackson Laboratory (Bar Harbor, Me., United States of America) and housed under specific pathogen-free conditions. Animal care was performed following the Institute for Laboratory Animal Research (ILAR) guidelines and all animal experiments were done in accordance with protocols approved by the University of Louisville Institutional Animal Care and Use Committee (Louisville, Ky., United States of America).

Plasmid construction. Nsp12 and S protein genes of SARS2 virus were amplified from plasmids pGBW-m4046955 (Plasmid #145616) and pGBW-m4046887 (Plasmid #145730), respectively (both from Addgene, Watertown, Mass., United States of America), and cloned into plasmid pAcGFP1-C1 (Plasmid #121046; Addgene) using the NEBUILDER® HiFi DNA Assembly Cloning Kit (Cat No. E5520S; New England Biolabs, Ipswich, Mass., United States of America) following the manufacturer's instructions. Plasmid pAcGFP1-C1 was linearized by double digestion of Kpn I and BamH I. The primers used to generate construction are listed in Table 3. Positive colonies were confirmed by PCR and Sanger sequencing.

Isolation of Lung Macrophage in Mice. Lung specimen from mice were thoroughly dissected and gently pressed through nylon cell strainers (70 μm in diameter; Fisher Scientific, Waltham, Mass., United States of America) to obtain single-cell suspensions in RPMI-1640 containing 5% FBS. The cell pellet was resuspended in 40% Percoll and layered onto 70% Percoll in RPMI-1640 with lx Hanks' Balanced Salt Solution (HBSS; Thermo Fisher, Waltham, Mass., United States of America). After Percoll gradient centrifugation, the layer in the interface between the two Percoll concentrations was collected and washed with PBS. Erythrocytes in the cell suspensions were then removed using Ammonium-Chloride-Potassium (ACK) lysing buffer (0.15 M NH₄Cl, 10 mM KHCO₃, 0.1 mM EDTA). The cells were allowed to adhere to the tissue culture plate for 24 hours at 37° C. Nonadherent cells were removed by gently washing plates three times with warm PBS. At this point, the adhering cells were greater than 90% macrophages.

Isolation and purification of exosomes from cell culture medium. 1×10⁷ lung A549 or LLC1 cells were grown in 10 ml of DMEM supplemented with 10% heat-inactivated extracellular vesicle (EV)-depleted FBS, 100 U/mL penicillin, and 100μg/mL streptomycin at 37° C. in 5% CO₂ for 72 hours. Extracellular vesicle (EV)-depleted FBS was prepared by ultracentrifugation overnight. The culture medium was collected and centrifuged at 1,000 g for 10 minutes, 2,000 g for 20 minutes, 4,000 g for 30 minutes, and 10,000 g for 1 hour with the supernatant being retained each time. The exosomes were collected by centrifuging the samples at 100,000 g for at least 2 h at 4° C. and further purified on a sucrose gradient (8, 30, 45, and 60% sucrose in 20 mM HEPES, 20 mM Tris-Cl, pH 7.2) followed by two PBS washes. Size distribution and concentration of exosomes were analyzed at a flow rate of 0.03 mL per min using a Zetasizer Nano ZS (Malvern Instrument, United Kingdom) and NanoSight NS300 (Westborough, Mass., United States of America), respectively. Data have been submitted to the EV-TRACK knowledgebase (EV-TRACK ID: EV210123; Consortium et al., 2017).

Isolation, purification, and electron microscopy of ginger exosome-like nanoparticles (GELNs). Peeled Hawaiian ginger roots (Simply ginger, PLU #4612), were used for isolation and purification of ELNs using a previously described method (see e.g., PCT International Patent Application Publication No. WO 2019/104242; Teng et al., 2018). Briefly, peeled plants were homogenized in a high-speed blender for 1 minute. The juice was collected after net filtration. The supernatant was collected after centrifugation at 1,000 g for 10 minutes, 2,000 g for 20 minutes, 4,000 g for 30 minutes, and 10,000 g for 1 hour. The pellets of the plant nanoparticles were spun down at 100,000 g for 1.5 hour at 4° C. The isolated exosomes were further purified in a sucrose gradient (8, 30, 45, and 60% sucrose in 20 mM Tri-Cl, pH 7.2) followed by centrifugation at 100,000 g for 1.5 hour at 4° C. Purified GELNs were fixed in 2% paraformaldehyde and imaged using a Zeiss EM 900 electron microscope as described in PCT International Patent Application Publication No. WO 2019/104242 (see also Mu et al., 2014). Size distribution and concentration of the GELNs was analyzed at a flow rate of 0.03 ml per min using a Zetasizer Nano ZS (Malvern Instrument, United Kingdom).

Extraction and purification of lemon-derived exosome-like nanoparticles (L-Exo) from lemon fruit. Lemons were purchased from a local Sam's Club market. L-Exo extraction and purification was performed as described previously (Teng et al., 2018). Isolated L-Exo were resuspended in ice-cold 1× PBS and further purified by sucrose gradient ultracentrifuge (8, 30, 45, and 60% sucrose in 20 mM Tris-HCl, pH 7.2). The main band located at the 30%-45% sucrose interface was collected. All centrifugation steps were performed at 4° C. LELN size distribution and quantity were determined using a Zetasizer Nano ZS (Malvern Instrument, United Kingdom).

RNA extraction. Total RNA containing miRNA was isolated from ELNs and murine tissue using a miRNeasy mini kit (Qiagen Inc., Germantown, Maryland, United States of America) according to the manufacturer's instructions. In brief, 50 mg of plant derived ELNs or tissue or culture cells was disrupted in QIAzol Lysis Reagent. Tissue was homogenized using a tissue grinder before disruption. After mixing the homogenate with 140 μl of chloroform, the homogenate was centrifuged. The upper aqueous phase was mixed with 1.5 volumes of ethanol and then loaded into RNeasy spin column. Flow through the column was discarded and the column was washed with RWT and RPE, respectively. Total RNA was eluted with RNase-free water. Bacterial mRNA was isolated using the RibPure Bacteria and MICROBExpress kits (Thermo Fisher Scientific) according to the manufacturer's instructions. The quality and quantity of the isolated RNA was analyzed using a NanoDrop spectrophotometer and Agilent Bioanalyzer.

Preparation and characterization of GELN nanovectors (GNVs) and packaging ELN RNAs in GNVs. The GELN-derived lipids were extracted with chloroform and dried under vacuum. To generate GNVs, 200 nmol of GELN-derived lipid was suspended in 200-400 μl of 155 mM NaCl with or without 10 μg of ELN-derived RNA. After UV irradiation at 500 mJ/cm² in a Spectrolinker (Spectronic Corp.) and a bath sonication (FS60 bath sonicator, Fisher Scientific) for 30 minutes, the pelleted particles were collected by centrifugation at 100,000 g for 1 hour at 4° C. (see PCT International Patent Application Publication No. WO 2019/104242; Wang et al., 2013; Teng et al., 2016). RNA encapsulation efficiency in GNVs was determined as described in PCT International Patent Application Publication No. WO 2019/104242 (see also Teng et al., 2016).

Distribution in the mouse respiratory tract with intratracheal intubation. Mice were anesthetized with a 2-3% isoflurane/oxygen mixture in an anesthesia induction chamber. Mice were secured to the intubation platform. Direct laryngoscopy using an otoscope fitted with a 2.0 mm speculum was used to visualize the glottis following intubation using a 20 G intravenous catheter as an endotracheal tube. After confirmation of intubation was established using tubing containing a colored dye, exosomes or GNVs (5×10⁸/kg body weight, n=5 per group) were dispensed into the lung in a single fluid motion. The needle/catheter was removed and the mouse allowed to recover from anesthesia.

Exosome and GNVs distribution in vivo. Intratracheally administered exosomes or GNVs labelled with DiR dye (5×10⁸/kg, body weight, n=5 per group) were visualized in the lung and other organs of C57BL/6 mice using an Odyssey CLx Imaging System (LI-COR Biosciences, Lincoln, Nebr., United States of America).

Labeling exosomes and GNV vectors with PKH26. Exosomes and GNVs were labeled using PKH26 Fluorescent Cell Linker Kits (Sigma-Aldrich). GNVs were suspended in 250 μl of Diluent C with 5 μl of PKH26 mixed with 250 μl of dye solution for 20 minutes at room temperature and subsequently incubated with an equal volume of 1% bovine serum albumin (BSA) for 1 minute at 22° C. After centrifugation for 1 hour at 100,000 g at 4° C., 20 μl of resuspended labeled GNVs were loaded on a slide for assessment of viability using confocal microscopy (Nikon Americas Inc., Melville, N.Y., United States of America).

Quantitative Real-Time PCR (qPCR) analysis of miRNA and mRNA expression. The quantity of mature miRNAs was determined by quantitative real-time PCR (qPCR) using a miScript II RT kit (Qiagen) and miScript SYBR Green PCR Kit (Qiagen) with Qiagen 3′ universal primers. The 5′ specific miRNA primers used are listed in Table 3. For analysis of gene mRNA expression, 1 μg of total RNA was reverse transcribed by SuperScript III reverse transcriptase (Invitrogen Corporation, Carlsbad, Calif., United States of America) and quantitation was performed using SSOADVANCED™ Universal SYBR Green Supermix (BioRad Laboratories, Hercules, Calif., United States of America) with primers listed in Table 3 with SSOADVANCED™ Universal SYBR Green Supermix (BioRad). qPCR was run using the BioRad CFX96 qPCR System with each reaction run in triplicate. Analysis and fold-changes were determined using the comparative threshold cycle (Cₜ) method. After normalizing with an internal control glyceraldehyde 3-phosphate dehydrogenase (GAPDH) expression, the change of target gene in miRNA or mRNA expression was calculated as fold-change (i.e., relative to PBS or vehicle-treated (control)).

Flow cytometry. After perfusion via the inferior vena cava with perfusion buffer (Ca2⁺-Mg2⁺-free HBSS containing 0.5 mM EGTA, 10 mM HEPES and 4.2 mM NaHCO₃; pH 7.2), lung tissue from mice was incubated in RPMI supplemented with 15 mM HEPES and 300 units/ml collagenase type VIII (Sigma) for 1 hour with gentle shaking. After this the tissue was gently pressed through nylon cell strainers (70 μm in diameter, Fisher Scientific) to obtain single-cell suspensions in RPMI-1640 containing 5% FBS. Lung leukocytes were isolated from the interface of 40%/80% colloidal silica particle (Percoll) gradient and washed twice. Erythrocytes in liver and spleen-cell suspensions were then removed using ammonium-chloride-potassium (ACK) lysing buffer (0.15 M NH₄Cl, 10 mM KHCO₃, 0.1 mM EDTA). Washed cells were stained for 1 hour or overnight at 4° C. with the appropriate fluorochrome-conjugated antibodies in PBS with 2% FBS.

To detect intracellular antigens, washed cells were incubated in diluted Fixation/Permeabilization solution (eBioscience Cat #00-5123) at 4° C. for 30 min. Characterization and phenotyping of the various lymphocytes subsets from lung were performed by flow cytometry using antibodies against F4/80 (eBioscience Cat. No. 17-4801-82), Gr-1 (Cat. No. 11-9668-82; eBioscience Inc, San Diego, Calif., United States of America), CD-11b (eBioscience Cat. No. 11-0112-41), TNFα (eBioscience Cat. No. 11-7321-82), IL-1α (eBioscience Cat. No. 50-111-17), IL-1β (eBioscience Cat. No. 50-100-10), IL-6 and EpCAM (eBioscience Cat. No. 11-5791-82) at a 1:200 dilution with PBS/2% FBS for 1 h on ice. Annexin V-FITC was applied to detect non-viable cells and propidium iodide (PI) staining was used to distinguish apoptotic cells from necrotic and living cells. Data were acquired on a BD FACS CANTO™ (BD Biosciences, San Jose, Calif., United States of America) and were analyzed using FlowJo software (Tree Star Inc., Ashland, Oreg., United States of America). Numbers above bracketed lines or boxes in FACS figures indicate percent of positive stained cells and the results of cells stained with a isotype-matched control antibody are shown in gray color.

Mouse cytokine array. To investigate the level of the cytokines, lung tissue extracts were prepared in modified radioimmunoprecipitation assay (RIPA) buffer (Sigma) with the addition of protease and phosphatase inhibitors (Roche). Cytokine proteins were analyzed with a Proteome Profiler Mouse XL Cytokine Array Kit (R&D Systems, ARY028). Profiles of mean spot pixel density were created using a transmission-mode scanner and quantification of the spot intensity in the arrays was conducted with background subtraction using image analysis MasterPlex QT software (MiraiBio Group, Ltd).

Western blotting. The mice or cells were treated as indicated in individual Figure legends and tissue or cells were harvested in RIPA buffer with the addition of a protease inhibitor cocktail (Roche). Proteins of lysates were separated by 10% SDS-PAGE and transferred to Odyssey nitrocellulose membranes (LI-COR). Dual color precision protein MW markers (BioRad) were separated in parallel. Antibodies were purchased as follows: anti-Spike (S) of SARS-CoV-2 (Cat. No. GTX632604) from GeneTex (Irvine, Calif., United States of America); anti-CD63 (Cat. No. 143902) from Biolegend (San Diego, Calif., United States of America); anti-p-NF-κB p65 (Cat. No. 3031S), anti-p-AKT (Cat. No. 9271S), anti-p-JNK (Cat. No. 9251S), anti-p-ERK1/2 (Cat. No. 4370), and anti-p-p38 MAPK (Cat. No. 4511) from Cell Signaling Technology (Danvers, Mass., United States of America); anti-NF-κB p65 (Cat. No. 610869) from BD Biosciences (San Jose, Calif., United States of America); anti-p-IKKβ (Cat. No. ab59195) and anti-p-PI3K (Cat. No. ab182651) from Abcam (Cambridge, United Kingdom); anti-p-IκBα (Cat. No. sc-8404) and anti-GAPDH (Cat. No. sc-47724) from Santa Cruz Biotechnology (Santa Cruz, Calif., United States of America). The secondary antibodies conjugated to ALEXA FLUOR® 790 were purchased from Invitrogen (Eugene, Oreg., United States of America). The membranes were incubated with primary antibodies above at a dilution of 1:1,000 with PBST (PBS, 0.1% Tween 20) for 1 hour at room temperature. After the secondary antibody incubation at a dilution of 1:10,000 with PBST (PBS, 0.1% Tween 20) for 1 hour at room temperature, the bands were visualized and analyzed using an Odyssey Imager (LiCor Inc, Lincoln, Nebr., United States of America).

Transient transfection. A549 or LCC1 cells were grown to 70% confluency in tissue culture plates in antibiotic-free DMEM supplemented with 5% FBS. Cells were transfected with 200 pmoles miRNA or 10 μg plasmid/well using 30 μl of RNAiMAX or Lipofectamine 3000 (Invitrogen) in antibiotic-free medium and incubated for 48-72 hours. As a control, cells were transfected with scramble control miRNA (Ambion, Inc., Austin Tex., United States of America) or empty plasmid vector. Expression plasmids used for transfections are listed in Table 1. RNA and protein lysates were prepared for qPCR and Western blot analysis.

Immunoblot analysis of viral production from transfected cells. Exosomes and A549 cells transiently transfected with viral plasmids were lysed with RIPA buffer. One μg of lysate was separated by 8-15% SDS-PAGE and transferred to nitrocellulose membranes (LI-COR) and blocked with 5% BSA in PBS at 4° C. overnight. Strep fusion protein and CD63 were visualized by StrepTactin-HRP conjugate (BioRad) and ALEXA FLUOR® fluorescent conjugated anti-CD63 antibody, respectively.

Preparation of GELN small RNA Libraries and sequencing. Small RNA libraries were generated with 100 ng of total RNA and QIAseq miRNA Library Kit (Qiagen) according to the manufacturer's instructions. Following reverse transcription, cDNA purification with QIAseq Beads and PCR amplification (16 cycles) with indices, libraries with approximately 180 bp were bead-purified and pre-sequencing QC was performed with an Agilent Bioanalyzer 2100. Equal amounts of libraries were pooled and sequenced on the Illumina HiSeq 2500 using the Illumina NextSeq Sequencing kit (Cat. No. FC-404-2005), followed by demultiplexing and fastq generation with CASAVA v1.8.4.

Raw fastq files were preprocessed and miRNA sequences were quantified using Qiagen's QIAseq miRNA quantification module (https://geneglobe.qiagen.com/us/). Briefly, 3′ adaptors were trimmed using cut adapt. Then, all miRNA sequences in the miRBase v21 were used for miRNA sequence quantification. Within the sRNABench pipeline, mapping was performed with bowtie (v0.12.9) and microRNA folding was predicted with RNA fold from the Vienna package (v2.1.6). To visualize miRNA-seq results with Waterfall and heatmap, low-expressed miRNAs (raw read count <10 in all samples) were removed. Then, miRNA expression levels were normalized using edgeR's TMM (trimmed mean of M values; Robinson & Oshlack, 2010). To generate a waterfall plot of potential differentially expressed miRNAs, a strict fold-change threshold (|log2 (fold change)|≥2) was employed. Heatmaps were generated using heatmap.2 function from the gplots R package (Warnes et al., 2009). Human and mouse miRNA-seq samples were downloaded from Sequence Read Archive (SRA; Leinonen et al., 2010) using accession numbers SRR12338616 and SRR7777390, respectively.

Predicting GELN miRNA targeting to SARS-CoV-2 mRNA. The nucleotide sequence of the SARS-CoV-2 genome (Accession No. NC_045512.2 of the GENBANK® biosequence database) was downloaded from the NCBI Nucleotide database. Viral mRNAs targeted by GELN miRNAs were identified by mapping the reverse complement of the miRNA seed sequence to the SARS-CoV-2 whole genome with the full-length 29,903 bp. Although 6 nucleotides (nt) are the minimum requirement and the 6-8 nt long seed sequence of the miRNA is sufficient to bind the target mRNA (Ellwanger et al., 2011), the enrichment analysis with 9-nt seed subsequences adopted a framework that utilized the 1st order Markov model (MM; Murphy et al., 2008). In this framework, the observed k-mer count in the 300 bp region of each bacterial mRNA was compared against the background count derived from the 1st order Markov model. A p-value was then calculated for each miRNA-mRNA pair to estimate the likelihood of having a functional pair. Once all p-values were calculated, false discovery rate (FDR) was obtained using the Benjamini-Hochberg method (Benjamini & Hochberg, 1995) for the multiple comparisons. The plots of miRNA distribution in SARS-CoV-2 genome were generated using a R4.0 programming environment. To determine if the seed sequences are present in human or mouse miRNA sequences, all of the human and mouse microRNA mature sequences (reference sequences) from the miRBase database v22 (Kozomara et al., 2019) were downloaded. Then, reference sequences that had a perfect match (no mismatches were allowed) to a seed sequence were identified.

Histological analysis. For hematoxylin and eosin (H&E) staining, tissues were fixed with buffered 10% formalin solution (SF93-20; Fisher Scientific, Fair Lawn, N.J., United States of America) overnight at 4° C. Dehydration was achieved by immersion in a graded ethanol series: 70%, 80%, 95%, and 100% ethanol for 40 minutes each. Tissues were embedded in paraffin and subsequently cut into ultra-thin slices (5 μm) using a microtome. The sections were deparaffinized with xylene (Fisher) and rehydrated with decreasing concentrations of ethanol and PBS. Tissue sections were then stained with H&E and slides were scanned with an Aperio ScanScope. For frozen sections, tissues were fixed with periodate-lysine-paraformaldehyde (PLP) and dehydrated with 30% sucrose in PBS at 4° C. overnight. The sections were incubated with anti-F4/80 (Cat. No. sc-25830; Santa Cruz), anti-Gr-1 (Cat. No. 108404; Biolegend), anti-CD-1 lb (Cat. No. 101204; Biolegend) and anti-EpCAM (Cat No. ab71916; Abcam) at a 1:100 dilution at 4° C. overnight. The signal was visualized with the secondary antibodies conjugated to ALEXA FLUOR® 488 or ALEXA FLUOR® 594 (Invitrogen) and nuclei were stained with 4′,6-diamidino-2-phenylindole dihydrochloride (DAPI). The slides were scanned using an Aperio ScanScope or visualized with a confocal laser scanning microscope (Nikon, Melville, N.Y., United States of America) as described in Teng et al., 2017.

Apoptosis analysis by TUNEL. Formalin-fixed mouse lung tissues were embedded in paraffin, sectioned, and place on glass slides. TUNEL was used to detect apoptosis in the sections according to the manufacturer's protocol. Tissue sections were analyzed to detect the localized green fluorescence (GFP) of apoptotic cells using a Roche In Situ Cell Death Detection Ki and blue fluorescence of cell nuclei using DAPI. The signal was visualized using confocal laser scanning microscopy (Nikon, Melville, N.Y., United States of America).

Enzyme-linked immunosorbent assay (ELISA). The cytokines in cell culture supernatants or mouse lung tissue were quantified using ELISA kits (eBioscience) according to the manufacturer's instructions. Briefly, a microtiter plate was coated with anti-mouse TNFα, IL-1α, IL-1β, and IL-6 antibody at 1:200 at 4° C. overnight. Excess binding sites were blocked with 200 μl of 1× ELISA/ELISPoT Diluent (eBioscience) for 1 hour at 22° C. After washing three times with PBS containing 0.05% Tween 20, the plate was incubated with detective antibody in blocking buffer for 1 hour at 22° C. After washing three times, avidin conjugated with horseradish peroxidase and substrate were each added sequentially for 1 hour and 30 minutes at 22° C., respectively. Absorbance at 405 nm was recorded using a microplate reader (Synergy HT, BioTek, Winooski, Vt., United States of America).

Isolation of Apoptotic Bodies. Apoptotic bodies (ABs) were isolated from culture supernatants as described in Masvekar et al., 2019. Briefly, cell culture medium was harvested and cells were removed by pelleting at 335 g for 10 minutes. To remove cell-debris, cell-free supernatants were centrifuged at 1,000 g for 10 minutes followed by another centrifugation at 2,000 g for 30 minutes to pellet ABs. Pelleted ABs were resuspended and washed with PBS.

Co-Immunoprecipitation (Co-IP) assay. To further confirm the interaction of Nsp12 and Nsp13, A549 cells were co-transfected with the plasmids of pAcGFP1-C-Nsp12-Flag and pLVX-Nsp13-2xStrep. 72 hours after transfection, Nsp13 was pulled down with Strep-Tactin XT magnetic beads in IP buffer (150 mM NaCl, 10 mM Tris-HCl (pH 7.4), 1 mM EDTA, 1 mM EGTA (pH 8.0), 0.2 mM sodium ortho-vanadate, 0.2 mM PMSF, 1% Triton X-100, 0.5% NP-40). 1 μg of Nsp12/Nsp13 complex was spotted onto nitrocellulose membranes (LI-COR) and blocked with 5% BSA in PBS at 4° C. overnight. Nsp12 fusion protein was visualized by rabbit anti-Flag antibody (Cat. No. SAB4301135; Sigma-Aldrich Corporation, St. Louis, Mo., United States of America) and ALEXA FLUOR® 790 conjugated anti-rabbit antibody (Invitrogen).

Nucleic acid unwinding assay. Helicase reactions were carried out in triplicate in 96-well plates. As for the helicase activity, 20 μM double strand DNA (dsDNA) (5′-AATGTCTGACGTAAAGCCTCTAAAATGTCTG-BHQ-3′; SEQ ID NO: 227; 5′-Cy3-CAGACATTTTAGAGG-3′; SEQ ID NO: 228) and 100 μM trap ssDNA (5′-CCTCTAAAATGTCTG-3′; SEQ ID NO: 229) was incubated with Nsp13 (100 nM) with or without Nsp12 in the reaction buffer (25 mM HEPES 7.5, 100 mM NaCl, 15 mM MgCl₂, 1 mM DTT, 0.1 mg/ml BSA) at 25° C. for 5 minutes. After adding ATP (2 mM) to initiate the helicase activity, the fluorescence value was recorded using a microplate reader with excitation wavelength set to 550 nm and emission wavelength set to 570 nm.

Cytopathic effects (CPE) of SARS-CoV-2. Vero e6 cells (passage 6) were seeded at a density of 2×10⁴ cells per well in a 96-well plate in DMEM supplemented with 10% heat-inactivated FBS, 100 U/mL penicillin, and 100 μg/mL streptomycin. After attachment of cells to the well bottoms, SARS-CoV-2 was diluted in DMEM to give a final concentration of 60 pfu/well for a MOI of 0.003. GELN miRNA (100 ng) packed in GNVs was added to the wells at dilutions from 1:1 to 1:100. Cells were observed for cytopathic effect (CPE) every day post-infection. On day 3 post-infection, after washing three times with PBS, the cells were collected in RIPA buffer or Trizol for virus protein and gene expression by western blot and qPCR analysis, respectively.

Statistical analysis. All statistical analyses were performed with SPSS 16.0 software. Data are presented as mean±standard deviation (SD). The significance of mean values between two groups was analyzed using the Student's t test. The differences between individual groups were analyzed by one- or two-way ANOVA test. The differences between percentages of CPE and signal positive cells in flow cytometer and confocal microscopy were analyzed with a chi-square test. The differences were considered significant when the p value was less than 0.05 or 0.01.

### Example 1

### Non-invasive Intranasal Administration of T-Exo and G-Exo Results in Uptake by Immune Cells

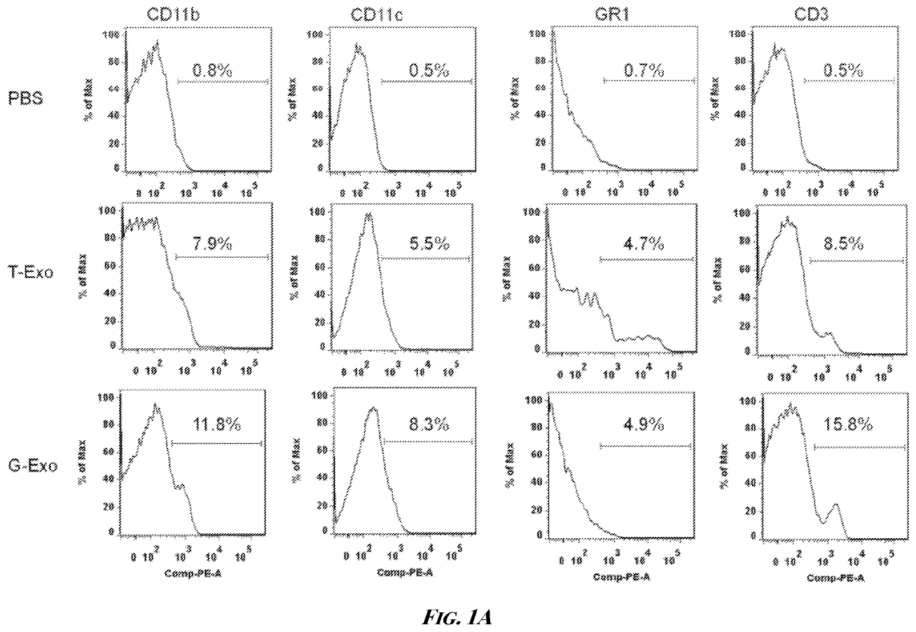

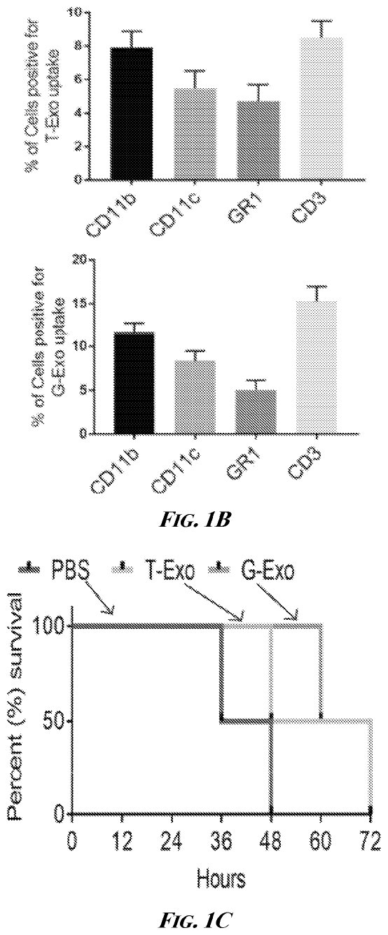

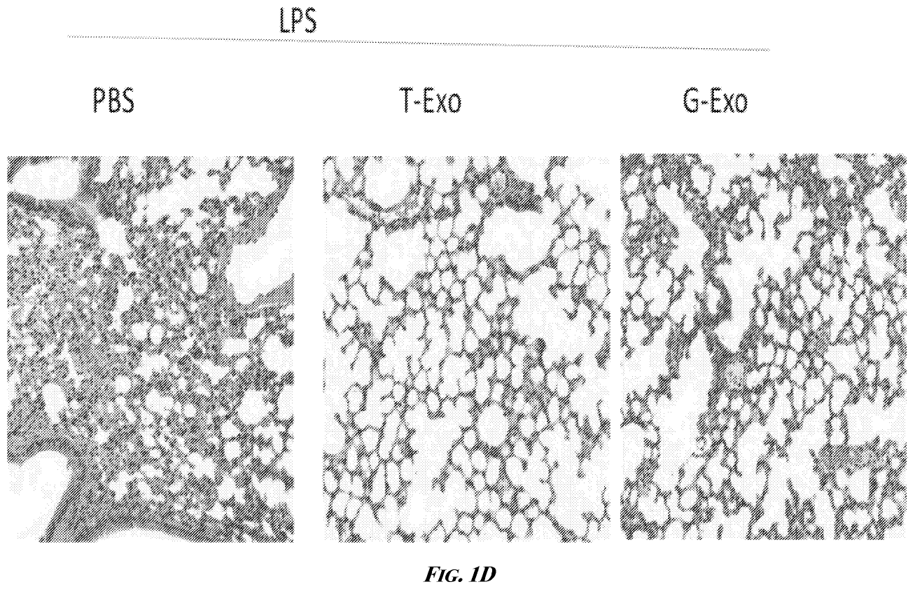

One of the features of LPS-induced septic shock is a robust increase of the number of immune cells that are sequestered in the lungs, leading to acute lung inflammation. Whether immune cells in the lung can take up G-Exo and T-Exo was first evaluated. A mouse model of LPS-induced acute lung injury model was used as set forth in Zhuang et al., 2016. Briefly, 1 hour after C57BL/6 mice (n=5) were injected with 18.5 mg LPS/kg of body weight (Sigma-Aldrich Chemical Co., St. Louis, Mo., United States of America) via an intraperitoneal route, mice were then treated with G-Exo or T-Exo (5×10⁷ exosomes per dose; 5 doses/day in total 20 μl PBS) via intranasal administration for three days. The last dose included PKH26-labelled T-Exo or G-Exo exosomes, and after 12 hours mice were sacrificed and organs were harvested for FACS analysis and confocal immune staining. The FACS data (see FIG. 1A) indicated that both T-Exo and G-Exo were taken up by CD3⁺ T cells, CD11b⁺ myeloid cells, CD11c dendritic cells, and Gr-1 neutrophils, with CD3⁺ T cells at the highest frequency in uptake. Confocal analysis results (see FIG. 1B) indicated that the majority of lung F4/80 macrophages took up T-Exo and G-Exo exosomes. This finding provided a foundation for G-Exo- and T-Exo-based cell type-specific delivery of therapeutic agents to the lung, including but not limited to various immune cells present in and/or locating to the lung.

### Example 2

### Intranasal Administration of T-Exo and G-Exo Protected Mice from LPS-Induced Septic Shock

Next, whether T-Exo and G-Exo provided beneficial effects on inhibition of LPS-induced septic shock was tested. Using the same approach as described above, the mortality data indicated that both T-Exo and G-Exo exosomes protected mice from LPS-induced septic shock as compared with PBS-treated mice, and also that G-Exo provided better protection against LPS-induced septic shock than T-Exo in this model (see FIG. 1C). This result was further supported by the fact that there were much fewer immune cells infiltrated into the lung and less lung damage subsequent to T-Exo and/or G-Exo exosome treatment (FIG. 1D).

### Example 3

### Preparation and Use of Coated Exosome-Like Nanoparticles

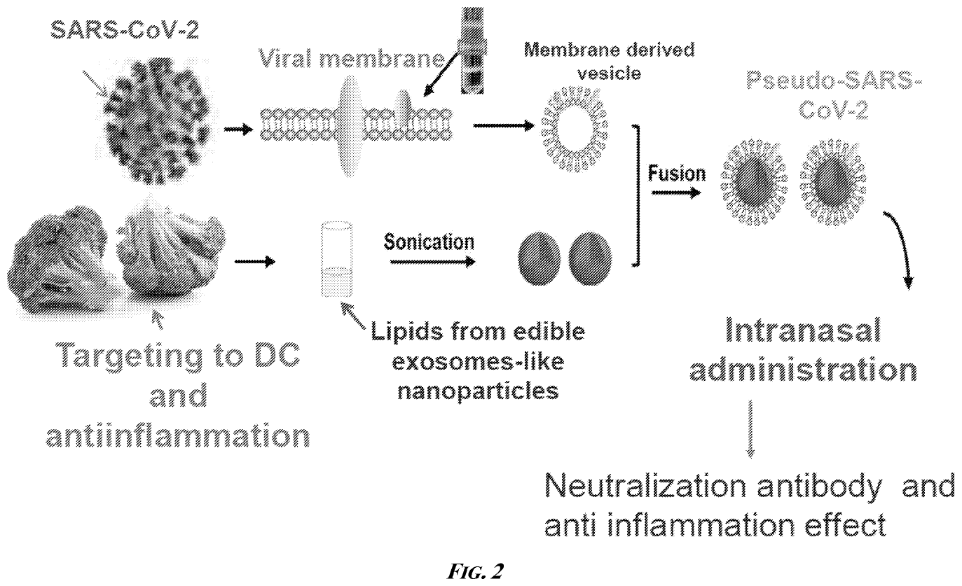

FIG. 2 provides an exemplary strategy for preparing a coated (e.g., encapsulated) exosome-like nanoparticle (e.g., a plant-derived exosome-like nanoparticle), wherein the exosome-like nanoparticle is coated by (e.g., enveloped by) a lipid bilayer that comprises a targeting molecule of interest. By way example and not limitation, FIG. 2 shows that a plant-derived exosome-like particle such as but not limited to a broccoli-derived exosome-like particle, a G-Exo, a T-Exo, an L-Exo, or indeed any exosome-like particle derived from any species (e.g., from a plant or animal, in some embodiments from a human) can be targeted to a target cell, tissue, or organ of interest (e.g., the lung or a cell present therein) by coating the exosome-like particle with a lipid bilayer that comprises a targeting molecule that is capable of targeting the exosome-like particle to the target cell, tissue, or organ of interest.

As shown in FIG. 2, lipid bilayers from SARS-CoV-2 virions can be isolated as membrane-derived vesicles, which by fusion with and/or by coating of an exosome-like particle derived from, for example, broccoli, can produce a pseudo-SARS-CoV-2 particle that targets the lungs. These pseudo-SARS-CoV-2 particles can then be administered to a subject in need thereof intranasally, thereby introducing the pseudo-SARS-CoV-2 particles to the lungs where they can accumulate. If the exosome-like particle itself comprises and/or is otherwise associated with a therapeutic molecule (e.g., encapsulates a therapeutic molecule), the exosome-like particle can be used to deliver the therapeutic molecule to the lungs for treatment of a disease, disorder, or condition of the lungs that includes, but is not limited to, a SARS-CoV-2 infection. It is noted, however, that even though a pseudo-SARS-CoV-2 particle is employed, the use of such a particle is not limited to treating a SARS-CoV-2 infection per se and can be used to treat any disease, disorder, or condition of the lungs for which an appropriate therapeutic molecule can be delivered.

### Example 4

### Lung Epithelial Derived Exosomes Containing SARS-COV-2 NSP12 and 13 Have a Synergistic Effect on the Induction of Inflammatory Cytokines In Vitro and In Vivo

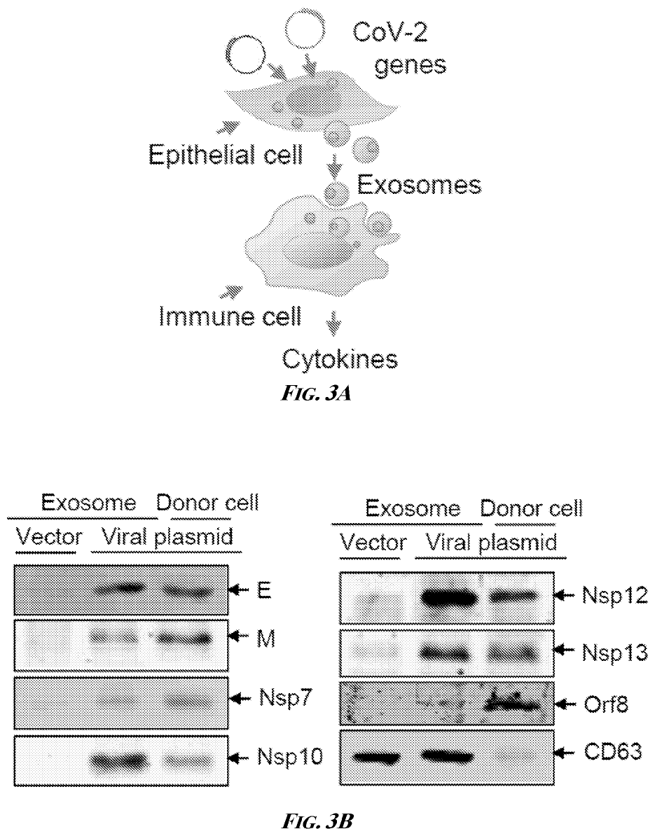

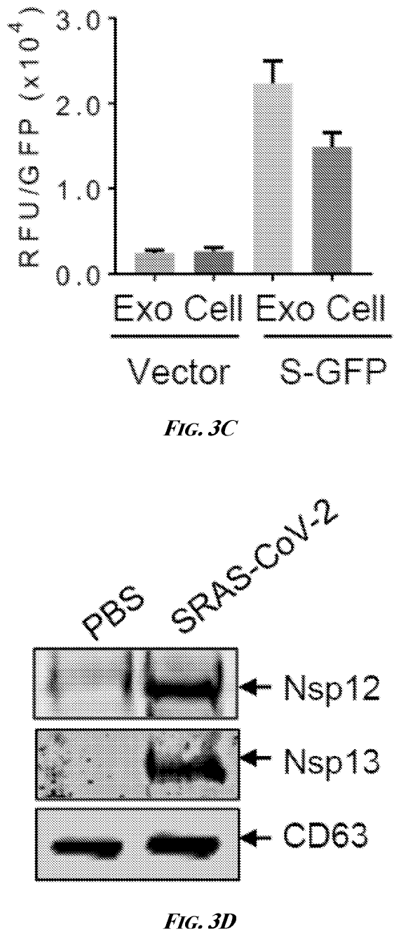

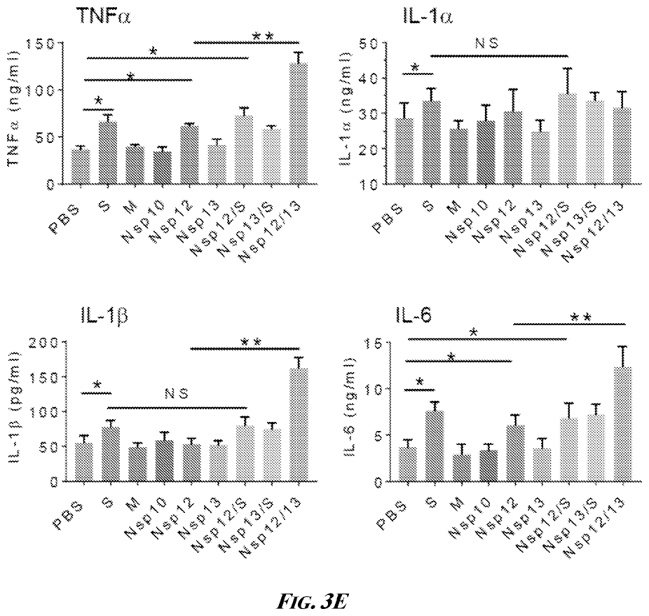

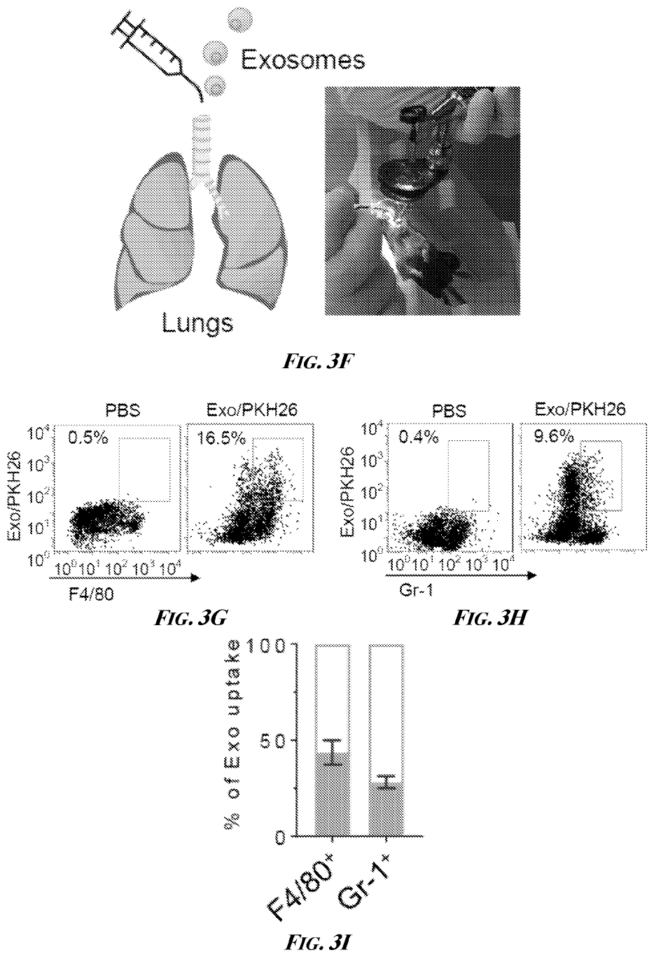

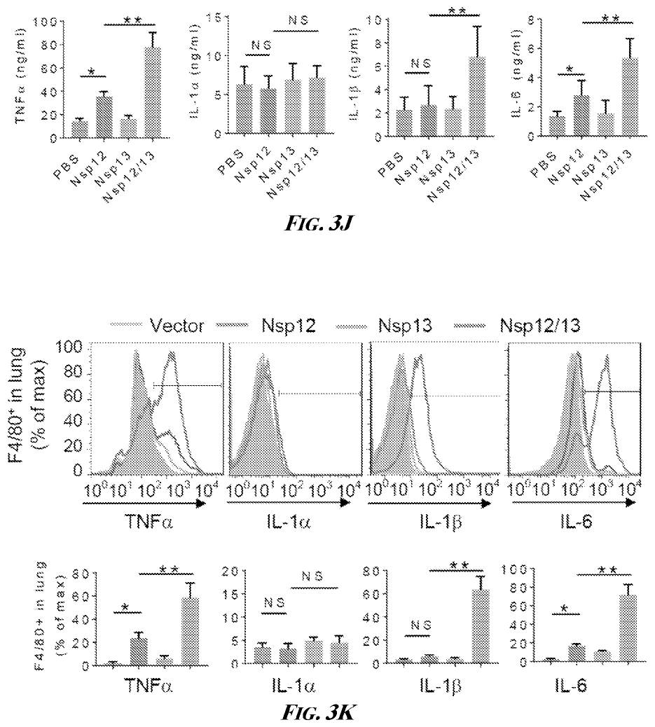

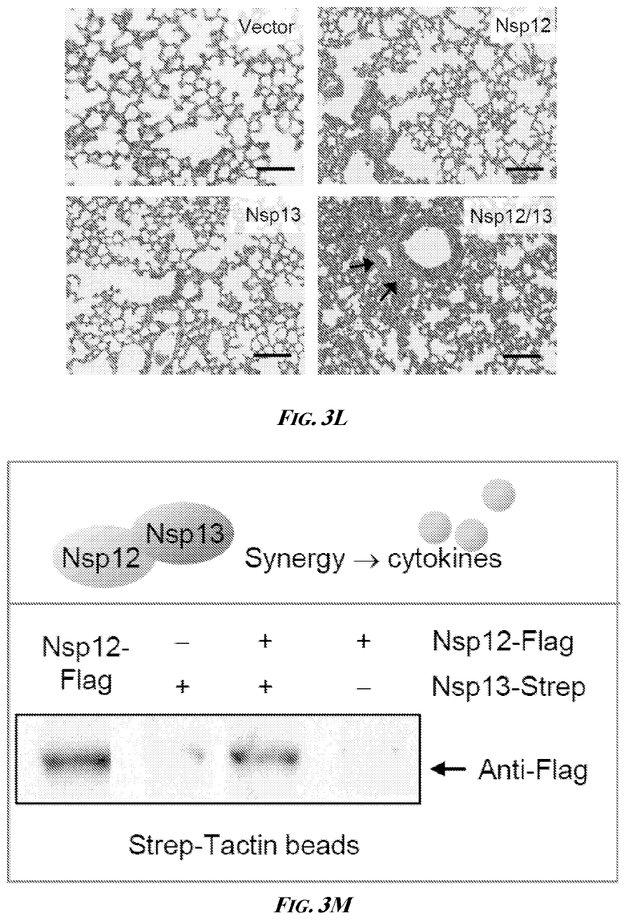

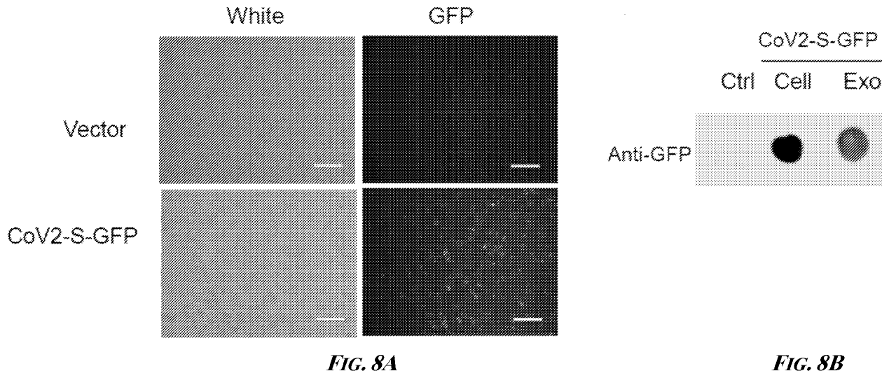

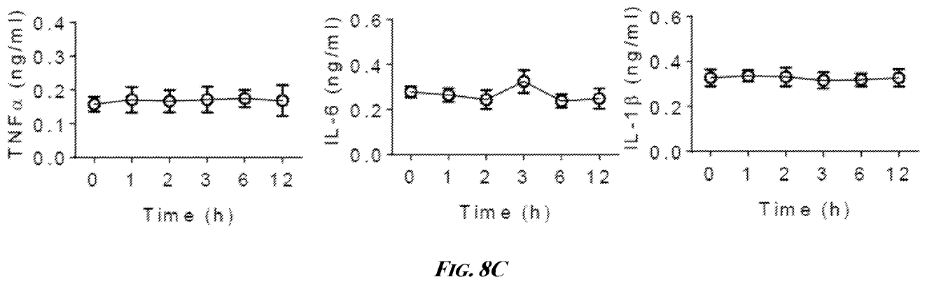

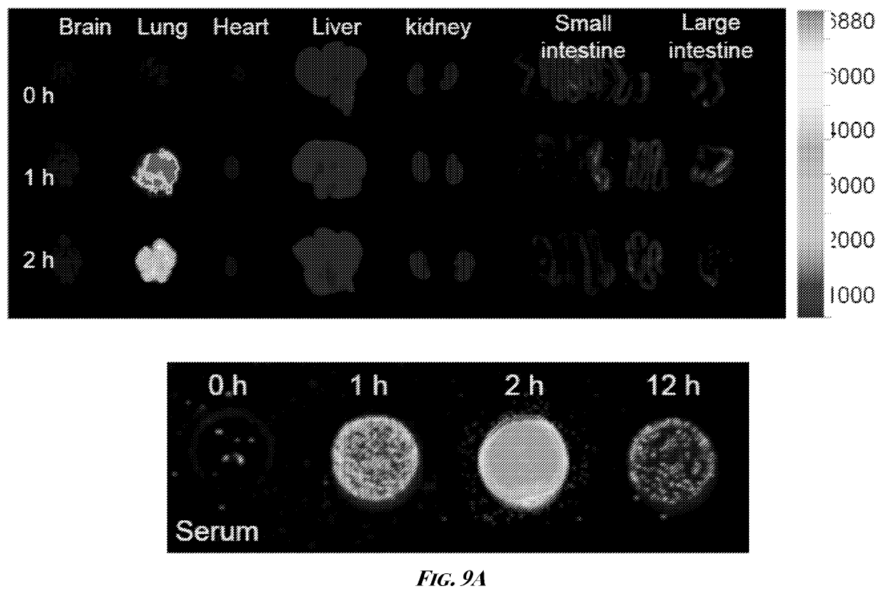

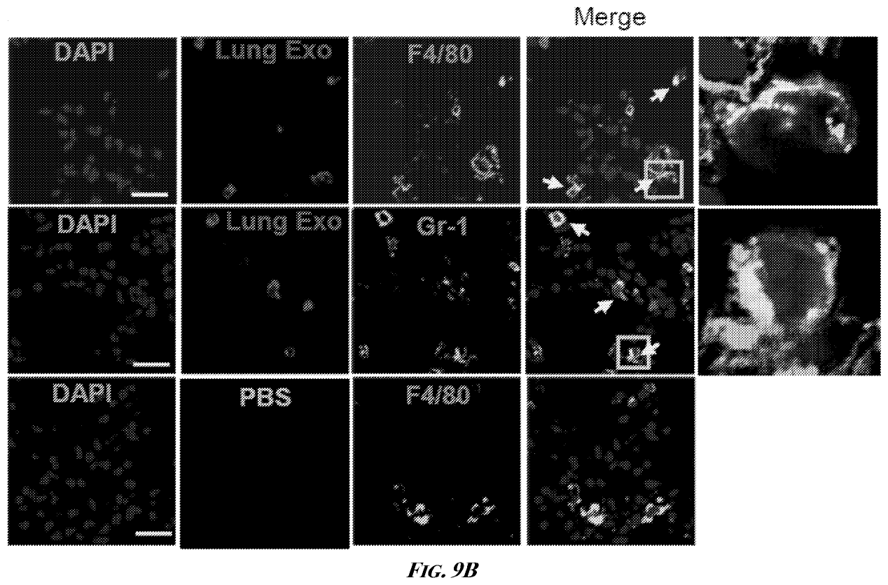

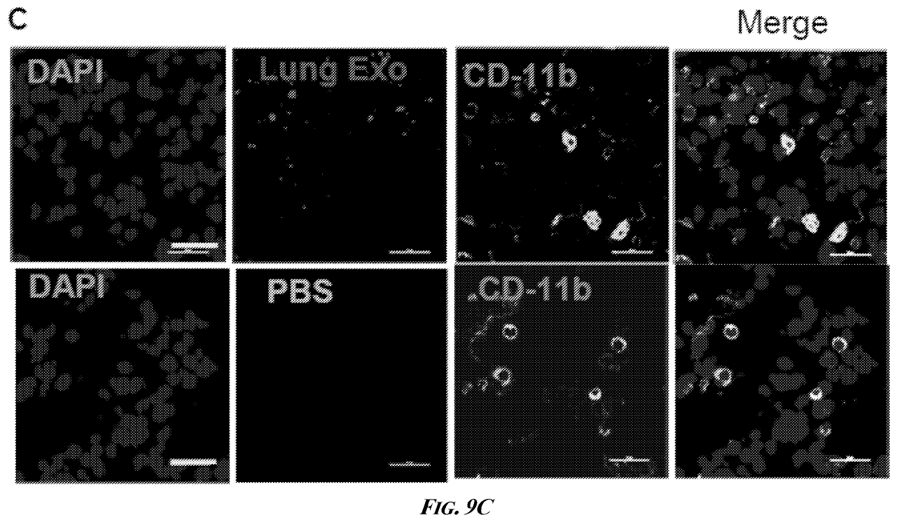

A growing number of reports suggest that infected cells employ exosome-mediated intercellular communication to induce inflammation (see e.g., Raab-Traub & Dittmer, 2017; Cantoni & Rossman, 2018; Chettimada et al., 2018; Higuchi et al., 2018; Pastuzyn et al., 2018; Santiana et al., 2018; Huang et al., 2020; Nahand et al., 2020; Velandia-Romero et al., 2020). Thus, whether exosomes released from lung epithelial cells expressing SARS-CoV-2 genes are loaded with the viral protein cargo was tested. A mammalian expression vector expressing viral genes encoding for the SARS-CoV-2, spike (S), envelope (E), matrix (M), Nsp7, Nsp10, Nsp12, Nsp13, or orf8, respectively were transfected into human lung epithelial A549 cells (see FIG. 3A and Table 1). Seventy-two hours after the transfection, exosomes released from the supernatants of cultured human lung epithelial A549 cells were isolated by differential centrifugation and confirmed by the exosome marker CD63 in a western blot assay (FIG. 3B). Using a StrepTactin-HRP conjugate, it was determined that all of viral proteins co-expressed with 2xStrep were successfully expressed in A549 cells; the cargo in A549 cell-released exosomes contained viral protein E, Nsp7, Nsp10, Nsp12, Nsp13, slight protein M, but not orf8 (FIG. 3B). In an independent experiment, it was also demonstrated that viral protein spike (S) fused with green fluorescent protein (GFP) in the cell-derived exosomes (see FIGS. 3C, 8A, and 8B).

To further confirm this result, Vero E2 cells were transfected with plasmids expressing Nsp12 or Nsp13 for three days. Exosomes in the medium were isolated and western blot analyses suggested that the exosomes released from Vero E2 cells contained viral Nsp12 and Nsp13 (see FIG. 3D).

Considering the lung epithelial cell exosomes can be taken up by lung macrophages, the impact of exosomes with viral protein on macrophage activation was evaluated. The levels of cytokines in the medium of human monocyte U937 cells were quantitatively analyzed with a standard enzyme-linked immunosorbent assay (ELISA). Interestingly, as an RNA polymerase, Nsp12 alone slightly induced tumor necrosis factor a (TNFα) and interleukin (IL)-6. Moreover, the synergistic effect of Nsp12 working with Nsp13 was observed where a dramatic induction of TNFα, IL-1β and IL-6 occurred when compared to Nsp12 alone, whereas protein M, Nsp13 alone, or Nsp10 did not (FIG. 3E). It was also determined that exosomes containing protein S induced TNFα, IL-1α, IL-1β and IL-6, but had no synergistic effect with Nsp12 or Nsp13 on the impact of cytokines (FIG. 3E). To further confirm the A549 exosomes mediated induction of inflammation cytokine TNFα as an example is viral Nsp12 and Nsp13 specific, U937 cells were exposed to A549 derived exosomes and ELISA analysis of cytokines suggested no significant influence of protein expression of TNFα from 1 hour up to 12 hours post exposure (FIG. 8C).

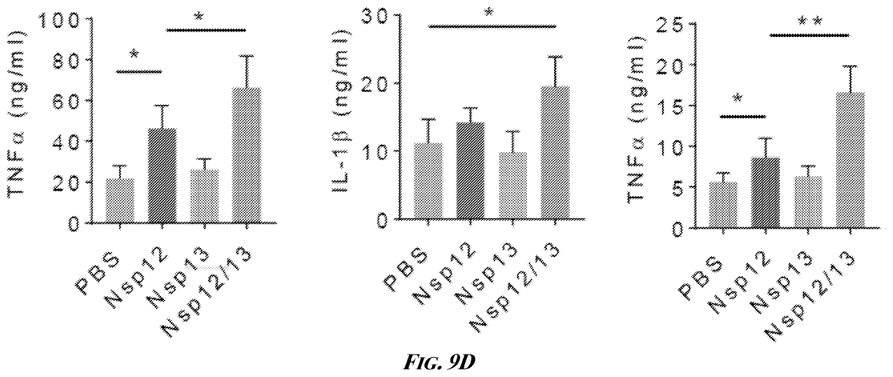

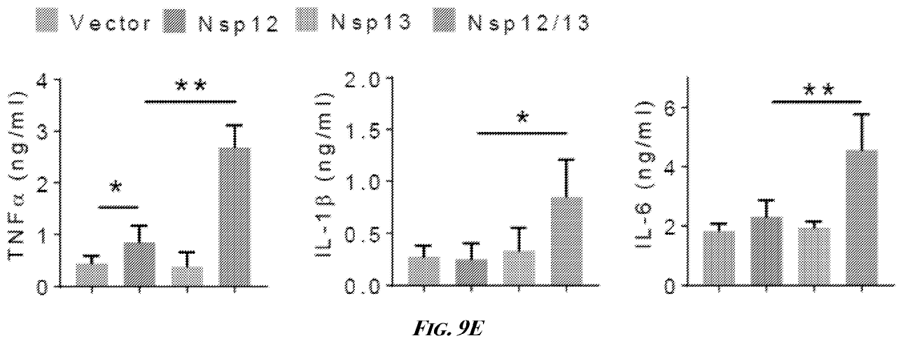

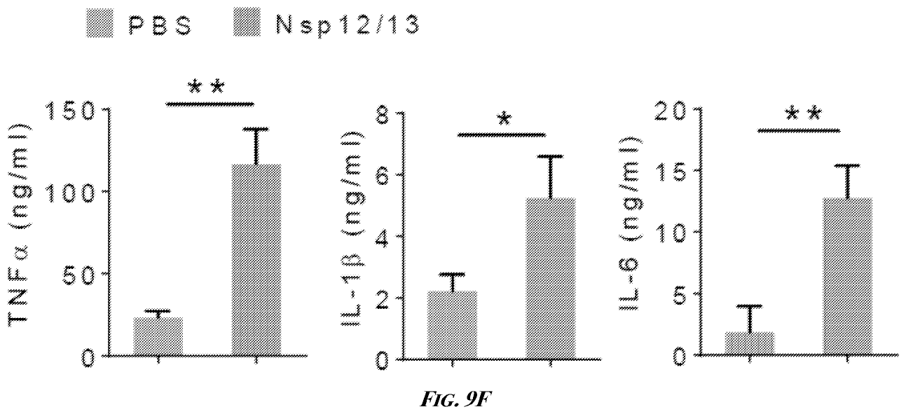

To further investigate the effect of exosomes with viral protein in vivo, exosomes isolated from mouse lung epithelial LCC1 cells transfected with various viral genes were administrated to mice by direct intratracheal injection (FIG. 3F). The imaging fluorescence signals indicated that exosomes labeled with fluorescent DiR dye were present in lungs and serum as soon as 1 hour after intratracheal administration (FIG. 9A). 12 hours after intratracheal injection, the fluorescent signals diminished. No significant fluorescence appeared in the brain, heart, liver, kidney, or intestine of mice (FIG. 9A).

To further determine whether injected exosomes preferentially targeted immune cells, leukocytes from the lungs of mice treated with fluorescent dye PKH260-labelled exosomes were isolated. Fluorescence-activated cell sorting (FACS) analysis (FIGS. 3G and 3H) and immunofluorescence (IF) with confocal microscopy (FIGS. 9B and 9C) demonstrated that exosomes were preferentially taken up by F4/80⁺ cells (FIGS. 3G and 9B) and moderately taken up by Gr-1⁺ cells (FIGS. 3H and 9B), but not by CD11b⁺ cells (FIG. 9C). Among F4/80⁺ macrophages and Gr-1⁺ neutrophils, 43.3%±6.8% (mean±standard deviation (SD)) and 28.2%±3.7% (mean±SD) of cells exhibited exosome/PKH26 positivity, respectively (FIG. 3I).

To determine whether injected exosomes have an effect on cytokine production, murine lung tissues were collected and cytokines were quantified with a standard ELISA after administering LCC1 exosomes three times via the intratracheal route. The results suggested that exosomes containing Nsp12 induced TNFα and IL-6 in the lung; whereas Nsp13 alone did not, but injecting exosomes containing both Nsp12 and Nsp13 led to a dramatic enhancement in the production of TNFα, IL-1β and IL-6 (FIG. 3J). These results were also reproducible when mice were treated with exosomes released from Vero E2 cells transfected with Nsp12 and Nsp13 plasmids. The analysis of cytokines in the lung indicated a synergistic effect of the Nsp12 and Nsp13 on the induction of TNFα, IL-1β, and IL-6 (see FIG. 9D).

Cytokine expression in lung F4/80⁺ cells was further assessed since F4/80⁺ cells are the predominant exosome recipient cells. Consistent with results from human U937 monocytes, FACS analysis suggested that Nsp12 induced TNFα and IL-6 in F4/80⁺ cells and Nsp13 enhanced the effect of Nsp12 on the induction of these cytokines (FIG. 3K). Inoculation of exosomes containing both Nsp12 and Nsp13 caused lung alveolar wall thickening and lung inflammation (FIG. 3L). Lung alveolar wall thickening and lung inflammation did not occur when exosomes containing only Nsp12 or Nsp13 were used. In addition, a synergistic effect of exosomes with Nsp12 and Nsp13 was also evidenced by the fact that increasing TNFα and IL-6 occurred not only in the lung but also in the peripheral blood (FIG. 9E) of mice treated with exosomes^{Nsp12Nsp13}. The induction of inflammatory cytokines in the C57BL/6 mouse lung was also confirmed by exosomes released from lung primary epithelial cell transfected with Nsp12 and Nsp13 plasmids (FIG. 9F).

It was proposed that Nsp12 might interact with Nsp13 to form the complex that regulates expression of cytokines of exosome recipient cells (FIG. 3M). To test the hypothesis, co-immunoprecipitation (co-IP) was performed utilizing the Nsp12 with Flag tag (pGBW-m4046955) and the Nsp13 with Strep tag (pLVX-EF1alpha-SARS-CoV-2-nsp13-2xStrep-IRES-Puro). First, a full length wide-type Nsp12 gene was constructed from the prokaryotic vector transfected into the eukaryotic vector pAcGFP1-C with a CMV promoter. After transfection and expression in A549 cells with plasmids of pAcGFP1-C-Nsp12 and pLVX-Nsp13-Strep, Nsp13 protein was pulled down with Strep-Tactin XT magnetic beads and probed to determine if any Nsp12 was in the Nsp13 pulldown complex. This was accomplished using a dot blot and anti-Flag antibody (FIG. 3M). As expected, the results indicated that Nsp12 interacted with Nsp13 in A549 cells. Together, these data indicated that Nsp12 was not only capable of mediating RNA synthesis and replication of the viral genome, but also was cargo in the exosomes and subsequently induced inflammatory cytokines in the exosome recipient cells. Moreover, Nsp12-mediated induction of inflammatory cytokines was shown to be further enhanced by viral Nsp13.

### Example 5

### GELN miRNAs Inhibit the Expression of SARS-CoV-2 Spike and Nsp12

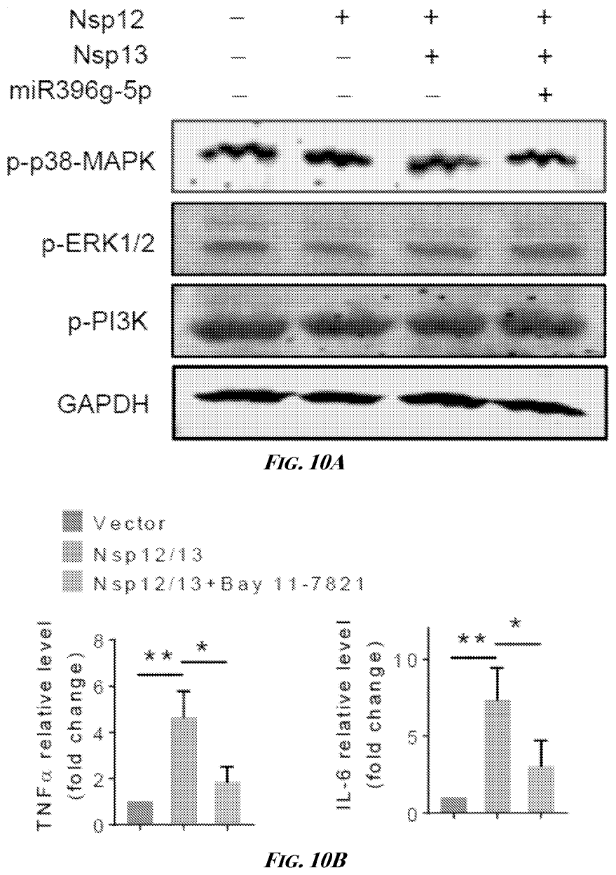

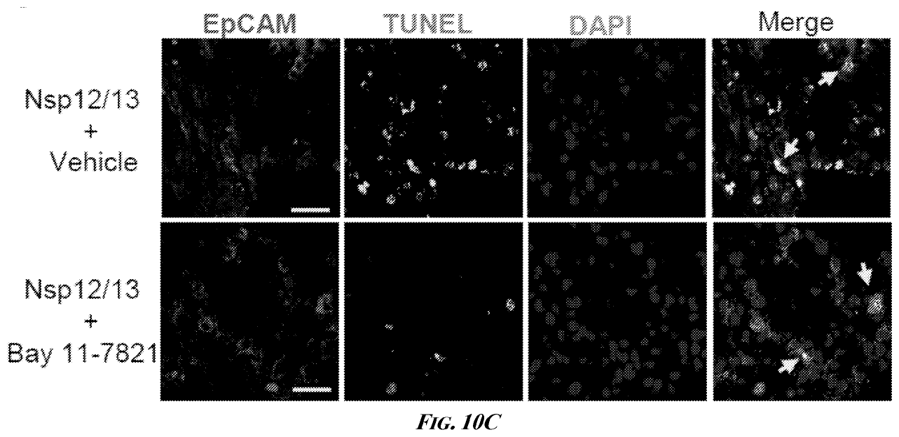

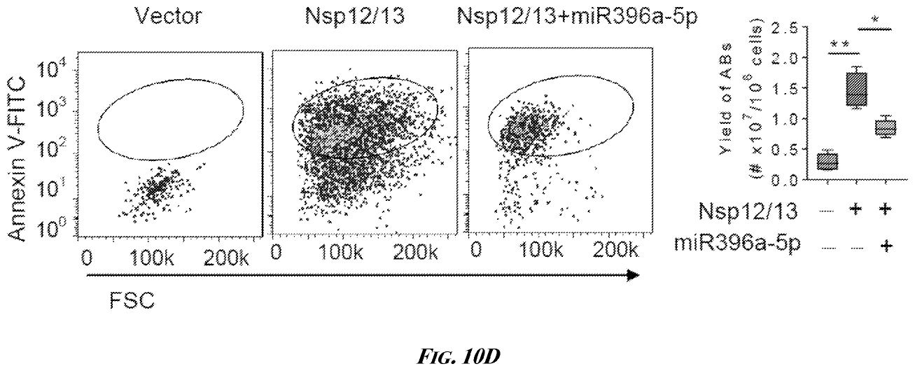

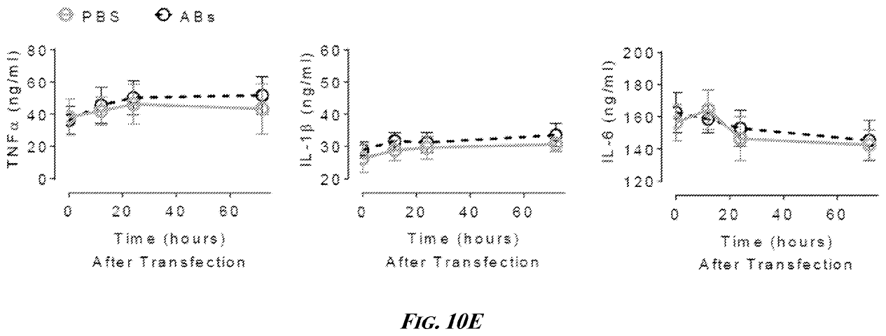

Next, whether a therapeutic strategy could be developed to inhibit the lung cytokine storm and prevent viral Nsp12 induced lung inflammation was tested. GELNs can inhibit mouse colitis via GELN miRNA interaction with gut bacterial mRNA. Therefore, whether GELN miRNA could bind to and inhibit SARS-CoV-2 mRNA expression was tested. ELNs from ginger root were first purified using differential ultracentrifugation and a sucrose gradient technique (FIG. 10A). Next generation sequencing (NGS) analysis of small RNA in the ginger root tissue and in ginger-derived ELNs (GELNs) identified 2.2 million and 3.6 million miRNA read outs of 32 million and 42 million total reads, respectively (FIG. 4A). Combining the presently disclosed sequencing data (which has been deposited in NCBI Gene Expression Omnibus (GEO) database with accession number GSE153126) and previous sequencing data, 2,262 of the miRNAs exceeded the minimum confidence thresholds (cutoff 50 reads) and have been mapped to the entire miRNA database. Of the miRNAs, 532 are higher in GELNs and 1,280 of the miRNAs are higher in ginger tissue (FIG. 4B). Further analysis on the cluster (FIG. 4C), abundance (FIG. 4D), composition (FIG. 4E) of miRNAs revealed significant differences between GELNs and ginger tissue, human, and mouse. The miRNA cargo in GELNs was more enriched than in ginger tissue, in contrast, the ginger tissue had more tRNA compared to the GELNs.

To further explore the therapeutic effects of miRNA against SARS-CoV-2, strict seed sequence lengths of nine nucleotides (nt) in GELN miRNAs were used as criteria to search for the genome sequence of SARS-CoV-2 (NC 045512). 9 nucleotides (nt) were chosen although 6 nt is the minimum requirement and the 6-8 nt long seed sequence of the miRNA is sufficient to bind the target mRNA. 188 GELN miRNAs (black bars in FIG. 4F) that were predicted to bind to genes of SARS-CoV-2 were identified across the SARS-CoV-2 viral genome except for the genes encoding Nsp7, Nsp11, E, Orf8 and Orf10 (FIG. 4F). Considering the world-wide prevalence of SARS-CoV-2 infection and the apparent lack of immunity to prevent SARS-CoV-2 infection, host miRNAs that are sequence homologues to GELN miRNA may not play a critical role in the inhibition of viral gene expression, in particular Nsp12, which is an essential gene for viral replication. Therefore, GELN miRNAs (second row of vertical bars in FIG. 4F) bearing the seed sequences that map to the human or mouse miRNA database were excluded from further analysis. The remaining 135 miRNAs were unique for GELNs were used as miRNAs to target viral genes.

GELN miRNAs as well as their seed sequences that were predicted to bind to the genes of SARS-CoV-2 are listed in Table 2. Interestingly, very few of the human or mouse miRNAs were predicted to bind to SARS-CoV-2 genes, but more miRNAs with matching sequences were found in GELNs, especially for the SARS-CoV-2 Nsp12 and Spike (S) genes. Also, some of the GELN miRNAs may bind to multiple sites of a single viral gene (FIG. 4F).

As a proof-of-concept, the effects of GELN rlcv-miR-rL1-28-3p and aly-miR396a-5p on the appropriate viral gene expression were further tested. An alignment of sequences of nucleotides using the Basic Local Alignment Search Tool (BLAST) indicated that GELN rlcv-miR-rL1-28-3p was predicted to bind to two sites of the S gene (FIG. 4G) and aly-miR396a-5p was predicted to bind to the Nsp12 gene (FIG. 4H). To confirm this prediction, pcDNA3-SARS-CoV-2-S-GFP and CoV-2-Nsp12-2xStrep were co-transfected into A549 cells with GELN rlcv-miR-rL1-28-3p and aly-miR396a-5p, respectively. The expression of S and Nsp12 was significantly down-regulated by rlcv-miR-rL1-28-3p and aly-miR396a-5p as visualized with GFP and StrepTactin-HRP conjugate (FIGS. 4I and 4J).

### Example 6

### GELN aly-miR396a-5p Inhibits NF-κB Mediated Inflammation and apoptosis in the Lung of Mice Intratracheally Injected with Exosomes^{Nsp12Nsp13}

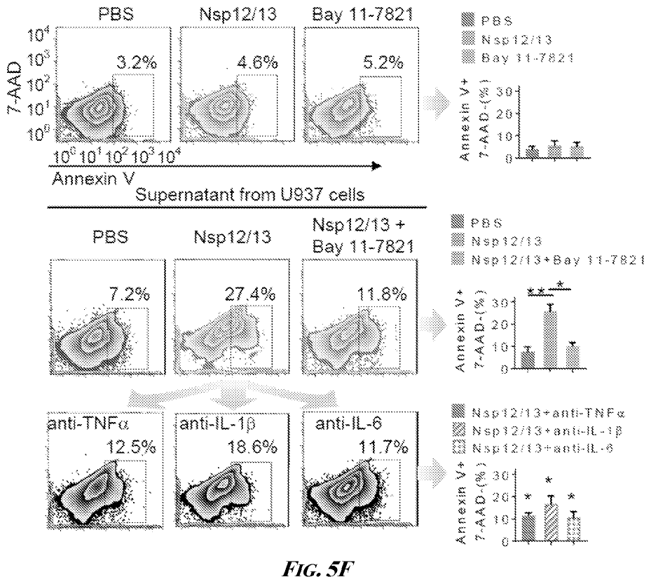

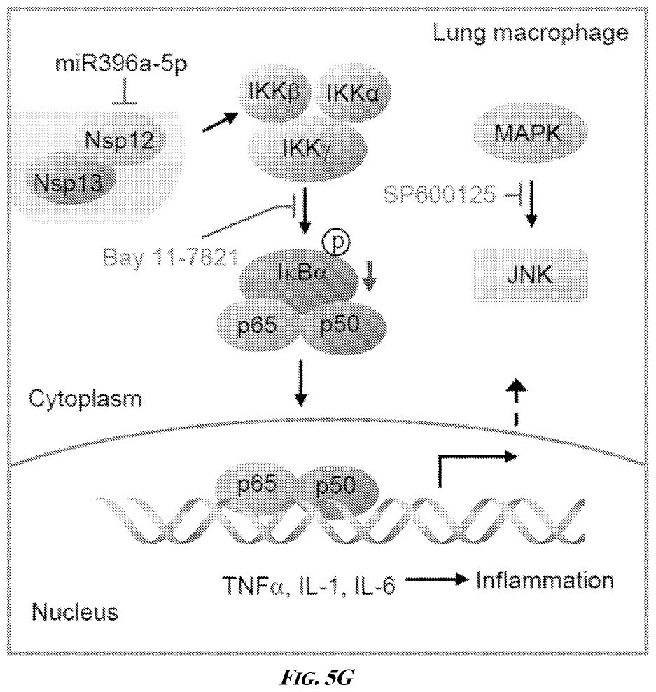

Lung inflammation and apoptosis leading to acute respiratory distress syndrome (ARDS) are a hallmark of COVID19; however, identification of the specific pathways that viral products induce to elicit lung inflammation and apoptosis are still unknown. Based on the literature, activation of mitogen-activated protein kinase (MAPK, p38), extracellular-signal-regulated kinase (ERK) 1/2 (p44/42), c-Jun N-terminal kinase (JNK), phosphatidylinositol 3-kinase (PI3K) as well as nuclear factor (NF)-κB (p65) are all involved in virally induced inflammation and apoptosis. Western blot analysis indicated that mice intratracheally administered exosomes^{Nsp12Nsp13} induced more phosphorylated P65 (a subunit of NF-κB) and JNK in mouse lung macrophages compared to Nsp12 alone (FIG. 5A). There is no evidence suggesting the induction of phosphorylated p38, ERK1/2, or PI3K (FIG. 10A). Activation of NF-κB mainly occurs via IKK-mediated phosphorylation of IκB-α. The presently disclosed results showed that more phosphorylated IκB kinase (IKK)-β and IκB-α were induced by exosomes^{Nsp12Nsp13} (FIG. 5A). Importantly, GELN aly-miR396a-5p treatment prevented the exosomes^{Nsp12Nsp13} mediated activation of NF-κB. Activation of JNK was also detected in the macrophages of mice treated with the exosomes^{Nsp12Nsp13} (FIG. 5A) and aly-miR396a-5p treatment also prevented the exosomes^{Nsp12Nsp13} mediated activation of JNK (FIG. 5A).

To determine whether NF-κB activated by exosomes^{Nsp12Nsp13} was initiated from activation of IKK, mice were treated with the p-IκB-α inhibitor Bay 11-7821 or the p-JNK inhibitor SP600125 as a control. The treatments were done daily for three days at 5 mg/kg/d (body weight) before mice were intratracheally injected with exosomes^{Nsp12Nsp13}. Lung macrophage NF-κB activity induced by exosomes^{Nsp12Nsp13} was inhibited as a result of the Bay 11-7821 treatment, but not treatment with SP600125, suggesting IKK activated by exosomes^{Nsp12Nsp13} was essential in NF-κB activation (FIG. 5B).

Inhibition of NF-κB activation with Bay 11-7821 was also accompanied by attenuation of inflammatory cytokines induced by exosomes^{Nsp12Nsp13} (FIG. 10B). Cytokines have been reported to induce lung apoptosis which is consistent with the presently disclosed observations that exosomes^{Nsp12Nsp13} treatment enhanced the production of the cleaved caspase-3, caspase-7, and PARP in lung (FIG. 5C). Tunnel staining lung sections further showed that apoptotic cells were induced in the lungs of mice treated with exosomes^{Nsp12Nsp13}, and lung apoptosis induced by exosomes^{Nsp12Nsp13} was reduced as a result of treatment with IκB-α inhibitor Bay 11-7821 but not SP600125 (FIG. 5D). Moreover, flow cytometry and immunofluorescence analysis further suggested that higher percentages of apoptotic Annexin V⁺ (FIG. 5E) and TUNEL⁺ cells (FIG. 10C) in lung epithelial cells (EpCAM⁺) were induced as a result of exosomes^{Nsp12Nsp13} treatment and the induction of lung epithelial cell apoptosis was blocked by Bay 11-7821 but not Sp600125 (FIG. 5E). Collectively, the presently disclosed data suggested that GELN aly-miR396a-5p treatment prevented exosomes^{Nsp12Nsp13} mediated NF-κB activation and lung epithelia cell apoptosis.

Given apoptotic cells can generate apoptotic bodies (Abs; Haanen & Vermes, 1995; Arienti et al., 2019), whether ABs also contributed to induction of inflammatory cytokines was further investigated. The effects of ABs released from lung epithelial cells on the immune response of macrophages were analyzed. Lung epithelial LLC1 cells were transfected with viral genes Nsp12 and Nsp13 as well as aly-miR396a-5p. The exosome^{Nsp12/13} and exosome^{Nsp12/13+miR396a-5p} from the medium were administered to mice via intratracheal injection. The lung epithelial cells were isolated and the ABs purified from the cultured medium was quantified with FACS as Annexin-V positive of 1.0-4.0 μm in size (Masvekar et al., 2019). It was determined that exosome^{Nsp12/13} significantly induced the ABs and aly-miR396a-5p reduced the exosome^{Nsp12/13}-mediated apoptotic effect of lung epithelial cells (FIG. 10D).

To identify whether the ABs released from the lung epithelial cells had an effect on the activity of lung macrophages, 1×10⁸ ABs released from 1×10⁶ lung epithelia cells were administered to mice via intratracheal injection. The analysis of cytokines with ELISA indicated that ABs failed to modulate the cytokine levels in the lung (FIG. 10E). Collectively, these data suggested that GELN aly-miR396a-5p treatment prevented exosomes-mediated NF-κB activation and lung epithelia cell apoptosis.

Exosomes^{Nsp12Nsp13} mediated the induction of inflammatory cytokines via activation of NF-κB in macrophages and the lung epithelial cell apoptosis. Whether activated macrophages play a role in the induction of apoptosis of lung epithelial cells was further investigated. FACS analysis indicated that Nsp12/13 and Bay 11-7821 had little influence in lung epithelial A549 cell apoptosis (FIG. 5F, top panel). However, the supernatant from human macrophage U937 cells treated with exosomes^{Nsp12Nsp13} significantly induced the apoptosis of lung epithelial cells (FIG. 5F, middle panel). The induction of apoptosis was inhibited by Bay 11-7821 (FIG. 5F, middle panel), as well as with exosomes^{Nsp12Nsp13} activated macrophage supernatants treated with antibodies against inflammatory cytokines, TNFα, IL-1β and IL-6 (FIG. 5F, bottom panel).

Taken together, these data suggested that lung epithelial cells released SARS-CoV-2 exosomes carrying Nsp12/13 as cargo, leading to activation of macrophage NF-κB and subsequent induction of inflammatory cytokines. The cytokine profile induced by exosomes Nsp12 and 13 not only caused lung inflammation but also induced lung epithelial cell apoptosis (FIG. 5G). GELN aly-miR396a-5p prevented the inflammatory response and cell apoptosis through specifically targeting the inhibition of expression of the Nsp 12 viral gene.

### Example 7

### Intratracheal Delivery of GELN aly-miR396a-5p Inhibits Lung Inflammation Induced by Viral Nsp12

To further determine whether GELN miRNA can inhibit the lung inflammation induced by Nsp12, GELN Aly-miR396a-5p was packed into nanoparticles made from GELN-derived total lipids. Along with rare significant adverse effects (Teng et al., 2018), GELN-derived nanovectors (GNVs) have a number of advantages over nanovectors that are available through commercial markets as demonstrated below,

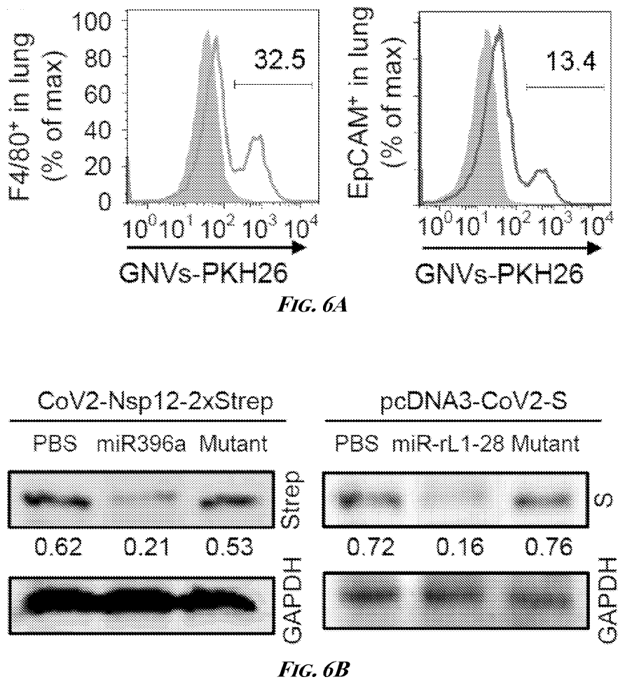

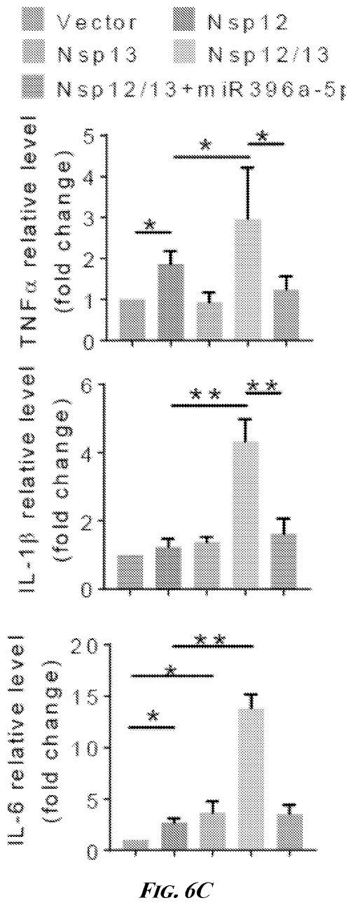

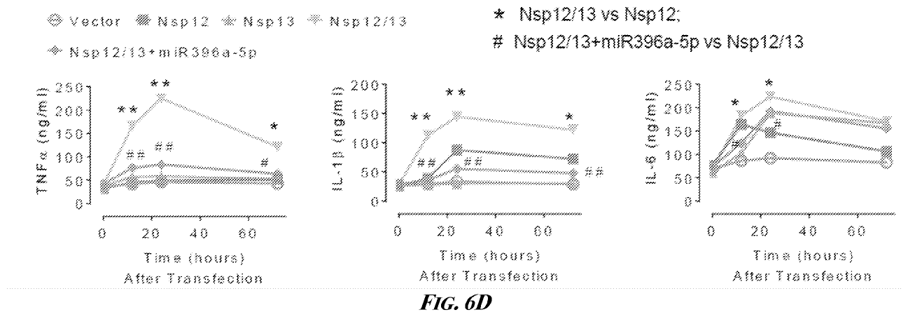

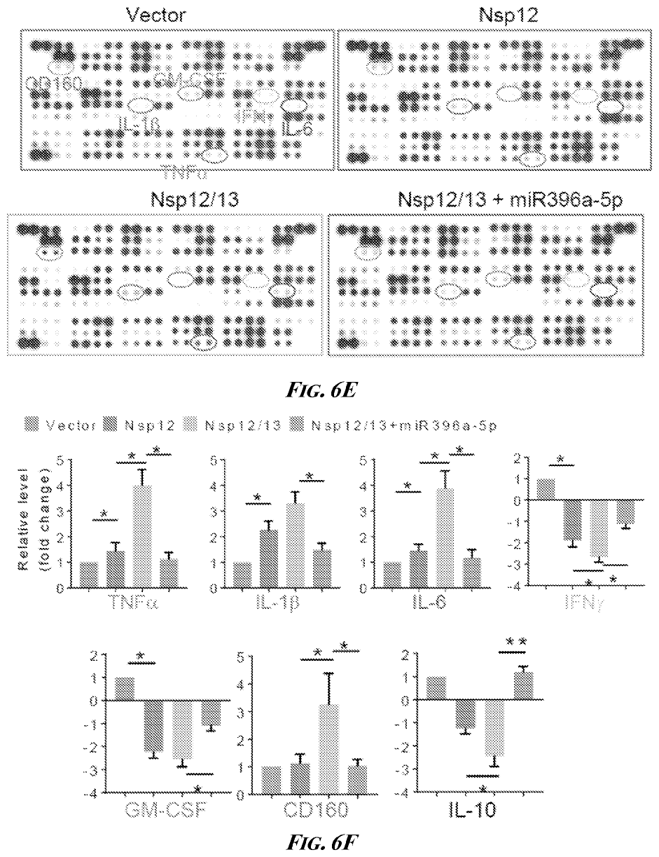

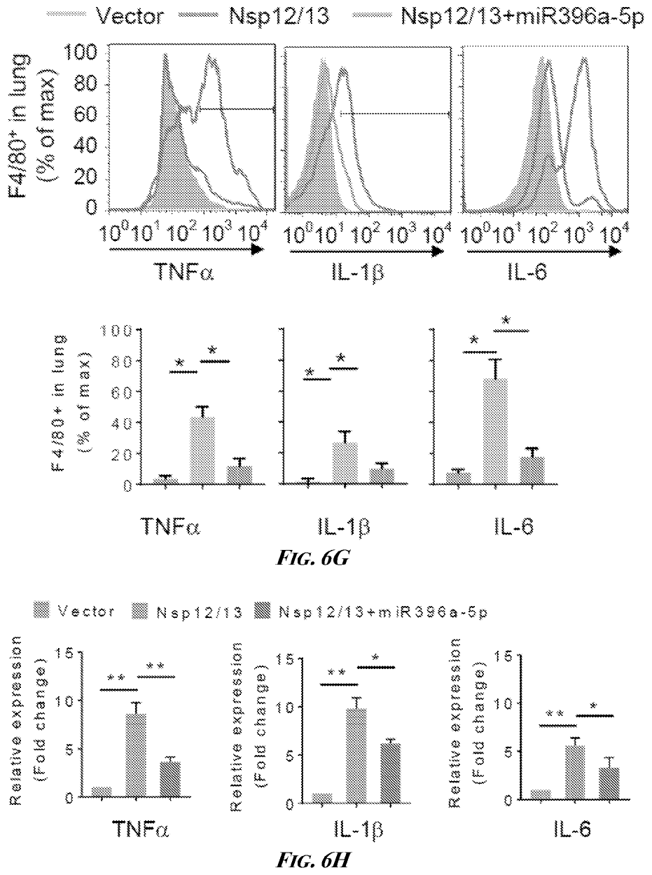

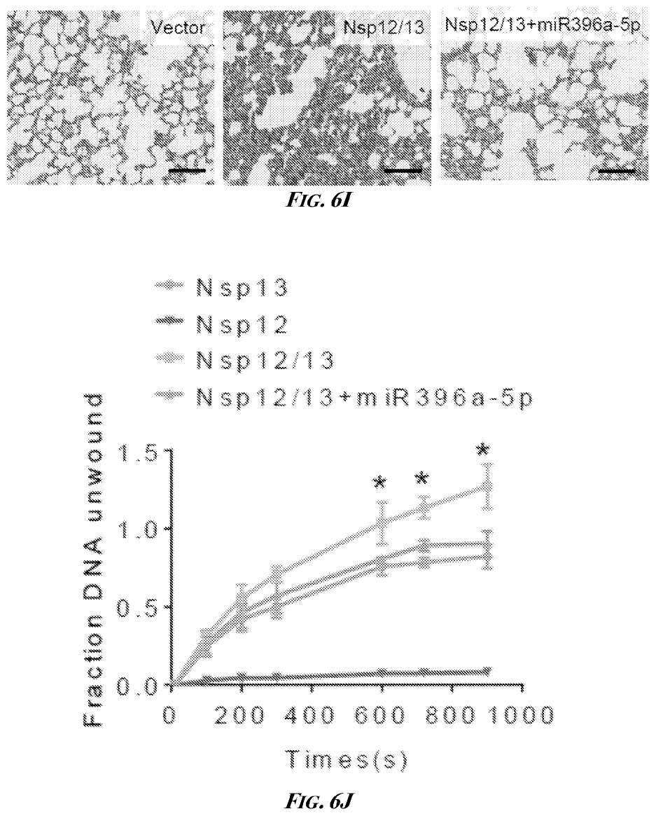

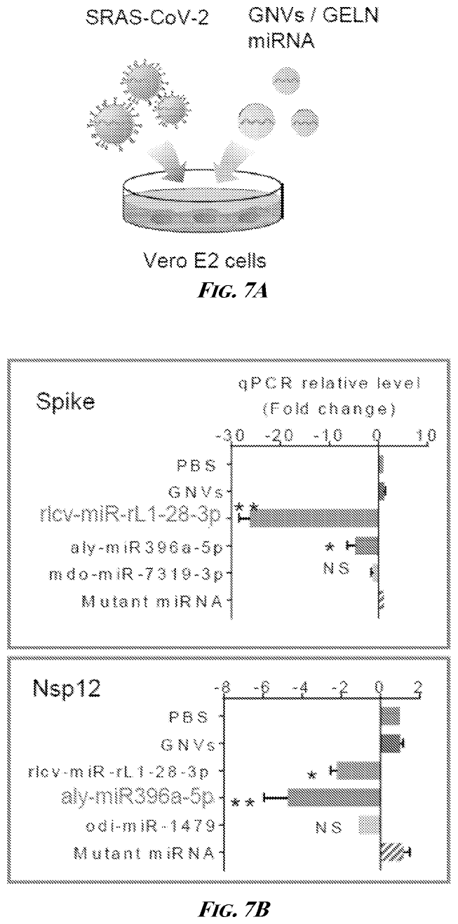

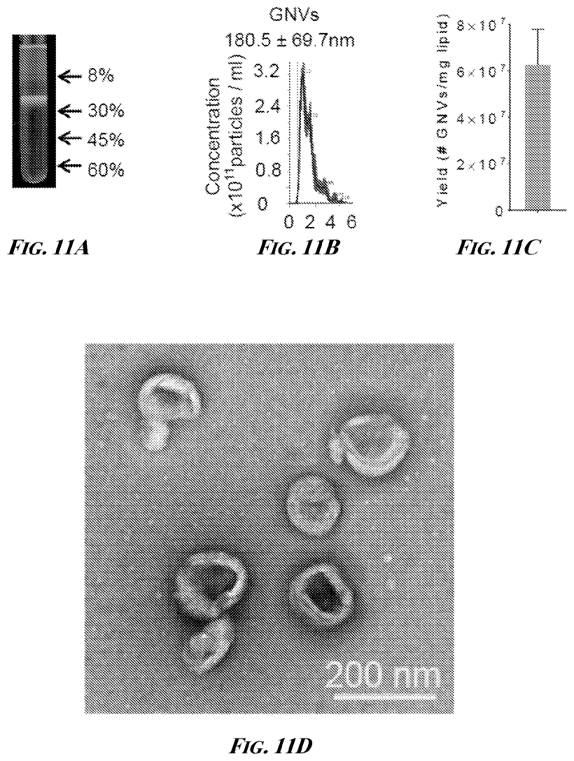

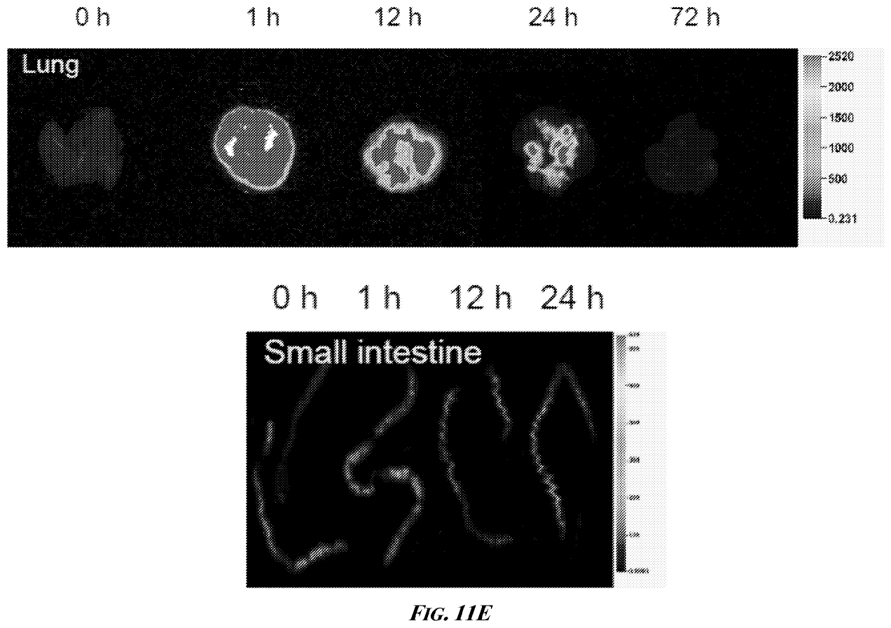

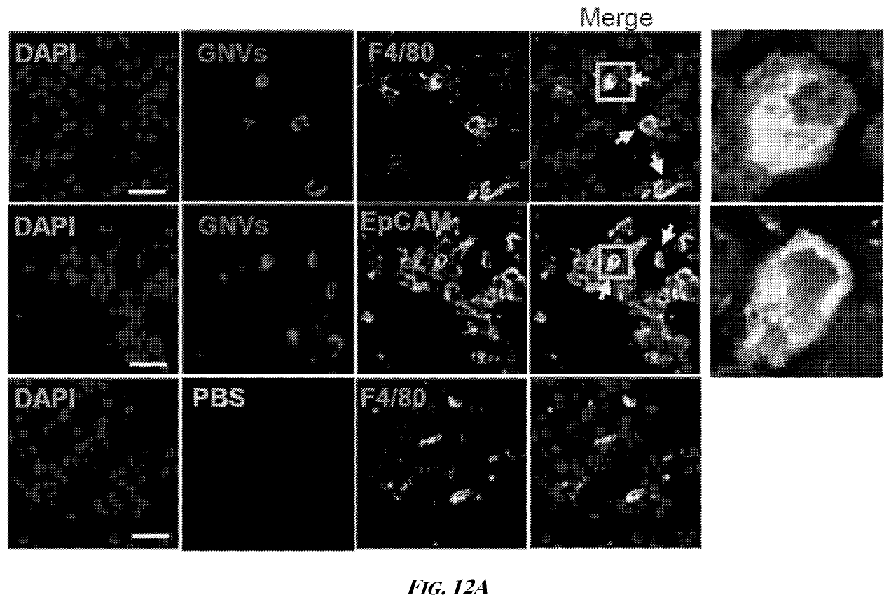

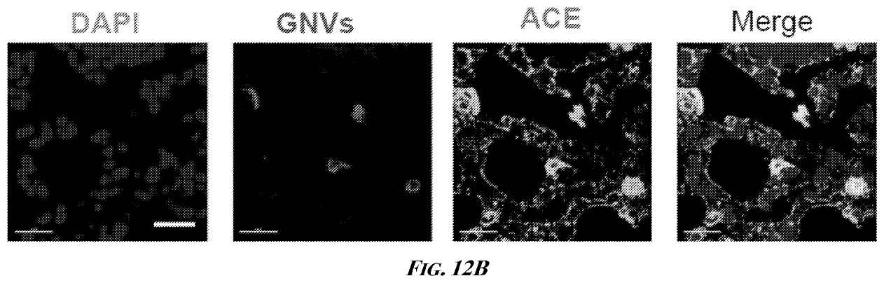

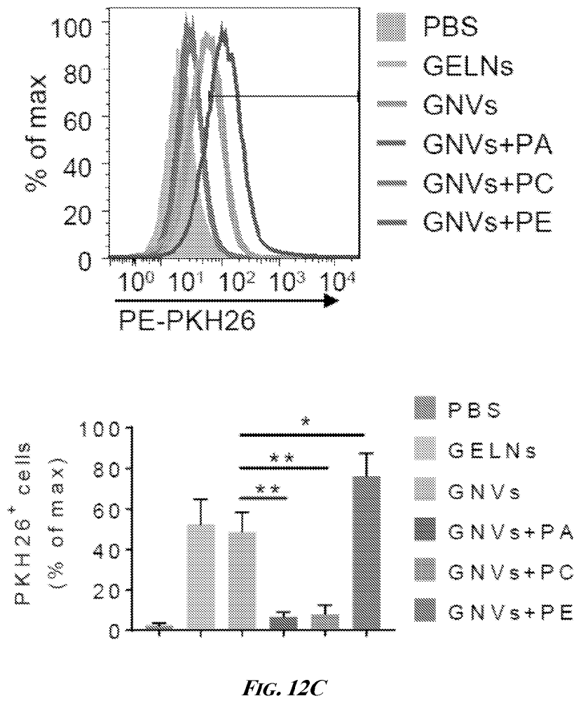

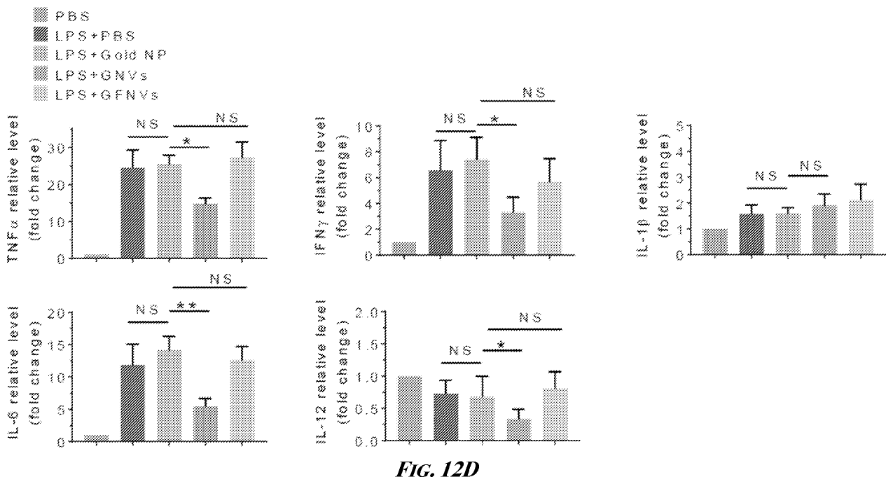

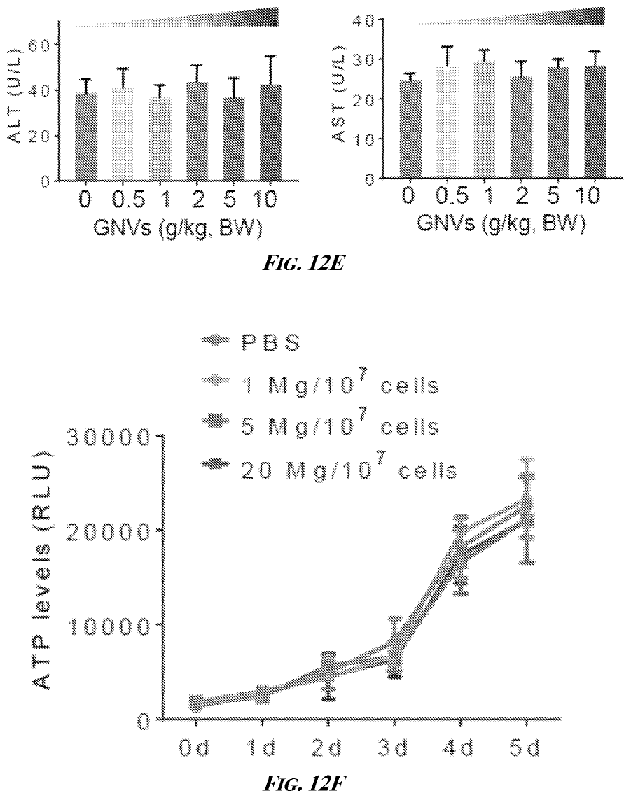

GNVs administrated by intratracheal injection are selectively taken up by lung epithelial cells (host cell for SARS-CoV-2 replication) and macrophages (source for releasing inflammatory cytokines induced by exosomesNspl2Nsp13). First, GNVsGELNs were purified with sucrose gradient centrifugation of ginger juice (FIG. 11A) using a method as described in Teng et al., 2018 and GNVs were generated with total lipids extracted from GELNs using a ultrasonication method as described in Zhuang et al., 2015. The GNVs were further characterized using NTA analysisNanoSight NS300 for size distribution, concentration (FIG. 11B), yield (FIG. 11C) and then electron microscopically examined (FIG. 11D). One hour after intratracheal administration of GNVs in mice, the DiR fluorescent dye labeled GNVs/DiR signal was detectable in the lungs and lasted up to 24 h (FIG. 11E, left panel). Imaging of the small intestine excluded misplacement of the esophagus by the intratracheal injection (FIG. 11E, right panel). Comparing the characteristics of GNVs and GELNs based on size distribution and morphological features from transmission electron microscopy (TEM) analysis, visible differences between GNVs and GELNs were not observed. FACS analysis indicated that PKH26⁺GNVs were taken up by both F4/80⁺ macrophages and EpCAM⁺ lung epithelial cells (FIG. 6A). This result was further confirmed by fluorescence co-localization analysis using confocal microscopy (FIG. 12A). Moreover, the immunofluorescence revealed that the cells in lung with high expression of ACE2 preferentially take up GNVs (FIG. 12B). Given that the lipid of nanoparticles influences target cells uptake (Teng et al., 2018), the effects of three predominate lipids constituting the nanoparticles, i.e., phosphatidic acid (PA), phosphatidylcholine (PC), phosphatidylethanolamine (PE), on the uptake of GNVs/PKH26 was examined using the flow cytometry. It was found that additional PE promoted the GNVs uptake by A549 cells, whereas PA and PC inhibited uptake (FIG. 12C).

Anti-inflammation effects as demonstrated in a lipopolysaccharide (LPS)-induced septic mouse model were also examined. In contrast to gold nanoparticles (NP) that are widely used for chemotherapeutic drug delivery, the GNVs anti-inflammatory effect was demonstrated in an LPS induced lung cytokine storm using the mouse septic model (FIG. 12D) without induced side effects observed. To estimate the GNV-related liver toxicity and adverse effects on cells, the level of alanine aminotransferase (ALT) and aspartate aminotransferase (AST) was measured in the serum and cell proliferation. ENVs exhibited neither toxicity in vivo (FIG. 12E) nor growth influence on the lung epithelial cells (FIG. 12F).

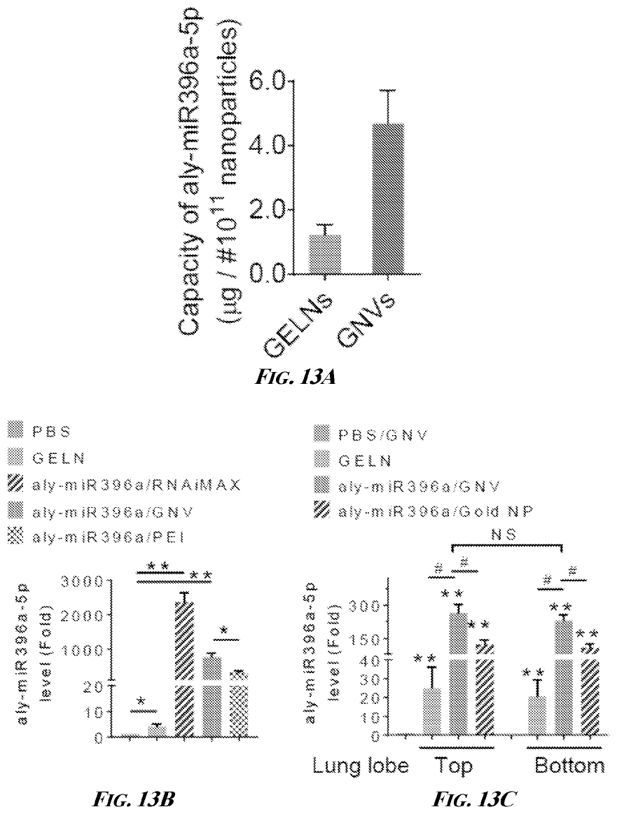

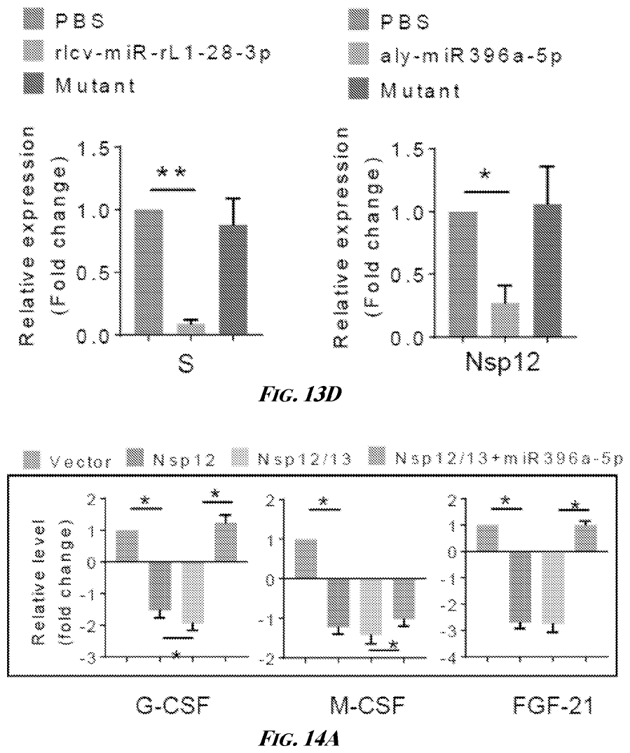

To further test whether GNVs can deliver miRNA to the lung, evaluated the packing efficiency of aly-miR396a-5p in GNVs was evaluated using a quantitative PCR (qPCR) assay. The cargo aly-miR396a-5p in GNVs is 1.22±0.32 μg per 10¹¹ nanoparticles. With 10 μg of aly-miR396a-5p and 200 μmol of GELN-derived total lipids, 4.68±1.03 μg of aly-miR396a-5p per 10¹¹ nanoparticles can be generated (FIG. 13A). Moreover, the transfection efficiency of GNVs carrying aly-miR396a-5p was further evaluated. An in vitro PCR analysis indicates that the miRNA level delivered with GNVs was higher than delivered with poly(ethylenimine) (PEI) which is commonly used to deliver therapeutic mRNAs; the transfection reagent RNAiMax exhibited the highest transfection efficiency (FIG. 13B). In vivo delivery efficiency of aly-miR396a-5p packaged in GNVs was compared with gold NP. After intratracheal injection, the level of aly-miR396a-5p packed in GNVs was higher than the miRNA packed with gold NP (FIG. 13C). A difference in the miRNA level between the top and bottom lobes of the lungs was not observed (FIG. 13C). This result suggested that the GNVs administrated by intratracheal injection were distributed throughout the entire lung. Immuno-blot and qPCR analysis from in vivo experiments suggested that the expression of viral Nsp12 and S was inhibited by GELN aly-miR396a-5p and rlcv-miR-rL1-28-3p packed in GNVs, respectively (FIGS. 6B and 13D).

Whether the aly-miR396a-5p delivered by GNVs inhibited the expression of inflammatory cytokines induced by Nsp12 of SARS-CoV-2 was then investigated. Human monocyte U937 cells were transfected with Nsp12 and/or Nsp13, simultaneously, in the presence or absence of aly-miR396a-5p packed in GNVs,. ELISA results demonstrated that aly-miR396a-5p caused a remarkably negative effect against induction of TNFα, IL-1β, and IL-6 (FIG. 6C). Mice were then exposed to Nsp12, Nsp13, and aly-miR396a-5p through intratracheal administration of exosomes^{Nsp12Nsp13} as well as GELN aly-miR396a-5p. Exosomes^{Nsp12Nsp13}-induced TNFα, IL-1β, and IL-6 in lung peaked 24 hours after inoculation of exosomes; aly-miR396a-5p had an inhibitory effect on the expression of inflammatory cytokines (FIG. 6D).

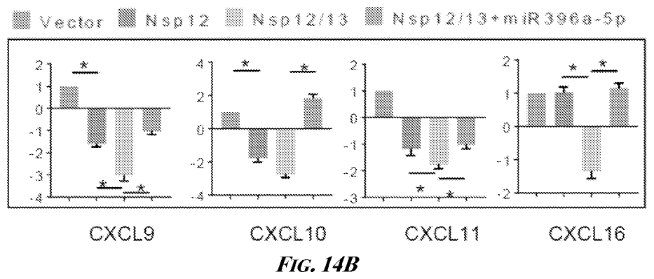

The results generated from cytokine array analysis further demonstrated that besides modulation of TNFα, IL-1β and IL-6, exosomes^{Nsp12Nsp13} significantly lowered the level of interferon γ (IFNγ) and IL-10, and aly-miR396a-5p prevented the reduction of IFNγ (FIGS. 6E and 6F). Moreover, a number of proteins involved in cell growth, including granulocyte macrophage colony-stimulating factor (GM-CSF), G-CSF, M-CSF and fibroblastic growth factor (FGF)-21, were found to be down-regulated by exosomes^{Nsp12Nsp13} (FIGS. 6E, 6F, 14A, and 14B). The levels of proteins in the chemokine (C-X-C motif) ligand (CXCL) family including CXCL9, CXCL10, CXCL11, and CXCL16 were decreased as well due to exosomes^{Nsp12Nsp13} treatment; whereas the expression of CD160 that involved cytolytic effector activity on natural killer (NK) cells was increased (FIGS. 6E and 6F).

The effect of exosomes^{Nsp12Nsp13}on cytokine production and the reversal of the effect by aly-miR396a-5p in F4/80⁺ cells was confirmed by FACS analysis (FIG. 6G) as well as in transcription level qPCR analysis (FIG. 6H). Histological examination demonstrated that the pulmonary inflammation caused by exosomes^{Nsp12Nsp13} was improved by aly-miR396a-5p (FIG. 6I).

It was previously reported that Nsp12 can enhance the unwinding activity of Nsp13 in the replication and transcription of SARS-CoV (Canton et al., 2018). Although the synergy of Nsp12 and Nsp13 on the inflammatory response was revealed here, to further determine whether GELN aly-miR396a-5p modulates the synergy of Nsp12 and Nsp13 via helicase activity, a DNA helicase assay was used. The Cy3-modified DNA strand exposed to helicase can be separated from the dsDNA that is modified using a black-hole quencher (BHQ; see e.g., U.S. Pat. No. 10,824,087; incorporated herein by reference in its entirety). Complete separation of the Cy3-modified reporter strand bound to single-stranded DNA (ssDNA) without BHQ and was thus detected as an increase in total fluorescence. The results suggested that Nsp12 enhanced the unwinding activity of Nsp13 and aly-miR396a-5p abolished the unwinding activity mediated by Nsp12 (FIG. 6J).

### Example 8

### GELN miRNAs Inhibit SARS-CoV-2 Cytopathogenic Effect (CPE) in Vero E6 Cells by Inhibiting the Expression of the Viral S and Nsp12

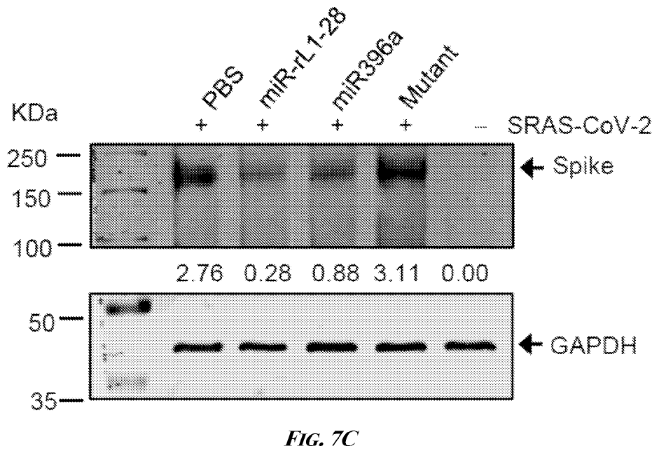

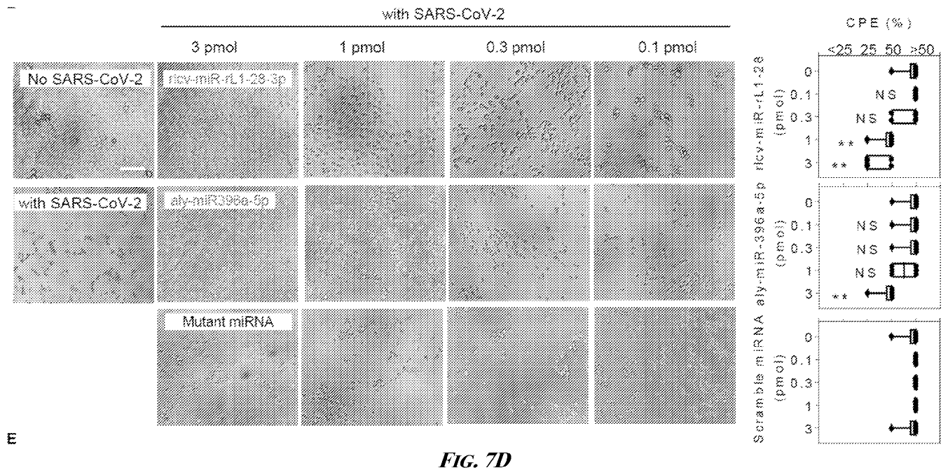

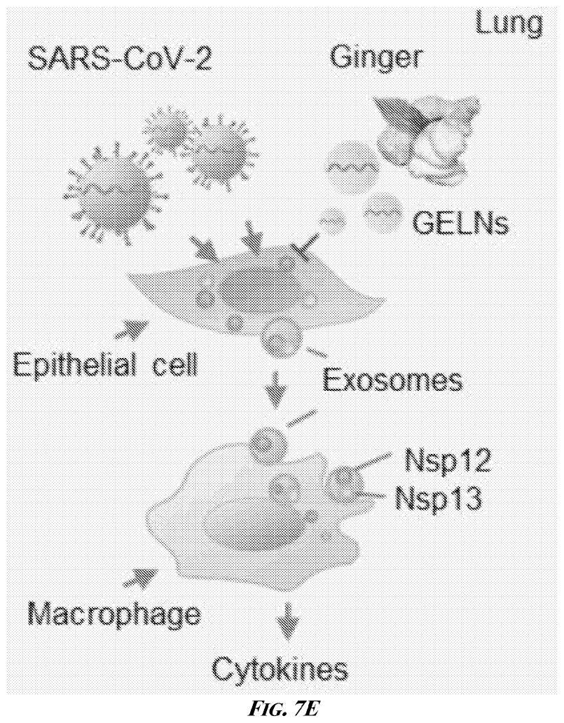

To further investigate whether GELN miRNAs that exhibited potent viral gene inhibitory activity could significantly inhibit viral replication, Vero E6 cells (2×10⁴ cells per well) were exposed to SARS-CoV-2 at a concentration of 60 plaque forming units (pfu) per well for a multiplicity of infection (MOI) of 0.003. GELN rlcv-miR-rL1-28-3p and aly-miR396a-5p, which inhibit the expression of S gene and Nsp12 genes, respectively, were packed into GNVs and added to virus infected Vero E6 cells (FIG. 7A). On day 3 post-infection, the expression of spike protein and Nsp12 in SARS-CoV-2 infected Vero E6 cells with or without GELN miRNAs was estimated by qPCR. The result indicated that both viral S and Nsp12 expression were reduced by either rlcv-miR-rL1-28-3p or aly-miR396a-5p (FIG. 7B). However, mdo-miR-7319-3p and odi-miR-1479 did not affect the expression of spike protein and Nsp12 despite having seed sequences that could potentially bind to sites to spike and NSp12, respectively. Western blot further confirmed that both rlcv-miR-rL1-28-3p and aly-miR396a-5p reduced the expression of spike protein in transfected Vero E2 cells (FIG. 7C).

To assess virus-induced cytopathogenic effect (CPE), Vero cells were seeded in 96-well plates at a multiplicity of infection (MOI) of 0.003. In contrast to normal cells, which had no CPE (FIG. 7D), cells infected with SARS-CoV-2 exhibited evident morphological changes as shown by the rounded cell bodies and their elongated shape (FIG. 7D, left panel). These CPE-positive cells detached from the plate and were reduced by rlcv-miR-rL1-28-3p and aly-miR396a-5p in a dose dependence manner, but the mutant miRNA had no evident influence on SARS-CoV-2-induced CPE (FIG. 7D, right panel).

Taken together with the evidence that non-human intragenic GELN miRNA can inhibit SARS-CoV-2 replication through specific binding and limiting of viral gene expression, including that of viral spike protein and RNA polymerase Nsp12 (FIG. 7E), the presently disclosed subject matter thus provides an innovative and safe strategy for use with the coronavirus infection.

### Discussion of the Examples

Inflammation is a hallmark of septic shock and infection with various bacteria and viruses including but not limited to the severe acute respiratory syndrome coronavirus 2 virus SARS-CoV-2. Regarding SARS-CoV-2, the most vulnerable populations during the current pandemic are elderly individuals, whose immune systems naturally start to decline with age, and immunosuppressed individuals. Their weaker immune responses allow the virus to take up residence in the lungs. As a result, their immune systems start to overrespond by recruiting more inflammatory cells to the lungs, which release inflammatory cytokines in a frantic but frequently futile attempt to wipe out the virus. This drives a continuous release of more and more inflammatory cytokines (referred to as the “cytokine storm”) that can cause substantial cell death in the lungs, resulting in the most severe infections, acute respiratory distress syndrome, and even death.

Therefore, a delivery system that allows for targeted delivery of therapeutic agents that can block virus replication that induces lung inflammation would be advantageous. The major obstacles for targeting therapy are efficient delivery of the therapeutic agents to the lung, where the inflammation takes place and where SARS-CoV-2 replication occurs. As described herein, intranasal administration of G-Exo and T-Exo exosomes can inhibit lung inflammation and can prevent septic shock-induced mortality. These results can be directly applied to treating lung inflammation-related diseases such as but not limited to COVID-19 via a non-invasive and convenient methodology.

As disclosed herein, a new biological activity of viral Nsp12 by which macrophages are activated through the NK-κB mediated pathway has been identified. Nsp12 is delivered by lung epithelial cell exosomes to macrophages, leading to the activation of the macrophages via NF-κB. Activated macrophages then release a number of inflammatory cytokines that contribute to lung inflammation. It was also found that exosomes carrying Nsp13 can synergize with Nsp12 in terms of activation of NF-κB. The metabolites released from exosomes^{Nsp12Nsp13}-activated macrophages cause lung epithelial cell apoptosis.

It was discovered that a large number of ginger exosomal miRNAs were predicted to bind to multiple sites of the SARS-CoV-2 viral genome and that these ginger miRNAs have no homologous sequences shared with host miRs. This finding is significant in that no ginger exosome homologue miRNAs shared sequences with viral host cell-derived miRNAs, which in turn means that it is unlikely that side effects would occur.

The results presented herein also demonstrated that GNVs could be taken up by lung macrophages and lung epithelial cells. Targeted delivery of ginger miRNA to lung epithelial cells inhibited the expression of Nsp12 and subsequently prevents exosomes^{Nsp+} mediated lung inflammation. Targeting to macrophages could lead to inhibiting the activation of macrophages and subsequently alter the composition of the metabolites of exosomes^{Nsp12Nsp13} activated macrophages. Altering the composition of the metabolites leads to a decrease of apoptosis of lung epithelial cells. Interestingly, the presently disclosed data suggested that TNFα, IL-6, and IL-1β in the context of supernatants from exosomes^{Nsp12Nsp13} activated macrophages plays a role in promoting lung epithelial cell apoptosis.

In conclusion, the presently disclosed subject matter provides a basis for lung targetable edible exosome-like vectors to treat lung inflammatory-related diseases, including but not limited to the use of nanoparticle-based therapies (e.g., ginger-derived nanoparticles) that can be employed to inhibit activation of NF-κB mediated pathways that play a crucial role in many inflammation-related diseases, including COVID-19.

### REFERENCES

All references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
Aftab et al. (2020) Analysis of SARS-CoV-2 RNA-dependent RNA polymerase as a potential therapeutic drug target using a computational approach. J Transl Med 18:275.
Arienti et al. (2019) Regulation of Apoptotic Cell Clearance During Resolution of Inflammation. Front Pharmacol 10:891.
Benjamini & Hochberg (1995) Controlling the false discovery rate: a practical and powerful approach to multiple testing. Journal of the Royal Statistical Society. Series B (Methodological) 57:289-300.
Berezikov et al. (2006) Diversity of microRNAs in human and chimpanzee brain. Nat Genet 38:1375-1377.
Berkow et al. (1997) The Merck Manual of Medical Information, Home ed. Merck Research Laboratories, Whitehouse Station, New Jersey, United States of America.
Blanco-Melo et al. (2020) Imbalanced Host Response to SARS-CoV-2 Drives Development of COVID-19. Cell 181:1036-1045.
Cantoni & Rossman (2018) Ebolaviruses: New roles for old proteins. PLoS Neglected Tropical Diseases 12:e0006349.
Chettimada et al. (2018) Exosome markers associated with immune activation and oxidative stress in HIV patients on antiretroviral therapy. Scientific Reports 8:7227.
Cinatl et al. (2003) Glycyrrhizin, an active component of liquorice roots, and replication of SARS-associated coronavirus. Lancet 361:2045-2046.
Coenen-Stass et al. (2018) Evaluation of methodologies for microRNA biomarker detection by next generation sequencing. RNA Biol 15:1133-1145.
Consortium et al. (2017) EV-TRACK: transparent reporting and centralizing knowledge in extracellular vesicle research. Nat Methods 14:228-232.
Dowdy & Wearden (1983) Statistics for Research, John Wiley & Sons, New York, N.Y., United States of America.
Duch et al. (1998) Volatile anesthetics significantly suppress central and peripheral mammalian sodium channels. Toxicol Lett 100-101:255-263.
Ebadi (1998) CRC Desk Reference of Clinical Pharmacology. CRC Press, Boca Raton, Fla., United States of America.
Ellwanger et al. (2011) The sufficient minimal set of miRNA seed types. Bioinformatics 27:1346-1350.
Freireich et al. (1966) Quantitative comparison of toxicity of anticancer agents in mouse, rat, hamster, dog, monkey, and man. Cancer Chemother Rep. 50:219-244.
Godoy et al. (2018) Large Differences in Small RNA Composition Between Human Biofluids. Cell Rep 25:1346-1358.
Goodman et al. (1996) Goodman & Gilman's The Pharmacological Basis of Therapeutics, 9th ed. McGraw-Hill Health Professions Division, New York, N.Y., United States of America.
Haanen & Vermes (1995) Apoptosis and inflammation. Mediators Inflamm 4:5-15.
Harcourt et al. (2020) Severe Acute Respiratory Syndrome Coronavirus 2 from Patient with Coronavirus Disease, United States. Emerg Infect Dis 26:1266-1273.
Herold et al. (2008) Lung epithelial apoptosis in influenza virus pneumonia: the role of macrophage-expressed TNF-related apoptosis-inducing ligand. J Exp Med 205:3065-3077.
Higuchi et al. (2018) Role of exosomes as a proinflammatory mediator in the development of EBV-associated lymphoma. Blood 131:2552-2567.
Hoffmann et al. (2020) SARS-CoV-2 Cell Entry Depends on ACE2 and TMPRSS2 and Is Blocked by a Clinically Proven Protease Inhibitor. Cell 181:271-280.
Huang et al. (2020) Exosomes facilitate transmission of Enterovirus A71 from human intestinal epithelial cells. The Journal of Infectious Diseases 222:456-469.
Israel (2010) The IKK complex, a central regulator of NF-kappaB activation. Cold Spring Harb Perspect Biol 2:a000158.
Katzung (2001) Basic & Clinical Pharmacology, 8th ed. Lange Medical Books/McGraw-Hill Medical Pub. Division, New York, New York, United States of America.
Kim et al. (2016) In vitro and in vivo neuroprotective effects of cJun N-terminal kinase inhibitors on retinal ganglion cells. Mol Neurodegener 11:30.
Kozomara et al. (2019) miRBase: from microRNA sequences to function. Nucleic Acids Res 47:D155-D162.
Lee et al. (2018) Extracellular Vesicle: An Emerging Mediator of Intercellular Crosstalk in Lung Inflammation and Injury. Front Immunol 9:924.
Leinonen et al. (2010) The sequence read archive. Nucleic Acids Research 39:D19-D21.
Masvekar et al. (2019) Quantifications of CSF Apoptotic Bodies Do Not Provide Clinical Value in Multiple Sclerosis. Front Neurol 10, 1241, doi:10.3389/fneur.2019.01241 (2019).
Miura (2019) Respiratory epithelial cells as master communicators during viral infections. Curr Clin Microbiol Rep 6:10-17.
Miyamoto et al. (2010) Inhibitor of IkappaB kinase activity, BAY 11-7082, interferes with interferon regulatory factor 7 nuclear translocation and type I interferon production by plasmacytoid dendritic cells. Arthritis Res Ther 12:R87.
Mu et al. (2014) Interspecies communication between plant and mouse gut host cells through edible plant derived exosome-like nanoparticles. Molecular Nutrition & Food Research 58:1561-1573.
Mukhamedova et al. (2019) Exosomes containing HIV protein Nef reorganize lipid rafts potentiating inflammatory response in bystander cells. PLoS Pathogens 15:e1007907.
Murphy et al. (2008) Suppression of immediate-early viral gene expression by herpesvirus-coded microRNAs: implications for latency. Proceedings of the National Academy of Sciences USA 105:5453-5458.
Nahand et al. (2020) Exosomal miRNAs: novel players in viral infection. Epigenomics 12:353-370.
Pastuzyn et al. (2018) The Neuronal Gene Arc Encodes a Repurposed Retrotransposon Gag Protein that Mediates Intercellular RNA Transfer. Cell 172:275-288.
PCT International Patent Application Publication Nos. WO 93/25521, WO 98/47491, WO 2013/070324, WO 2019/104242.
Raab-Traub & Dittmer (2017) Viral effects on the content and function of extracellular vesicles. Nature Reviews Microbiology 15:559-572.
Remington et al. (1975) Remington's Pharmaceutical Sciences, 15th ed. Mack Pub. Co., Easton, Pa., United States of America.
Robinson & Oshlack (2010) A scaling normalization method for differential expression analysis of RNA-seq data. Genome Biology 11:1-9.
Sampey et al. (2016) Exosomes from HIV-1-infected Cells Stimulate Production of Pro-inflammatory Cytokines through Trans-activating Response (TAR) RNA. The Journal of Biological Chemistry 291:1251-1266.
Santiana et al. (2018) Vesicle-Cloaked Virus Clusters Are Optimal Units for Inter-organismal Viral Transmission. Cell Host & Microbe 24:208-220.
Speight et al. (1997) Avery's Drug Treatment: A Guide to the Properties, Choice, Therapeutic Use and Economic Value of Drugs in Disease Management, 4th ed. Adis International, Philadelphia, Pa., United States of America.
Sundaram et al. (2019) Plant-Derived Exosomal Nanoparticles Inhibit Pathogenicity of Porphyromonas gingivalis. iScience 21:308-327.
Teng et al. (2016) Grapefruit-derived nanovectors deliver miR-18a for treatment of liver metastasis of colon cancer by induction of M1 macrophages. Oncotarget 7:25683-25697.
Teng et al. (2017) MVP-mediated exosomal sorting of miR-193a promotes colon cancer progression. Nat Commun 8:14448.
Teng et al. (2018) Plant-Derived Exosomal MicroRNAs Shape the Gut Microbiota. Cell Host Microbe 24:637-652.
U.S. Patent Application Publication Nos. 2012/0315324, 2014/0308212, 2017/0035700, and 2018/0362724.
U.S. Pat. Nos. 4,839,177; 4,891,223; 5,326,902; 5,397,574; 5,399,358; 5,399,359; 5,399,362; 5,419,917; 5,422,123; 5,456,921; 5,458,005; 5,458,887; 5,458,888; 5,464,633; 5,472,708; 5,480,792; 5,512,297; 5,525,524; 5,603,956; 5,631,171; 5,679,526; 5,725,883; 5,773,025; 5,824,638; 5,824,799; 5,834,023; 5,837,379; 5,851,776; 5,885,527; 5,885,616; 5,897,876; 5,912,013; 5,916,595; 5,922,615; 5,939,272; 5,947,124; 5,952,004; 5,955,377; 5,985,579; 6,004,582; 6,019,944; 6,077,541; 6,096,340; 6,099,859; 6,099,862; 6,103,263; 6,106,862; 6,110,498; 6,113,855; 6,143,576; 6,180,082; 6,890,763; 6,925,389; 6,989,100; 10,799,457; 10,824,087.
Velandia-Romero et al. (2020) Extracellular vesicles of U937 macrophage cell line infected with DENV-2 induce activation in endothelial cells EA.hy926. PloS One 15:e0227030.
Wang et al. (2013) Delivery of therapeutic agents by nanoparticles made of grapefruit-derived lipids. Nat Commun 4:1867.
Warnes et al. (2009) gplots: Various R programming tools for plotting data. R Package version 2:1.
Xiao et al. (2018) Identification of exosome-like nanoparticle-derived microRNAs from 11 edible fruits and vegetables. PeerJ 6:e5186.
Zhang et al. (2008) Inhibition of the tumor necrosis factor-alpha pathway is radioprotective for the lung. Clin Cancer Res 14:1868-1876.
Zhuang et al. (2015) Ginger-derived nanoparticles protect against alcohol-induced liver damage. J Extracell Vesicles 4:28713.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A method for inhibiting an immune response against a microbial antigen, optionally an immune response against a viral antigen, in a subject in need thereof, the method comprising administering to the subject a composition comprising a plurality of plant-derived exosome-like particles, wherein the plurality of plant-derived exosome-like particles is present in the composition in amounts sufficient to inhibit the immune response against the microbial, optionally virus, antigen in the subject.

2. A method for inhibiting development of septic shock in a subject in need thereof, the method comprising administering to the subject a composition comprising a plurality of plant-derived exosome-like particles, wherein the plurality of plant-derived exosome-like particles are present in the composition in an amount and the administering is via a route sufficient to inhibit development of septic shock in the subject.

3. A method for inhibiting development of cytokine storm in a subject in need thereof, the method comprising administering to the subject a composition comprising a plurality of plant-derived exosome-like particles, wherein the plurality of plant-derived exosome-like particles are present in the composition in an amount and the administering is via a route sufficient to inhibit development of cytokine storm in the subject.

4. The method of any one of claims 1-3, wherein the plurality of plant-derived exosome-like particles are garlic-derived exosomes (G-Exo), turmeric-derived exosomes (T-exo), lemon-derived exosomes (L-Exo), or any combination thereof.

5. The method of any one of claims 1-4, wherein the composition is administered to the subject by inhalation and/or insufflation, optionally where in inhalation and/or insufflation occurs through the nasal cavity.

6. The method of any one of claims 1-5, wherein the microbial antigen is a virus antigen, optionally a coronavirus antigen, further optionally an antigen derived from a SARS-CoV-2 virus.

7. The method of any one of claims 1-6, wherein the subject is a human.

8. The method of any one of claims 1-7, further comprising administering to the subject one or more antimicrobial and/or antiviral treatments and/or one or more immunosuppressive treatments.

9. A composition comprising:
an exosome-derived nanoparticle comprising a first lipid bilayer; and
a second lipid bilayer coating the exosome-like nanoparticle and/or fused with the first lipid bilayer,
wherein the second lipid bilayer comprises a targeting molecule that targets the composition to a cell, tissue, or organ of interest.

10. The composition of claim 9, wherein the exosome-derived nanoparticle encapsulates a therapeutic agent.

11. The composition of claim 9 or claim 10, wherein the second lipid bilayer is derived from a virus, optionally a coronavirus, further optionally a SARS-CoV-2 virus.

12. The composition of any one of claims 9-11, wherein the exosome-derived nanoparticle is derived from an edible plant, optionally a fruit, vegetable, or other plant.

13. The composition of any one of claims 9-12, wherein the exosome-derived nanoparticle is derived from a grape, a grapefruit, a tomato, broccoli, ginger, garlic (G-Exo), turmeric (T-Exo), lemon (L-exo), or any combination thereof.

14. The composition of claim 9, wherein the therapeutic agent is selected from a phytochemical agent, a chemotherapeutic agent, and an antimicrobial agent, optionally an antiviral agent.

15. The composition of claim 14, wherein the therapeutic agent is a phytochemical agent, optionally a phytochemical agent selected from curcumin, resveratrol, baicalein, equol, fisetin, and quercetin.

16. The composition of claim 14, wherein the therapeutic agent is a chemotherapeutic agent, optionally a chemotherapeutic agent selected from the group consisting of retinoic acid, 5-fluorouracil, vincristine, actinomycin D, adriamycin, cisplatin, docetaxel, doxorubicin, and taxol.

17. The composition of claim 14, wherein the therapeutic agent comprises a nucleic acid molecule selected from an siRNA, a microRNA, and a mammalian expression vector.

18. The composition of claim 14, wherein the antiviral agent is a nucleotide or nucleoside analogue, and/or is an anti-retroviral agent.

19. A pharmaceutical composition, comprising the composition of any one of claims 9-18 and a pharmaceutically-acceptable vehicle, carrier, or excipient.

20. A method for treating a viral infection, optionally a coronavirus infection, further optionally a SARS-CoV-2 infection, the method comprising administering to a subject in need thereof an effective amount of a composition of any one of claims 9-18 and/or a pharmaceutical composition of claim 19.

21. A method for targeting a therapeutic to a cell, tissue, or organ of interest, the method comprising administering to a subject in need thereof an effective amount of a composition of any one of claims 9-18 and/or a pharmaceutical composition of claim 19, wherein the composition and/or the pharmaceutical composition comprises a targeting molecule that targets a therapeutic molecule to the cell, tissue, or organ of interest.

22. A method for inhibiting a SARS-CoV-2 induced cytopathogenic effect (CPE), the method comprising administering to a subject in need thereof an effective amount of a composition of any one of claims 9-18 and/or a pharmaceutical composition of claim 19, wherein the composition and/or the pharmaceutical composition comprises an miR396a species, an miR-rL1-28 species, or any combination thereof.

23. The method of claim 22, wherein the composition or the pharmaceutical composition comprises a ginger exosome-like nanoparticle (GELN), a garlic exosome-like particle (G-Exo), a turmeric exosome-like particle (T-Exo), a lemon exosome-like particle (L-Exo), or any combination thereof comprising an miRNA aly-miR396a-5p.
